(19) United States
(12) Patent Application Publication
(10) Pub. No.: US 2024/0408275 A1
(43) Pub. Date: Dec. 12, 2024

(54) BIOLOGICAL VALVE MATERIAL, AND PREPARATION METHOD THEREFOR AND USE THEREOF

(71) Applicant: SICHUAN UNIVERSITY, Chengdu (CN)

(72) Inventors: Yunbing Wang, Chengdu (CN); Cheng Zheng, Chengdu (CN); Li Yang, Chengdu (CN); Yang Lei, Chengdu (CN); Gaocan Li, Chengdu (CN); Rifang Luo, Chengdu (CN); Dajun Kuang, Chengdu (CN); Caili Ma, Chengdu (CN)

(21) Appl. No.: 18/666,839

(22) Filed: May 17, 2024

Related U.S. Application Data
(63) Continuation of application No. PCT/CN2022/132374, filed on Nov. 16, 2022.

(30) Foreign Application Priority Data
Nov. 17, 2021 (CN) 202111365209.4
Nov. 17, 2021 (CN) 202111365214.5
Mar. 18, 2022 (CN) 202210272385.1
Mar. 18, 2022 (CN) 202210273134.5
Mar. 18, 2022 (CN) 202210273141.5
Mar. 18, 2022 (CN) 202210273149.1
Mar. 18, 2022 (CN) 202210273157.6
Nov. 15, 2022 (CN) 202211431077.5

(51) Int. Cl. A61L 27/36; A61F 2/24

(52) U.S. Cl. CPC A61L 27/3641; A61F 2/2415; A61F 2/2418; A61L 27/3633; A61L 27/3687; A61L 2430/20

(57) ABSTRACT

A biological valve material and preparation method therefor and use thereof are provided. The preparation method includes step S100: chemically grafting first carbon-carbon double bonds with amino groups on the biological material, in which at least an aldehyde-based cross-linking is present; step S200: performing polymerization of carbon-carbon double bonds under an action of an initiator to obtain the biological valve material. This method forms more and larger polymer cross-linked networks through dual cross-linking, improving the cross-linking degree and anti-calcification performance of the biological valve material. While introducing carbon-carbon double bonds, additional functional groups are introduced to endow the biological valve material with new characteristics and further improve the performance.

### CROSS-REFERENCE TO RELATED APPLICATIONS

The present application is a Continuation application of PCT Application No. PCT/CN2022/132374, filed on Nov. 16, 2022, which claims the priorities of Chinese Patent Application No. 202111365214.5, filed on Nov. 17, 2021, Chinese Patent Application No. 202111365209.4, filed on Nov. 17, 2021, Chinese Patent Application No. 202210272385.1, filed on Mar. 18, 2022, Chinese Patent Application No. 202210273134.5, filed on Mar. 18, 2022, Chinese Patent Application No. 202210273141.5, filed on Mar. 18, 2022, Chinese Patent Application No. 202210273149.1, filed on Mar. 18, 2022, Chinese Patent Application No. 202210273157.6, filed on Mar. 18, 2022, Chinese Patent Application No. 202211431077.5, filed on Nov. 15, 2022, the entire contents of which are hereby incorporated by reference.

### TECHNICAL FIELD

The present invention relates to the technical field of interventional materials, and in particular to biological valve materials and preparation method therefor and use thereof.

### DESCRIPTION OF THE PRIOR ART

Biological heart valves are usually made from pig or cattle pericardium and are used to replace the human body's native heart valves with functional deficiencies. Biological heart valves have many advantages over mechanical heart valves: patients do not need to take anticoagulants for a long time after the biological heart valves are implanted, and biological heart valves can be applied in minimally invasive interventional surgeries. These advantages have made biological heart valves gradually become the mainstream on the market in clinical applications.

Almost all existing biological valve products on the market are prepared by cross-linking with glutaraldehyde. Glutaraldehyde can cross-link the collagen in the pericardium, thereby enhancing the mechanical properties of the valve to meet market requirements. However, the glutaraldehyde cross-linked biological valve has aldehyde groups for creating calcification sites, and thus has the disadvantage of poor blood compatibility, resulting in a limited lifespan in the human body.

Due to the objective problems of traditional glutaraldehyde cross-linked biological valves, in recent years, there have been some research reports on exploring non-glutaraldehyde cross-linked biological valves. For example, carbodiimide was used as a cross-linking agent to prepare non-glutaraldehyde cross-linked biological valves. However, the research found that the mechanical properties of non-glutaraldehyde cross-linked biological valves are difficult to meet the requirements, and it is difficult to achieve industrialization.

Therefore, glutaraldehyde cross-linked biological valves are still the mainstream materials for biological valves, and improving the performance of the glutaraldehyde cross-linked valves is still one of the current important research directions. Traditionally, the mechanical properties of the valve are improved by increasing the cross-linking time and concentration of glutaraldehyde. However, due to the self-polymerization reaction of glutaraldehyde, the degree of cross-linking is limited, and it is not possible to achieve cross-linking of all amino groups of the valve. Further, blindly increasing the time and concentration will enhance the self-polymerization of the glutaraldehyde on the valve surface, causing the valve to harden.

### SUMMARY OF THE DISCLOSURE

The present disclosure discloses biological valve materials and preparation method therefor and use thereof. Carbon-carbon double bonds are introduced as the basis for secondary cross-linking on the basis of glutaraldehyde cross-linking, and polymerization of carbon-carbon double bonds is further initiated to achieve the secondary cross-linking, thereby improving the performance of glutaraldehyde cross-linked valves.

For the process of introducing carbon-carbon double bonds as the basis for secondary cross-linking, in one aspect, double-bond monomers can be introduced in the glutaraldehyde cross-linking process for co-crosslinking, by introducing other cross-linking groups for cross-linking, a controllable cross-linking can be provided; in another aspect, after glutaraldehyde cross-linking, through the residual amino groups of the glutaraldehyde cross-linked valve, monomers with double bonds can be introduced, by introducing other cross-linking groups for cross-linking, a controllable cross-linking can be provided.

A method for preparing biological valve material, including:
step S100: treating a biological material with a first treatment solution and a second treatment solution in sequence to obtain a pretreated biological material chemically grafted with first carbon-carbon double bonds; wherein the first treatment solution and the second treatment solution respectively contain one of reagent A and reagent B, reagent A is a first functional monomer with a first carbon-carbon double bond, and reagent B is an aldehyde-based cross-linking agent; and
step S200: performing polymerization of carbon-carbon double bonds under an action of an initiator to obtain the biological valve material.

Optionally, in step S100, the first functional monomer further has an active group for participating in chemical reaction; the first treatment solution contains reagent A, and the active group can react with an aldehyde group.

Alternatively, the first treatment solution contains reagent B, and the active group can react with an amino group.

Optionally, the biological material is animal tissue, including one or more of pericardium, valve, intestinal membrane, meninges, pleura, blood vessel, skin or ligament.

Optionally, the animal tissue is a fresh animal tissue or a decellularized biological tissue.

Optionally, the aldehyde-based cross-linking agent is glutaraldehyde or formaldehyde.

In step S200, optionally, the initiator is added to the system treated in the previous step; or the biological material treated in the previous step is taken out and directly soaked in a solution containing the initiator or soaked in a solution containing the initiator after cleaning;

Optionally, the initiator is a single initiator or a mixed initiator, and the mixed initiator is a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, the concentration of each component in the mixture is in the range of 1 to 100 mM.

Alternatively, the mixed initiator is a mixture of ammonium persulfate and N,N,N′,N′-tetramethylethylenediamine, or a mixture of potassium persulfate and N,N,N′,N′-tetramethylethylenediamine, or a mixture of ammonium persulfate and N,N,N′,N′-tetramethylethylenediamine, or a mixture of sodium persulfate and N,N,N′,N′-tetramethylethylenediamine. The mass percentage concentrations of ammonium persulfate, potassium persulfate or sodium persulfate are in the range of 2% to 5% respectively; the mass percentage of tetramethylethylenediamine is in the range of 0.2% to 0.5%.

The single initiator is any component in the mixed initiator.

Optionally, step S100 includes:
AS110: contacting a biological material with a first treatment solution for physical permeation, wherein the first treatment solution is a solution containing the a functional monomer;
AS120: contacting the biological material treated by AS110 with a second treatment solution to perform co-crosslinking and introduce the first carbon-carbon double bonds, wherein the second treatment solution is an aldehyde-based cross-linking agent solution.

Optionally, the active group is amino group or hydrazide.

Optionally, step S100 includes:
BS110: contacting a biological material with a first treatment solution for cross-linking, wherein the first treatment solution is an aldehyde-based cross-linking agent solution;
BS120: contacting the biological material treated in step BS110 with a second treatment solution for chemical reaction to introduce the first carbon-carbon double bonds, wherein the second treatment solution is a solution containing the first functional monomer.

Optionally, the active group is an oxirane group.

Optionally, in step S100, a non-condensation chemical reaction is performed to graft the first carbon-carbon double bonds.

Optionally, in step S100, the biological material does not undergo any chemical reaction involving other reagents before being treated with the aldehyde-based cross-linking agent.

Optionally, in the reaction system of step S100, the first carbon-carbon double bond is provided by the first functional monomer with an active group, and the reaction raw materials in step S100 only include the biological material, the first functional monomer and the aldehyde-based cross-linking agent.

Optionally, step S100 includes:
AS110: soaking a biological material in a first treatment solution for physical permeation, wherein the first treatment solution is a solution containing the first functional monomer, the first functional monomer further has an active group, and the activity group is amino group or hydrazide;
AS120: soaking the biological material treated in step AS110 in a second treatment solution for co-crosslinking to introduce first carbon-carbon double bonds, wherein the second treatment solution is an aldehyde-based cross-linking agent solution.

Optionally, the first functional monomer further includes a functional group A.

Optionally, the functional group A is selected from at least one of hydroxyl group, carboxyl group, amide group, sulfonic acid group, zwitterion, polyethylene glycol, urea group, carbamate group, carboxylate ion, sulfonate, sulfoxide, and pyrrolidone.

Optionally, step S100 further includes:
AS130: soaking the biological material treated in step AS120 into a solution containing a second functional monomer for physical permeation to introduce second carbon-carbon double bonds, wherein the second functional monomer has a second carbon-carbon double bond.

Optionally, the second functional monomer further includes a functional group B.

Optionally, the functional group B is selected from at least one of hydroxyl group, carboxyl group, choline carboxylate, choline sulfonate, choline phosphate, pyrrolidone, sulfonic acid group, carboxylate ion, sulfonate, sulfoxide, amide group and methoxy group.

Optionally, the second functional monomer is selected from at least one of polyethylene glycol diacrylate, 1,4-butanediol diacrylate, ethyl acrylate, N-methyl-2-acrylamide, N-2,2-propenyl-2-acrylamide, N-ethylacrylamide, N,N′-ethylenebisacrylamide, (ethane-1,2-diylbis(oxy))bis(ethane-2,1-diyl)diacrylate, double-bonded hyaluronic acid, acrylamide, 2-(prop-2-enoylamino)acetic acid, 2-acrylamido-2-methylpropanesulfonic acid, hydroxyethyl methacrylate, 3-[[2-(methacryloyloxy)ethyl]dimethylammonio]propionate, N-isopropylacrylamide, N-(hydroxymethyl)acrylamide, N-(2-hydroxyethyl)methacrylamide, N,N-dimethylmethacrylamide, 3-[N,N-dimethyl-[2-(2-methylprop-2-enoyloxy)ethyl]ammonium]propane-1-sulfonate, 2-methacryloyloxyethyl phosphorylcholine and double-bonded polylysine.

Optionally, in step AS130, the second functional monomer is added to the system treated in the previous step; or the biological material treated in the previous step is washed and then soaked in a solution containing a second functional monomer; the solution of the second functional monomer only includes the second functional monomer and a solvent that does not participate in the chemical reaction.

Optionally, the solvent in the solution containing the second functional monomer is water, physiological saline, a neutral pH buffer or an aqueous solution of ethanol; the mass percentage concentration of the second functional monomer in the solution containing the second functional monomer is in the range of 1 to 10%; the soaking time is in the range of 2 to 20 hours.

Optionally, the first functional monomer is selected from at least one of DL-2-amino-4-pentenoic acid, 2-methylallylamine, 3-butene-1-amine, 4-penten-1-amine, 2-aminoethyl methacrylate, methacryloyl hydrazide, acryloyl hydrazide, double bonded polylysine, 2-amino-7-en-octanoic acid, 6-en-aminoheptanoic acid, 2-aminopent-4-enoic acid, 4-(1-amino-2-methyl-propyl)-hepta-1,6-diene-4-ol, and 4-(1-amino-ethyl)-hepta-1,6-diene-4-ol.

Optionally, the solution containing the first functional monomer only includes the first functional monomer and a solvent that does not participate in the chemical reaction.

Optionally, in step AS110, the solvent in the solution containing the first functional monomer is water, physiological saline, isopropanol, a neutral pH buffer or an aqueous solution of ethanol; the concentration of the first functional monomer in the solution containing the first functional monomer is in the range of 10 to 100 mM; the soaking time is in the range of 2 to 20 h.

Optionally, in step AS120, the final concentration of the aldehyde-based cross-linking agent in the AS120 reaction system is in the range of 10 to 800 mM; the co-crosslinking time is in the range of 10 to 30 h.

Optionally, in step S100:
after step AS120, step AS120(M) is further included: soaking the biological material treated in step AS120 in a solution containing a third functional monomer to eliminate residual aldehyde groups; the third functional monomer has an amino group or a hydrazide.

Optionally, in step S100:
before step AS130, step AS120(M) is further included: soaking the biological material treated in step AS120 in a solution containing a third functional monomer to eliminate residual aldehyde groups; the third functional monomer has an amino group or a hydrazide.

Optionally, in step AS120(M), the solvent in the solution containing the third functional monomer is water, physiological saline, a neutral pH buffer or an aqueous solution of ethanol; the concentration of the third functional monomer in the solution containing the third functional monomer is in the range of 10 to 100 mM; the soaking time is in the range of 2 to 48 h.

Optionally, the third functional group further includes a functional group C.

Optionally, the functional group C is selected from at least one of hydroxyl group, carboxyl group, amide group, sulfonic acid group, zwitterion, polyethylene glycol, urea group, carbamate group, carboxylate ion, sulfonate, sulfoxide, and pyrrolidone.

Optionally, the third functional monomer is selected from at least one of DL-2-amino-4-pentenoic acid, 2-methylallylamine, 3-butene-1-amine, 4-penten-1-amine, 2-aminoethyl methacrylate, methacryloyl hydrazide, acryloyl hydrazide, double bonded polylysine, 2-amino-7-en-octanoic acid, 6-en-aminoheptanoic acid, 2-aminopent-4-enoic acid, 4-(1-amino-2-methyl-propyl)-hepta-1,6-diene-4-ol, and 4-(1-amino-ethyl)-hepta-1,6-diene-4-ol.

Optionally, step S100 includes:
BS110: contacting a biological material with a first treatment solution for cross-linking, wherein the first treatment solution is an aldehyde-based cross-linking agent solution;
BS120: soaking the biological material treated in step BS110 in a second treatment solution for chemical reaction to introduce first carbon-carbon double bonds; the second treatment solution is a solution containing the first functional monomer; the first functional monomer further includes an active group; the active group is an oxirane group.

Optionally, step S100 includes:
BS110: contacting a biological material with a first treatment solution for cross-linking, wherein the first treatment solution is an aldehyde-based cross-linking agent solution;
BS120: soaking the biological material treated in step BS110 in a second treatment solution for chemical reaction to introduce first carbon-carbon double bonds; the second treatment solution is a solution containing the first functional monomer; the first functional monomer further includes an active group; the active group is an oxirane group;
BS130: soaking the biological material treated in step BS120 into a solution containing a second functional monomer, where the second functional monomer has a second carbon-carbon double bond.

Optionally, the second functional monomer is one or more of polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-ethylenebisacrylamide, (ethane-1,2-diylbis(oxy))bis(ethane-2,1-diyl)diacrylate, N,N′-dimethylacrylamide, N,N-dimethylmethacrylamide, and double bonded polylysine.

Optionally, the second functional monomer further includes a functional group B.

Optionally, the functional group B is selected from at least one of hydroxyl group, carboxyl group, choline carboxylate, choline sulfonate, choline phosphate, pyrrolidone, sulfonic acid group, carboxylate ion, sulfonate, sulfoxide, amide group and methoxy group.

Optionally, the second functional monomer is one or more of acrylamide, acrylic acid, sodium acrylate, methacrylic acid, sodium methacrylate, 2-(prop-2-enoylamino)acetic acid, 2-acrylamido-2-methylpropanesulfonic acid, hydroxyethyl methacrylate, 3-[[2-(methacryloyloxy)ethyl]dimethylammonio]propionate, N-methyl-2-acrylamide, N-isopropylacrylamide, N-(hydroxymethyl)acrylamide, N-(2-hydroxyethyl)methacrylamide, 3-[N,N-dimethyl-[2-(2-methylprop-2-enoyloxy)ethyl]ammonium]propane-1-sulfonate, 2-methacryloyloxyethyl phosphorylcholine, N-(2-hydroxyethyl)acrylamide, N-(methoxymethyl)methacrylamide, 2-acrylamido-2-methylpropanesulfonic acid, and double-bonded hyaluronic acid.

In step BS130, optionally, the second functional monomer enters the biological material through physical permeation.

The physical permeation refers to the process wherein the biological material treated in step S120 is soaked in a solution containing the second functional monomer. During this process, the second functional monomer in the solution adheres to the surface of the biological material or embeds into the gaps of the biological material, without undergoing any chemical reaction with the biological material.

Optionally, the solution containing the second functional monomer only includes the second functional monomer and a solvent that does not participate in the reaction.

Optionally, the v/v concentration of the second functional monomer in the solution containing the second functional monomer is in the range of 0.1% to 20%; the soaking time is in the range of 0.5 h to 120 h.

Further, the v/v concentration of the second functional monomer in the solution containing the second functional monomer is in the range of 0.1% to 6%.

Optionally, the first functional monomer is selected from at least one of allyl glycidyl ether, glycidyl methacrylate and glycidyl acrylate.

Optionally, in step BS110:
the w/w concentration of the aldehyde-based cross-linking agent solution is in the range of 0.1% to 5%; the cross-linking time is in the range of 0.5 h to 120 h.

Optionally, in step BS120:
the solution containing the first functional monomer only includes the first functional monomer and a solvent that does not participate in the chemical reaction.

Optionally, the w/w concentration of the first functional monomer in the solution containing the first functional monomer is in the range of 1% to 10%; the reaction time is in the range of 2 to 120 hours.

Optionally, the solvent in the solution containing the first functional monomer is one or more of an aqueous solution of any one of methanol, ethanol, ethylene glycol, propanol, 1,2-propanediol, 1,3-propanediol, isopropanol, butanol, isobutanol, 1,2-butanediol, 1,3-butanediol, 1,4-butanediol, and glycerol, water, physiological saline, and a neutral pH buffer.

The present disclosure further provides a method for preparing biological valve material, including:
step BS110: contacting a biological material with an aldehyde-based cross-linking agent solution for cross-linking;
step BS120: soaking the biological material treated in step BS110 into a solution containing a first functional monomer for reaction to introduce first carbon-carbon double bonds; wherein the first functional monomer has the first carbon-carbon double bond and an oxirane group;
step S200: performing polymerization of carbon-carbon double bonds under the action of an initiator to obtain a biological valve material.

The present disclosure further provides a method for preparing biological valve material, including:
step BS110: contacting a biological material with an aldehyde-based cross-linking agent solution for cross-linking;
step BS120: soaking the biological material treated in step BS110 into a solution containing a first functional monomer for reaction to introduce first carbon-carbon double bonds; wherein the first functional monomer has the first carbon-carbon double bond and an oxirane group;
step BS130: soaking the biological material treated in step BS120 in a solution containing a second functional monomer, wherein the second functional monomer has a second carbon-carbon double bond; and
step S200: performing polymerization of carbon-carbon double bonds under the action of an initiator to obtain a biological valve material.

The present disclosure further provides a method for preparing biological valve material, including:
step BS110: contacting a biological material with an aldehyde-based cross-linking agent solution for cross-linking;
step BS120: soaking the biological material treated in step BS110 into a solution containing a first functional monomer for reaction to introduce first carbon-carbon double bonds; wherein the first functional monomer has the first carbon-carbon double bond and an oxirane group;
step BS130: soaking the biological material treated in step BS120 in a solution containing a second functional monomer, wherein the second functional monomer has a second carbon-carbon double bond and a functional group B; and
step S200: performing polymerization of carbon-carbon double bonds under the action of an initiator to obtain a biological valve material.

The present disclosure further provides a biological valve material prepared by the above preparation method.

The present disclosure further provides a biological valve material, including:
step S100: treating the biological material with a first treatment solution and a second treatment solution in sequence to obtain a pretreated biological material chemically grafted with first carbon-carbon double bonds; wherein the first treatment solution and the second treatment solution respectively contain one of reagent A and reagent B, reagent A is a first functional monomer with a first carbon-carbon double bond, and reagent B is an aldehyde-based cross-linking agent; and
step S200: performing polymerization of carbon-carbon double bonds under an action of an initiator to obtain the biological valve material.

The present disclosure further provides a biological valve material, including:
step BS110: contacting a biological material with an aldehyde-based cross-linking agent solution for cross-linking;
step BS120: soaking the biological material treated in step BS110 into a solution containing a first functional monomer for reaction to introduce first carbon-carbon double bonds; wherein the first functional monomer has the first carbon-carbon double bond and an oxirane group;
step S200: performing polymerization of carbon-carbon double bonds under the action of an initiator to obtain a biological valve material.

The present disclosure further provides a biological valve material, including:
step BS110: contacting a biological material with an aldehyde-based cross-linking agent solution for cross-linking;
step BS120: soaking the biological material treated in step BS110 into a solution containing a first functional monomer for reaction to introduce first carbon-carbon double bonds; wherein the first functional monomer has the first carbon-carbon double bond and an oxirane group;
step BS130: soaking the biological material treated in step BS120 in a solution containing a second functional monomer, wherein the second functional monomer has a second carbon-carbon double bond; and
step S200: performing polymerization of carbon-carbon double bonds under the action of an initiator to obtain a biological valve material.

The present disclosure further provides a biological valve material, including:
step BS110: contacting a biological material with an aldehyde-based cross-linking agent solution for cross-linking;
step BS120: soaking the biological material treated in step BS110 into a solution containing a first functional monomer for reaction to introduce first carbon-carbon double bonds; wherein the first functional monomer has the first carbon-carbon double bond and an oxirane group;
step BS130: soaking the biological material treated in step BS120 in a solution containing a second functional monomer, wherein the second functional monomer has a second carbon-carbon double bond and a functional group B; and
step S200: performing polymerization of carbon-carbon double bonds under the action of an initiator to obtain a biological valve material.

The present disclosure further provides a biological valve, including a stent and leaflets, and the leaflets are the biological valve material.

Optionally, the biological valve is a heart valve.

The present disclosure further provides an interventional system, including a heart valve and a catheter assembly. The heart valve is collapsed and delivered by the catheter assembly. The heart valve includes a stent and leaflets, and the leaflets are the biological valve material.

Compared with the prior art, the present disclosure has at least one of the following benefits:

(1) The method of the present disclosure introduces functional monomers for co-crosslinking during aldehyde cross-linking. During co-crosslinking, carbon-carbon double bonds are introduced as the basis for secondary cross-linking. Through dual cross-linking, biological valve materials can be prepared with improved cross-linking degree and mechanical properties.

(2) In the co-crosslinking process of the present disclosure, the functional monomers introduce double bonds and can also block some residual aldehyde groups on the biological material, improving the anti-calcification and anti-coagulation properties of the biological material, and also further improving the cross-linking degree.

(3) The method of the present disclosure can also introduce functional groups while introducing carbon-carbon double bonds, which can further improve the properties of the biological material, such as surface hydrophilicity, biocompatibility, etc.

(4) After the co-crosslinking is completed, the biological material is soaked in the functional monomer solution again to eliminate the remaining residual aldehyde groups and introduce carbon-carbon double bonds, thereby providing more carbon-carbon double bonds for subsequent double bond polymerization and further improving the cross-linking degree of the biological material.

(5) The present disclosure introduces second functional monomers for copolymerization in the double bond polymerization step to form more and larger polymer cross-linked networks, thereby improving the cross-linking degree of the biological material and improving the anti-calcification performance.

(6) The present disclosure not only introduces carbon-carbon double bonds secondarily, but also introduces additional functional groups, which can give new characteristics to the biological material and further improve the performance of the biological material.

(7) During glutaraldehyde cross-linking, the biological material is co-crosslinked with functional monomers to improve the anti-calcification and anti-coagulation properties of the glutaraldehyde cross-linked biological material.

The scheme, in which the biological material is first cross-linked with glutaraldehyde and then introduced with carbon-carbon double bonds, and finally a polymerization of carbon-carbon double bonds is initiated for secondary cross-linking, has further benefits:

(8) Based on the biological valve material cross-linked by glutaraldehyde, the present disclosure introduces double bonds to the glutaraldehyde cross-linked biological valve material through double bonding modification as the basis for secondary cross-linking, and further a polymerization of the double bonds on the glutaraldehyde cross-linked biological valve material is initiated for secondary cross-linking, which can further increase the cross-linking degree of the biological valve material, thereby improving the stability of the biological valve material, and can further reduce the risk of calcification caused by structural degradation, thereby improving the anti-calcification properties.

(9) In the scheme of first performing cross-linking with glutaraldehyde and then chemically grafting the first carbon-carbon double bonds, the functional monomers with carbon-carbon double bonds are chemically linked with the amino, hydroxyl and carboxyl groups on the surface of the glutaraldehyde cross-linked valve through the oxirane groups. The carbon-carbon double bonds are mainly connected to the surface of the biological valve material, which can better protect the fibril structure of the biological material, maintaining the original orientation direction of the fibers of the biological material while effectively ensuring the mechanical properties of the valve.

### BRIEF DESCRIPTION OF DRAWINGS

FIG. 1 is a process flow diagram of a preferred embodiment of Scheme 1 of the present disclosure;

FIG. 2 is a reaction schematic diagram of an embodiment of Scheme 1 of the present disclosure;

FIG. 3 is a process flow diagram of a preferred embodiment of Scheme 2 of the present disclosure;

FIG. 4 is a process flow diagram of a preferred embodiment of Scheme 3 of the present disclosure;

FIG. 5 is a process flow diagram of a preferred embodiment of Scheme 4 of the present disclosure;

FIG. 6 is a process flow diagram of a preferred embodiment of Scheme 5 of the present disclosure;

FIG. 7 is a reaction schematic diagram of a preferred embodiment of the present disclosure;

FIG. 8 is a reaction schematic diagram of another preferred embodiment of the present disclosure;

FIG. 9 is a graph showing infrared spectra of the pericardium (GA) of sample 1 in Example 1 and control group 1;

FIG. 10 is a graph showing the lactate dehydrogenase relative activity results of the pericardium (GA) of sample 1 in Example 1 and control group 1;

FIG. 11 is a graph showing the calcium content results of the pericardium (GA) of sample 1 in Example 1 and control group 1 after subcutaneous implantation in rats;

FIG. 12 is a graph showing the water contact angle test results of the pericardium (GA) of sample 2 in Example 2 and control group 2;

FIG. 13 is a graph showing the results of lactate dehydrogenase detection and hemolysis rate of the pericardium (GA) of sample 2 in Example 2 and control group 2;

FIG. 14 is a graph showing the calcium ion concentration results of the pericardium (GA) of sample 2 in Example 2 and control group 2;

FIG. 15 is a basic schematic diagram in Example 3;

FIG. 16 shows the results of alizarin red staining of slices of the control group 3;

FIG. 17 is an image showing the alizarin red staining result of the slice of sample 3 after subcutaneous implantation in rat;

FIG. 18 is an image showing the alizarin red staining result of the slice of sample 4 after subcutaneous implantation in rat;

FIG. 19 is an image showing the alizarin red staining result of the slice of sample 5 after subcutaneous implantation in rat;

FIG. 20 is an image showing the alizarin red staining result of the slice of sample 6 after subcutaneous implantation in rat;

FIG. 21 is an image showing the alizarin red staining result of the slice of sample 7 after subcutaneous implantation in rat;

FIG. 22 is an image showing the alizarin red staining result of the slice of sample 8 after subcutaneous implantation in rat;

FIG. 23 is a basic schematic diagram in Example 10;

FIG. 24 is an image showing the alizarin red stained slice of the control sample in control group 4 after implantation for 30 days;

FIG. 25 is an image showing the alizarin red stained slice of sample 10 after implantation for 30 days;

FIG. 26 is an image showing the alizarin red stained slice of sample 11 after implantation for 30 days;

FIG. 27 is an image showing the alizarin red stained slice of sample 12 after implantation for 30 days;

FIG. 28 is an image showing the alizarin red stained slice of sample 13 after implantation for 30 days;

FIG. 29 is an image showing the alizarin red stained slice of sample 14 after implantation for 30 days;

FIG. 30 is an image showing the alizarin red stained slice of sample 15 after implantation for 30 days;

FIG. 31 is a scanning electron microscope image of the blood contact experiment of control sample 4;

FIG. 32 is a scanning electron microscope image of the blood contact experiment of sample 12;

FIG. 33 is a scanning electron microscope image of the blood contact experiment of sample 13;

FIG. 34 is a basic schematic diagram in Example 17;

FIG. 35 is an image showing the alizarin red stained slice of the control sample in control group 5 after implantation for 30 days;

FIG. 36 is an image showing the alizarin red stained slice of sample 17 after implantation for 30 days;

FIG. 37 is an image showing the alizarin red stained slice of sample 18 after implantation for 30 days;

FIG. 38 is an image showing the alizarin red stained slice of sample 19 after implantation for 30 days;

FIG. 39 is an image showing the alizarin red stained slice of sample 20 after implantation for 30 days;

FIG. 40 is an image showing the alizarin red stained slice of sample 21 after implantation for 30 days;

FIG. 41 is a basic schematic diagram in Example 23;

FIG. 42 is a scanning electron microscope image of the blood of the control sample in control group 6;

FIG. 43 is a scanning electron microscope image of the blood incubation experiment of sample 23;

FIG. 44 is a scanning electron microscope image of the blood incubation experiment of sample 24;

FIG. 45 is an image showing the alizarin red stained slice of the control sample in control group 6 after implantation for 60 days;

FIG. 46 is an image showing the alizarin red stained slice of sample 23 after implantation for 60 days;

FIG. 47 is an image showing the alizarin red stained slice of sample 24 after implantation for 60 days;

FIG. 48 is an image showing the alizarin red stained slice of sample 25 after implantation for 60 days;

FIG. 49 is an image showing the alizarin red stained slice of sample 26 after implantation for 60 days;

FIG. 50 is an image showing the alizarin red stained slice of sample 27 after implantation for 60 days;

FIG. 51 is a reaction schematic diagram in Examples 29 and 30;

FIG. 52 is a reaction schematic diagram in Example 31;

FIG. 53 is a reaction schematic diagram in Example 32;

FIG. 54 is a reaction schematic diagram in Example 33;

FIG. 55 is a scanning electron microscope image of the blood contact experiment of control sample 7;

FIG. 56 is a scanning electron microscope image of the blood contact experiment of sample 29;

FIG. 57 is a scanning electron microscope image of the blood contact experiment of sample 31;

FIG. 58 is an image showing the alizarin red stained slice of control sample 7 after implantation in rat for 30 days;

FIG. 59 is an image showing the alizarin red stained slice of sample 30 after implantation in rat for 30 days;

FIG. 60 is an image showing the alizarin red stained slice of sample 32 after implantation in rat for 30 days;

FIG. 61 is an image showing the alizarin red stained slice of sample 33 after implantation in rat for 30 days;

FIG. 62 is an image showing the alizarin red stained slice of sample 34 after implantation in rat for 30 days;

FIG. 63 is a process flow diagram of a preferred embodiment of Scheme 6 of the present disclosure;

FIG. 64 is a reaction schematic diagram of a preferred embodiment of Scheme 6 of the present disclosure;

FIG. 65 is an image showing the alizarin red staining result of control group 8 (glutaraldehyde cross-linked porcine pericardium) after subcutaneous implantation in rat for 30 days;

FIG. 66 is a diagram showing the results of alizarin red staining of sample 42 in Example 42 after subcutaneous implantation in rat for 30 days;

FIG. 67 is a diagram showing the results of alizarin red staining of sample 46 in Example 46 after subcutaneous implantation in rat for 30 days;

FIG. 68 is a diagram showing the results of alizarin red staining of sample 48 in Example 48 after subcutaneous implantation in rat for 30 days;

FIG. 69 is a process flow diagram of the double-bond post-copolymerization and cross-linking embodiment of Scheme 7 of the present disclosure;

FIG. 70 is a reaction schematic diagram of the double-bond post-copolymerization and cross-linking embodiment of Scheme 7 of the present disclosure;

FIG. 71 is an image showing the alizarin red staining result of control group 9 (glutaraldehyde cross-linked porcine pericardium) after subcutaneous implantation in rat for 30 days;

FIG. 72 is an image showing the alizarin red staining result of sample 52 in Example 52 after subcutaneous implantation in rat for 30 days;

FIG. 73 is an image showing the alizarin red staining result of sample 53 in Example 53 after subcutaneous implantation in rat for 30 days;

FIG. 74 is an image showing the alizarin red staining result of sample 56 in Example 56 after subcutaneous implantation in rat for 30 days;

FIG. 75 is a process flow diagram of the double-bond post-functionalized copolymerization and cross-linking embodiment of Scheme 8 of the present disclosure;

FIG. 76 is a reaction schematic diagram of the double-bond post-functionalized copolymerization and cross-linking embodiment of Scheme 8 of the present disclosure;

FIG. 77 is a scanning electron microscope image of blood adhesion in control group 10 (glutaraldehyde cross-linked porcine pericardium);

FIG. 78 is a scanning electron microscope image of blood adhesion of sample 62 in Example 62;

FIG. 79 is a scanning electron microscope image of blood adhesion of sample 63 in Example 63;

FIG. 80 is a scanning electron microscope image of blood adhesion of sample 68 in Example 68;

FIG. 81 is an image showing the alizarin red staining result of control group 10 (glutaraldehyde cross-linked porcine pericardium) after subcutaneous implantation in rat for 30 days;

FIG. 82 is an image showing the alizarin red staining result of sample 62 in Example 62 after subcutaneous implantation in rat for 30 days;

FIG. 83 is an image showing the alizarin red staining result of sample 63 in Example 63 after subcutaneous implantation in rat for 30 days;

FIG. 84 is an image showing the alizarin red staining result of sample 69 in Example 69 after subcutaneous implantation in rat for 30 days;

FIG. 85 is a schematic structural view of a heart valve of the present disclosure; and

FIG. 86 is a schematic structural view of an interventional system of the present disclosure.

### DESCRIPTION OF EMBODIMENTS

The technical solutions according to the embodiments of the present disclosure will be described clearly and fully in combination with the drawings according to the embodiments of the present disclosure. Obviously, the described embodiments are not all embodiments of the present disclosure, but only part of the embodiments of the present disclosure. Based on the disclosed embodiments, all other embodiments obtained by those skilled in the art without creative work fall into the scope of this invention.

Unless otherwise defined, all technical and scientific terms used herein have the same meaning as commonly understood by a person skilled in the art. The terms in the description of the present disclosure are used to describe specific embodiments, and not to limit the present disclosure.

In order to improve the performance of conventional glutaraldehyde cross-linked valves, in addition to cross-linking with glutaraldehyde, here, secondary cross-linking of carbon-carbon double bonds is induced by introducing additional carbon-carbon double bonds, thereby improving anti-coagulation, anti-calcification, elasticity and other properties of the glutaraldehyde cross-linked biological valves. Specifically, a method for preparing a biological valve material is provided, including:
step S100: treating the biological material with a first treatment solution and a second treatment solution in sequence to obtain a pretreated biological material chemically grafted with first carbon-carbon double bonds; wherein the first treatment solution and the second treatment solution respectively contain one of reagent A and reagent B, reagent A is a first functional monomer with a first carbon-carbon double bond, and reagent B is an aldehyde-based cross-linking agent; and
step S200: performing polymerization of carbon-carbon double bonds under an action of an initiator to obtain the biological valve material.

The first carbon-carbon double bonds introduced through a chemical reaction undergo polymerization under the action of the initiator to further form a cross-linked network, improving the anti-coagulation, anti-calcification, elasticity and other properties of the glutaraldehyde cross-linked biological valve.

The biological materials used here are conventional biological materials used in the existing glutaraldehyde cross-linking processes, with collagen content ranging from 60% to 90%. Further, the biological materials are animal tissues originated from pig, cattle, horse or sheep, including one or more of pericardium, valve, intestinal membrane, meninges, pleura, blood vessel, skin or ligament.

Optionally, the animal tissues are fresh animal tissues or decellularized biological tissues.

Optionally, in the decellularization, the biological tissues are treated with a surfactant as follows:
the biological tissues undergo decellularization by using an ionic surfactant; or
the biological tissues undergo decellularization by using a non-ionic surfactant.

The ionic surfactant is mainly used to lyse cells, and the non-ionic surfactant is mainly used to remove lipid substances (such as phospholipids).

Optionally, the ionic surfactant is at least one of sodium deoxycholate, potassium fatty acid soap, sodium dodecyl sulfate, sodium cholate, cetyltrimethylammonium bromide, potassium fatty acid salt, and alkyl dimethyl sulfopropyl betaine.

Optionally, the nonionic surfactant is at least one of Triton and Tween.

Optionally, the ionic surfactant is sodium dodecyl sulfonate, and the nonionic surfactant is Triton.

Optionally, the ionic surfactant is sodium dodecyl sulfonate, and the nonionic surfactant is Tween-20.

The cross-linking agent in the present disclosure is an aldehyde-based cross-linking agent used in current mainstream cross-linking methods. Optionally, the aldehyde-based cross-linking agent is selected from glutaraldehyde and formaldehyde.

Optionally, in step S100, a non-condensation chemical reaction is performed to graft the first carbon-carbon double bonds.

Optionally, in step S100, the biological material does not undergo any chemical reaction involving other reagents before being treated with the aldehyde-based cross-linking agent.

Further optionally, in the reaction system of step S100, the first carbon-carbon double bond is provided by the first functional monomer with an active group, and the reaction raw materials in step S100 only include the biological material, the first functional monomer and the aldehyde-based cross-linking agent. That is, when the first treatment solution contains reagent A (the first functional monomer with the first carbon-carbon double bond), the first treatment solution only contains the first functional monomer and a solvent that does not participate in the chemical reaction; when the first treatment solution contains reagent B (the aldehyde-based cross-linking agent), the first treatment solution only contains the aldehyde-based cross-linking agent and a solvent that does not participate in the chemical reaction. The same applies to the second treatment solution.

In the present disclosure, when treating the biological material with a first treatment solution and a second treatment solution in sequence as mentioned above, the order in which the involved reagents come into contact with the biological material, and the way of adding material or contacting each other are not strictly limited. For example, after being treated with the first treatment solution, the biological material can be taken out and contacted with the second treatment solution, or the second treatment solution can be directly added to the first treatment solution with the biological material soaked therein.

The first treatment solution and the second treatment solution respectively containing one of reagent A and reagent B as mentioned above means that, when the first treatment solution contains reagent A, the second treatment solution contains reagent B, which means that the second treatment solution does not contain reagent A, rather than that the second treatment solution only contains reagent B; similarly, when the first treatment solution contains reagent B, the second treatment solution contains reagent A, which means that the second treatment solution does not contain reagent B, rather than that the second treatment solution only contains reagent A.

In step S100, the first functional monomer needs to participate in the chemical grafting reaction. Optionally, the first functional monomer further has an active group for participating in the chemical grafting reaction.

Further, when the first treatment solution contains reagent A (the first functional monomer with the first carbon-carbon double bond) and the second treatment solution contains reagent B (the aldehyde-based cross-linking agent), the active group of the first functional monomer can react with the aldehyde group, thereby indirectly grafting the first carbon-carbon double bond to the biological material through the chemical reaction. When the first treatment solution contains reagent B (the aldehyde-based cross-linking agent) and the second treatment solution contains reagent A (the first functional monomer with the first carbon-carbon double bond), the active group of the first functional monomer can react with the amino group, thereby directly grafting the first carbon-carbon double bond to the biological material. That is, for chemically grafting the first carbon-carbon double bond in step S100, the first carbon-carbon double bond can be indirectly connected to the biological material through the cross-linking agent, or can be directly connected to the biological material through reaction with the active group (at least including amino group) of the biological material.

In one alternative, in step S100, the first treatment solution contains reagent A (the first functional monomer with the first carbon-carbon double bond), and the second treatment solution contains reagent B (the aldehyde-based cross-linking agent). In this alternative, the first functional monomer first physically permeates into the biological material, and then the amino group of the biological material and the active group of the first functional monomer are co-crosslinked with the aldehyde-based cross-linking agent, thereby grafting the first carbon-carbon double bond. In this alternative, the first functional monomer first physically permeates into the biological material, and then the aldehyde-based cross-linking agent is added to perform co-crosslinking (i.e., the chemical reaction described above). The amino groups on the biological material are indirectly grafted with the first carbon-carbon double bonds through the aldehyde-based cross-linking agent. In this alternative, optionally, the active group of the first functional monomer is an amino group or a hydrazide.

In another alternative, in step S100, the first treatment solution contains reagent B (the aldehyde-based cross-linking agent), and the second treatment solution contains reagent A (the first functional monomer with the first carbon-carbon double bond). In this alternative, the biological material first undergoes a cross-linking reaction with the aldehyde-based cross-linking agent, and then reacts with the active group of the first functional monomer to graft the first carbon-carbon double bond. In this alternative, in step S100, the aldehyde-based cross-linking agent is first added to react with part of the amino groups of the biological material first, and then the first functional monomer is added so that the first carbon-carbon double bond is directly grafted by using the remaining amino groups and other groups (such as hydroxyl and carboxyl groups) on the biological material that react with the active group of the first functional monomer. In this alternative, optionally, the active group of the first functional monomer is an oxirane group. In addition to the remaining amino groups on the biological material participating in the reaction, the hydroxyl and carboxyl groups can also react with the oxirane group and thus participate in the chemical reaction.

In the first alternative, in the glutaraldehyde treatment, the functional monomer added undergoes co-crosslinking, with the carbon-carbon double bonds introduced as the basis for secondary cross-linking. Through dual cross-linking to prepare the cross-linked biological material, the degree of cross-linking of the biological material and thus the mechanical properties of the biological material can be improved. In the second alternative, cross-linking with glutaraldehyde is performed first, and then the active groups on the glutaraldehyde cross-linked valve such as the residual amino groups, hydroxyl groups, and carboxyl groups are chemically connected to the functional monomer with carbon-carbon double bond, wherein the functional monomer with carbon-carbon double bond, through the oxirane group thereof, is connected with the amino groups, hydroxyl groups, and carboxyl groups on the surface of the glutaraldehyde cross-linked valve through chemical reaction, thereby generally grafting the carbon-carbon double bonds to the surface of the biological valve material. In addition to improving the cross-linking degree and mechanical properties of the biological material through dual cross-linking, the fibril structure of the biological material can be better protected, maintaining the original orientation direction of the fibers of the biological material while effectively ensuring the mechanical properties of the valve.

Using the amino groups on the biological material in the present disclosure means that at least part of the amino groups on the biological material participates in the chemical reaction of introducing the first carbon-carbon double bonds. In practice, step S100 can include multiple sub-steps. The raw materials involved in the reaction system of step S100 may participate in at least one of the sub-steps, and are not required to participate in all sub-steps.

In one aspect, the present disclosure develops the existing cross-linking methods based on glutaraldehyde by introducing a functional monomer for co-crosslinking in addition to the glutaraldehyde cross-linking. This enhances the performance of the glutaraldehyde cross-linked biological material and addresses the issues of poor anti-calcification and anti-coagulation properties.

In this developed cross-linking scheme (referred as Scheme 1), a functional monomer with a group reactive with an aldehyde group is introduced before glutaraldehyde cross-linking. The functional monomer first physically permeates into the biological material and then undergoes co-crosslinking with an aldehyde-based cross-linking agent.

Specifically, Scheme 1 includes:
AS110: contacting a biological material with a solution containing a first functional monomer to perform physical permeation, wherein the first functional monomer has at least one group for reacting with an aldehyde group; and
S120: adding an aldehyde-based cross-linking agent to the system in step AS110 to perform co-crosslinking.

The reaction mechanism of Scheme 1:
the functional monomer first physically permeates into the biological material, wherein the functional monomer has the group that can react with the aldehyde group; after the functional monomer permeates into the biological material, the aldehyde-based cross-linking agent is added to perform co-crosslinking, wherein the co-crosslinking at least includes the following reactions:
1) the aldehyde groups at both ends of part of the cross-linking agent react with the amino groups of the biological material; 2) the aldehyde groups at one end of part of the cross-linking agent react with the amino groups of the biological material, while the aldehyde groups at the other end react with the first functional monomer; 3) the aldehyde groups at one end of part of the cross-linking agent reacts with the amino groups of the biological material, while the aldehyde groups at the other end form residual aldehyde groups on the biological material; 4) part of the residual aldehyde groups react with the amino group of the first functional monomer.

In another aspect, almost all existing biological valve products on the market are prepared by cross-linking with glutaraldehyde. Glutaraldehyde can cross-link the collagen in the pericardium. However, the glutaraldehyde cross-linked biological valve involves a thrombosis issue that cannot be ignored and poses a serious threat to the patient's quality of life and survival. The present disclosure develops the cross-linking method based on glutaraldehyde to improve the mechanical properties, anti-calcification and anti-coagulation properties of the glutaraldehyde cross-linked valve.

In this developed cross-linking scheme (referred as Scheme 2), a first functional monomer with a carbon-carbon double bond and a group reactive with an aldehyde group is introduced before glutaraldehyde cross-linking. The first functional monomer first physically permeates into the biological material, and then undergoes co-crosslinking with an aldehyde-based cross-linking agent. During the co-crosslinking process, the first functional monomer reacts with the residual aldehyde groups on the biological material to introduce the first carbon-carbon double bond to the biological material. Then, a secondary cross-linking is induced in which the double bonds undergo polymerization, thereby completing the cross-linking treatment of the biological material. The biological material after secondary cross-linking may also have functional groups to further improve the biocompatibility of the biological valve.

Specifically, Scheme 2 includes:
AS110: soaking a biological material in a solution containing a first functional monomer for physical permeation, wherein the first functional monomer has at least one first carbon-carbon double bond and at least one group for reacting with an aldehyde group;
AS120: adding an aldehyde-based cross-linking agent to the system in step AS110 to perform co-crosslinking; and
S200: contacting the biological material treated in step AS120 with an initiator to initiate double bond polymerization.

The reaction mechanism of Scheme 2:
in the first step, the first functional monomer physically permeates into the biological material first, wherein the introduced first functional monomer has the first carbon-carbon double bond and the group that can react with the aldehyde group (such as amino group); after the functional monomer sufficiently permeates into the biological material, the aldehyde-based cross-linking agent (such as glutaraldehyde) is added to perform co-crosslinking, wherein the co-crosslinking at least includes the following reactions:
1) the aldehyde groups at both ends of part of the cross-linking agent react with the amino groups of the biological material; 2) the aldehyde groups at one end of part of the cross-linking agent react with the amino groups of the biological material, and the aldehyde groups at the other end react with the amino group of the first functional monomer; 3) the aldehyde groups at one end of part of the cross-linking agent react with the amino groups of the biological material, and the aldehyde groups at the other end form residual aldehyde groups on the biological material; 4) part of the residual aldehyde groups react with the amino group of the first functional monomer to introduce the first carbon-carbon double bond to the biological material;
in the second step, the cross-linked biological material with introduced carbon-carbon double bonds is in contact with a solution containing the initiator to initiate double bond polymerization (i.e., the secondary cross-linking).

In a further aspect, in order to further improve the biocompatibility of the glutaraldehyde cross-linked valve, the present disclosure further introduces a functional group to the functional monomer in Scheme 2, that is, in a developed cross-linking scheme (referred as Scheme 3), the first functional monomer with an amino group, a first carbon-carbon double bond and a functional group A is introduced before glutaraldehyde cross-linking. The functional monomer first physically permeates into the biological material, and then undergoes co-crosslinking with an aldehyde-based cross-linking agent, wherein the amino group of the first functional monomer reacts with the aldehyde group, with the first carbon-carbon double bond and the functional group A both introduced to the biological material. Then, a secondary cross-linking is induced in which the double bonds undergo polymerization. The biological material after secondary cross-linking contains functional groups A, which can further improve the biocompatibility of the biological material.

Specifically, Scheme 3 includes:
AS110: soaking a biological material in a solution containing a first functional monomer for physical permeation, wherein the first functional monomer has at least one amino group, at least one first carbon-carbon double bond and at least one functional group A;
AS120: adding an aldehyde-based cross-linking agent to the solution in which the biological material treated in step AS110 is soaked to perform co-crosslinking; and
S200: contacting the biological material treated in step AS120 with an initiator to initiate double bond polymerization.

The reaction mechanism of Scheme 3:
in the first step, the first functional monomer first physically permeates into the biological material, wherein the introduced first functional monomer has the amino group, the first carbon-carbon double bond and the functional group A; after the first functional monomer sufficiently permeates into the biological material, the aldehyde-based cross-linking agent (such as glutaraldehyde) is added to perform co-crosslinking, wherein the co-crosslinking at least includes the following reactions:
1) the aldehyde groups at both ends of part of the cross-linking agent react with the amino groups of the biological material; 2) the aldehyde groups at one end of part of the cross-linking agent react with the amino groups of the biological material, and the aldehyde groups at the other end react with the amino group of the first functional monomer; 3) the aldehyde groups at one end of part of the cross-linking agent react with the amino groups of the biological material, and the aldehyde groups at the other end form residual aldehyde groups on the biological material; 4) part of the residual aldehyde groups react with the amino group of the first functional monomer to introduce the first carbon-carbon double bond to the biological material;
in the second step, the cross-linked biological material with introduced carbon-carbon double bonds is in contact with a solution containing the initiator to initiate the polymerization of the carbon-carbon double bonds on the biological material (i.e., the secondary cross-linking).

The method of this scheme introduces the first functional monomer for co-crosslinking during aldehyde cross-linking. During co-crosslinking, the first carbon-carbon double bond is introduced as the basis for secondary cross-linking. The cross-linked biological material prepared through dual cross-linking has an improved cross-linking degree. The method in the present disclosure not only introduces the first carbon-carbon double bond but also introduces the functional group A, which can further improve the properties of the biological material, such as surface hydrophilicity, biocompatibility, etc.

In a further aspect, in order to further improve the mechanical properties, anti-calcification and anti-coagulation properties of the glutaraldehyde cross-linked valve, the present disclosure further develops the cross-linking method on the basis of Scheme 3.

In this developed cross-linking scheme (referred as Scheme 4), a first functional monomer with a first carbon-carbon double bond and a group reactive with an aldehyde group is introduced before glutaraldehyde cross-linking. The first functional monomer first physically permeate into the biological material, and then undergoes co-crosslinking with an aldehyde-based cross-linking agent, wherein the amino group of the first functional monomer reacts with the aldehyde group, with the first carbon-carbon double bond and the functional group A both introduced to the biological material. Then, second carbon-carbon double bonds are introduced through a physical permeation of a second functional monomer. Finally, the first carbon-carbon double bond of the first functional monomer and the second carbon-carbon double bond of the second functional monomer on the biological material undergo polymerization form a cross-linked network, to further improve the cross-linking degree of the biological material.

Specifically, Scheme 4 includes:
AS110: soaking a biological material in a solution containing a first functional monomer for physical permeation, wherein the first functional monomer has at least one first carbon-carbon double bond and at least one group for reacting with a residual aldehyde group on the biological material;
AS120: adding an aldehyde-based cross-linking agent to the system in step AS110 to perform co-crosslinking;
AS130: soaking the biological material treated in step AS120 into a solution containing a second functional monomer for physical permeation, wherein the second functional monomer has at least one second carbon-carbon double bond; and
S200: adding an initiator to the system in step AS130 to initiate double bond polymerization.

The reaction mechanism of Scheme 4:
the reaction mechanism in the first step is the same as that in the first step of Scheme 3, which will not be repeated again here;
in the second step, the second functional monomer first physically permeates into the biological material which has undergone co-crosslinking with first carbon-carbon double bonds introduced, so as to further introduce second carbon-carbon double bonds by physical permeation; after the second functional monomer permeates into the biological material, a copolymerization is initiated between the second carbon-carbon double bond of the second functional monomer and the first carbon-carbon double bond on the surface of the biological material (i.e. a secondary cross-linking) to form a cross-linked network.

In the method in this scheme, the biological material is cross-linked through aldehyde co-crosslinking and secondary cross-linking of double bond polymerization. The biological material treated by dual cross-linking has a good degree of cross-linking. In this scheme, first carbon-carbon double bonds are introduced through the co-crosslinking, and then second carbon-carbon double bonds are further introduced through the second functional monomer. The second carbon-carbon double bonds are introduced through physical permeation so that a copolymerization can be initiated with the additional functional monomer involved during the double bond polymerization process, forming a larger polymer cross-linked network, which is beneficial to improving the cross-linking degree and anti-calcification performance of the biological valve.

In a further aspect, in order to further improve the performance of the biological material, such as biocompatibility, further, on the basis of Scheme 4, a functional group B is further introduced through the second functional monomer.

In this developed cross-linking scheme (referred as Scheme 5), a first functional monomer with a first carbon-carbon double bond and a residual amino group is introduced before glutaraldehyde cross-linking. The first functional monomer first physically permeates into the biological material, and then undergoes co-crosslinking with an aldehyde-based cross-linking agent. The amino group of the first functional monomer reacts with the amino group on the biological valve through the aldehyde group of the aldehyde-based cross-linking agent to introduce the first carbon-carbon double bond to the biological material. Further, a functional group B is introduced. Finally, a copolymerization is initiated between the second functional monomer and the carbon-carbon double bonds on the biological material, forming a cross-linked network while introducing the functional group B, thereby further improving the cross-linking degree and performance of the biological material.

Specifically, Scheme 5 includes:
AS110: soaking a biological material in a solution containing a first functional monomer for physical permeation, wherein the first functional monomer has at least one amino group and at least one first carbon-carbon double bond;
AS120: adding an aldehyde-based cross-linking agent to the system in step AS110 to perform co-crosslinking;
AS130: soaking the biological material treated in step AS120 into a solution containing a second functional monomer for physical permeation, wherein the second functional monomer has at least one second carbon-carbon double bond and at least one functional group B; and
S200: adding an initiator to the system in step AS130 to initiate double bond polymerization.

The reaction mechanism in this scheme is the same as that in Scheme 4. The difference between the two is that a functional group B is also introduced while introducing the second carbon-carbon double bond. The functional group B of the second functional monomer can give new characteristics to the biological material.

In this scheme, the biological material is cross-linked through aldehyde co-crosslinking and secondary cross-linking of double bond polymerization. The biological material treated by dual cross-linking has a good degree of cross-linking. During the cross-linking, the first functional monomer can react with part of the residual aldehyde groups. After introducing the first carbon-carbon double bonds through the co-crosslinking, the second carbon-carbon double bonds are further introduced through the permeation of the second functional monomer. The two-step introduction of carbon-carbon double bonds allows a copolymerization to be initiated with the additional functional monomer involved during the double bond polymerization process, forming a larger polymer cross-linked network, which is beneficial to improving the cross-linking degree and anti-calcification performance of the biological valve. While introducing the second carbon-carbon double bond, the functional group B is also introduced, which can give new characteristics to the biological material and further improve the performance of the biological material.

In preferred embodiments, optionally, after step AS120 of Scheme 1 Scheme 2, and Scheme 3, and before step AS130 of Scheme 4 and Scheme 5, step AS120(M) is further included: soaking the biological material treated in step AS120 in a solution containing a third functional monomer to eliminate the remaining residual aldehyde groups, wherein the third functional monomer in this step has at least one group that can react with the aldehyde group.

In Scheme 1, both the first functional monomer and the third functional monomer have at least one group that can react with the aldehyde group. The first functional monomer reacts with the aldehyde group through this group during the co-crosslinking process. In the scheme including step AS120(M), the third functional monomer reacts with the residual aldehyde group on the biological material through this group to eliminate the residual aldehyde group. Optionally, the groups that can react with the aldehyde group in the first functional monomer and the third functional monomer are each independently selected from amino group or hydrazide.

Optionally, in the scheme where the group that can react with the aldehyde group is an amino group, the first functional monomer and the third functional monomer are each independently selected from at least one amino-substituted alkane, at least one amino-substituted cycloalkane, at least one amino-substituted alkene or amino-containing polymer.

In the scheme where the group that can react with the aldehyde group is an amino group, further optionally, the first functional monomer and the third functional monomer are each independently selected from one of ethylenediamine, 2-methylpropylamine, 1,4-butanediamine, n-hexylamine, oleylamine, 1,10-diaminodecane, octylamine, n-undecylamine, dodecylamine, tetradecylamine, hexadecylamine, heptadecane-9-amine, cyclododecylamine, cycloheptylamine, cyclooctylamine, 6-methylheptane-1-amine, nonadecan-10-amine, 3-ethylpentan-1-amine, 2-methylallylamine, 3-butene-1-amine, 4-penten-1-amine, and 2-aminoethyl methacrylate.

In the scheme where the group that can react with the aldehyde group is hydrazide, optionally, the first functional monomer and the third functional monomer are each independently selected from methacryloyl hydrazide or acryloyl hydrazide.

In Scheme 1, in addition to the amino group, the first functional monomer and the third functional monomer can also have a functional group A. Optionally, the functional group A is a hydrophilic group. Further optionally, the functional group A of the first functional monomer and the third functional monomer is each independently selected from at least one of hydroxyl group, carboxyl group, amide group, sulfonic acid group, zwitterion, polyethylene glycol, urea group, carbamate group, carboxylate ion, sulfonate, sulfoxide, and pyrrolidone.

In the functional group A of:
hydroxyl group: as a hydrophilic group, it improves the surface hydrophilicity of the co-cross-linked biological material to achieve anticoagulant effect;
carboxyl group: as a hydrophilic group, it improves the surface hydrophilicity of the co-cross-linked biological material to achieve anticoagulant effect;
amide group: as a hydrophilic group, it improves the surface hydrophilicity of the co-cross-linked biological material to achieve anticoagulant effect;
carboxylate ion and sulfonic acid group: it improves the surface hydrophilicity of the co-cross-linked biological material through ion hydration to achieve anticoagulant effect;
sulfoxide and pyrrolidone: as hydrophilic groups, they improve the surface hydrophilicity of the co-cross-linked biological material to achieve anticoagulant effect;
zwitterion: it improves the surface hydrophilicity of the co-cross-linked biological material through ion hydration to achieve anti-coagulant effect, and it is beneficial to form an electrically neutral surface of the co-cross-linked biological valve, thereby reducing the adsorption of calcium ions to achieve anti-calcification effect;
polyethylene glycol: as a hydrophilic group, it improves the surface hydrophilicity of the co-cross-linked biological material, and increases the steric hindrance between calcium ions and collagen, improving the surface hydrophilicity of the co-cross-linked biological valve material;
carbamate group and urea group: as hydrophilic groups, they improve the surface hydrophilicity of the co-cross-linked biological material to achieve anticoagulant effect; and
carbamate group: as a hydrophilic group, it improves the surface hydrophilicity of the co-cross-linked biological material to achieve anticoagulant effect.

In the scheme with both amino group and hydrophilic functional group, optionally, the first functional monomer and the second functional monomer are each independently selected from one of 2-amino-4-pentanoic acid, 2-amino-octanoic acid, 2-amino-5-hydroxypentanoic acid, 2-amino-2,3-dimethylbutyramide, 2-amino-tetradecanoic acid, 2-amino-4-methylpentanoic acid, trihydroxymethyl aminomethane, amino-terminated polyethylene glycol and polyethylene glycol derivatives, aminooleic acid, natural amino acids, unnatural amino acids, polynatural amino acids (such as polylysine), DL-2-amino-4-pentenoic acid, 2-amino-7-en-octanoic acid, 6-en-aminoheptanoic acid, 2-aminopent-4-enoic acid, 4-(1-amino-2-methyl-propyl)-hepta-1,6-diene-4-ol, 4-(1-amino-ethyl)-hepta-1,6-diene-4-ol, and double-bonded polylysine.

Regarding the first functional monomer and the second functional monomer in Scheme 1, it can be understood that the first functional monomer and the third functional monomer are each independently selected from the above options, and may be the same or different.

The first functional monomer in Scheme 2 has at least one group that can react with an aldehyde group. During the co-crosslinking process, this functional monomer reacts with the residual aldehyde group on the biological material through this group to introduce the carbon-carbon double bond to the biological material. In the scheme including step AS120(M), the third functional monomer has at least one group that can react with an aldehyde group, and this group reacts with the residual aldehyde group on the biological material to eliminate the residual aldehyde group. The group that can react with the aldehyde group in the first functional monomer and the third functional monomer includes but is not limited to amino group and hydrazide. Optionally, the third functional monomer can also have at least one carbon-carbon double bond. When the biological material is treated with the third functional monomer solution, the amino group of the third functional monomer reacts with the residual aldehyde group on the biological material, blocking the remaining residual aldehyde group with carbon-carbon double bonds further introduced, increasing the number of carbon-carbon double bonds for subsequent double bond polymerization, which is beneficial to improving the degree of cross-linking. That is, the groups that can react with the aldehyde group in the first functional monomer and the third functional monomer are each independently selected from one of amino group and hydrazide, and may be the same or different. The functional monomer having at least one amino group and at least one carbon-carbon double bond can directly use commercially available products. Optionally, the first functional monomer and the third functional monomer in the second scheme are each independently selected from at least one of DL-2-amino-4-pentenoic acid, 2-methylallylamine, 3-butene-1-amine, 4-penten-1-amine, 2-aminoethyl methacrylate, methacryloyl hydrazide and acryloyl hydrazide.

The first functional monomer in Scheme 3 has at least one active group. During the co-crosslinking process, this functional monomer reacts with the residual aldehyde group on the biological material through this active group to introduce the first carbon-carbon double bond to the biological material, wherein the active group of the first functional monomer can be amino group or hydrazide.

In the scheme including step AS120(M), the third functional monomer has at least one active group, and the third functional monomer reacts with the residual aldehyde group on the biological material through this active group to eliminate the residual aldehyde group. The active group of the third functional monomer may be amino group or hydrazide. Optionally, the third functional monomer can also have at least one carbon-carbon double bond. When the biological material is treated with the third functional monomer solution, the amino group of the third functional monomer reacts with the residual aldehyde group on the biological material, blocking the remaining residual aldehyde group with carbon-carbon double bonds further introduced, increasing the number of carbon-carbon double bonds for subsequent double bond polymerization, which is beneficial to improving the degree of cross-linking.

In this scheme, in addition to the carbon-carbon double bond and the amino group, the first functional monomer may also have a functional group A, and in addition to the carbon-carbon double bond and the amino group, the third functional monomer may also have a functional group C. Optionally, the functional group A and the functional group C are each independently selected from at least one of hydroxyl group, carboxyl group, amide group and sulfonic acid group. That is, the functional group A of the first functional monomer is at least one of hydroxyl group, carboxyl group, amide group and sulfonic acid group; the functional group C of the third functional monomer is also at least one of hydroxyl group, carboxyl group, amide group and sulfonic acid group; and the may be the same or different.

The introduced hydroxyl group can improve the hydrophilicity of the biological valve; the introduced carboxyl group can maintain the neutral pH of the reaction system in step AS110; the introduced amide group can increase the hydrophilicity of the biological valve through hydrogen bonding interaction between water molecule and amide group; the introduced sulfonic acid group can increase the hydrophilicity of the biological valve through ion hydration between water molecule and sulfonic acid group.

In one scheme, the first functional monomer and the third functional monomer can directly use commercially available products. Optionally, the first functional monomer and the third functional monomer are each independently selected from one of DL-2-amino-4-pentenoic acid, 2-amino-7-en-octanoic acid, 6-en-aminoheptanoic acid, 2-aminopent-4-enoic acid, 4-(1-amino-2-methyl-propyl)-hepta-1,6-diene-4-ol, and 4-(1-amino-ethyl)-hepta-1,6-diene-4-ol.

The first functional monomer in Scheme 4 has at least one group that can react with an aldehyde group. During the co-crosslinking process, the first functional monomer reacts with part of the residual aldehyde groups on the biological material through this group to introduce the first carbon-carbon double bond to the biological material. Optionally, the group of the first functional monomer that can react with the aldehyde group includes but is not limited to amino group and hydrazide. In the scheme including step AS120(M), the third functional monomer has at least one group that can react with an aldehyde group and reacts with the remaining residual aldehyde group on the biological material during soaking. Optionally, the group of the third functional monomer that can react with the aldehyde group includes but is not limited to amino group and hydrazide.

The first functional monomer in this scheme has at least one amino group and at least one first carbon-carbon double bond, which, in one scheme, can directly use commercially available products. Optionally, the first functional monomer is one of 2-methylallylamine, 3-butene-1-amine, 4-penten-1-amine, 2-aminoethyl methacrylate, methacryloyl hydrazide and acryloyl hydrazide.

The third functional monomer in this scheme has at least one amino group. In a more preferred scheme, the third functional monomer also has at least one carbon-carbon double bond. When the biological material is treated with the third functional monomer solution, the amino group of the third functional monomer reacts with the residual aldehyde group on the biological material, blocking the remaining residual aldehyde group with carbon-carbon double bonds further introduced, increasing the number of carbon-carbon double bonds for subsequent double bond polymerization.

Optionally, the third functional monomer is one of 2-methylallylamine, 3-butene-1-amine, 4-penten-1-amine, 2-aminoethyl methacrylate, methacryloyl hydrazide and acryloyl hydrazide.

In this scheme, in addition to the carbon-carbon double bond and the amino group, the first functional monomer may also have a functional group A, and in addition to the carbon-carbon double bond and the amino group, the third functional monomer may also have a functional group C. Optionally, the functional group A and the functional group C are each independently selected from at least one of hydroxyl group, carboxyl group, amide group and sulfonic acid group.

The introduced hydroxyl group can improve the hydrophilicity of the biological valve; the introduced carboxyl group can make the biological material electrically neutral; the introduced hydroxyl group can improve the hydrophilicity of the biological valve; the introduced carboxyl group can maintain the neutral pH of the reaction system in step AS110; the introduced amide group can increase the hydrophilicity of the biological valve through hydrogen bonding interaction between water molecule and amide group; the introduced sulfonic acid group can increase the hydrophilicity of the biological valve through ion hydration between water molecule and sulfonic acid group.

In one scheme, the functional monomer having at least one amino group, at least one carbon-carbon double bond and at least one functional group simultaneously as mentioned above can directly use commercially available products. Optionally, the first functional monomer and the third functional monomer are each independently selected from one of 2-amino-7-en-octanoic acid, 6-en-aminoheptanoic acid, 2-aminopent-4-enoic acid, 4-(1-amino-2-methyl-propyl)-hepta-1,6-diene-4-ol, and 4-(1-amino-ethyl)-hepta-1,6-diene-4-ol.

After step AS120 or step AS120(M) of Scheme 4 is completed, a second carbon-carbon double bond is further introduced through the second functional monomer by physical permeation. The second functional monomer does not react with the biological material in this step. In one scheme, optionally, the second functional monomer is one of polyethylene glycol diacrylate, 1,4-butanediol diacrylate, ethyl acrylate, N-methyl-2-acrylamide, N-2,2-propenyl-2-acrylamide, N-ethylacrylamide, N,N′-ethylenebisacrylamide, and (ethane-1,2-diylbis(oxy))bis(ethane-2,1-diyl)diacrylate.

In Scheme 5, the first functional monomer has at least one amino group and at least one first carbon-carbon double bond, which, in one scheme, can directly use commercially available products. Optionally, the first functional monomer is one of 2-methylallylamine, 3-butene-1-amine, 4-penten-1-amine, 2-aminoethyl methacrylate, methacryloyl hydrazide and acryloyl hydrazide.

The third functional monomer in Scheme 5 has at least one amino group. In a more preferred scheme, the third functional monomer also has at least one carbon-carbon double bond. When the biological material is treated with the third functional monomer solution, the amino group of the functional monomer reacts with the remaining residual aldehyde group on the biological material, blocking the remaining aldehyde group with carbon-carbon double bonds further introduced.

Optionally, the third functional monomer is one of 2-methylallylamine, 3-butene-1-amine, 4-penten-1-amine, 2-aminoethyl methacrylate, methacryloyl hydrazide and acryloyl hydrazide.

In Scheme 5, in addition to carbon-carbon double bond and amino group, the first functional monomer and the third functional monomer can also have functional groups. Optionally, the first functional monomer also has at least one functional group A, and the third functional monomer also has at least one functional group C, wherein the functional group A and functional group B described in this scheme are each independently selected from one of hydroxyl group, carboxyl group, amide group and sulfonic acid group.

The introduced hydroxyl group can improve the hydrophilicity of the biological valve; the introduced carboxyl group can make the biological material electrically neutral; the introduced hydroxyl group can improve the hydrophilicity of the biological valve; the introduced carboxyl group can maintain the neutral pH of the reaction system in step AS110; the introduced amide group can increase the hydrophilicity of the biological valve through hydrogen bonding interaction between water molecule and amide group; the introduced sulfonic acid group can increase the hydrophilicity of the biological valve through ion hydration between water molecule and sulfonic acid group.

In the scheme where the first functional monomer and the third functional monomer have functional groups, they can directly use commercially available products. Optionally, the first functional monomer and the third functional monomer are each independently selected from one of DL-2-amino-4-pentenoic acid, 2-amino-7-en-octanoic acid, 6-en-aminoheptanoic acid, 2-aminopent-4-enoic acid, 4-(1-amino-2-methyl-propyl)-hepta-1,6-diene-4-ol, and 4-(1-amino-ethyl)-hepta-1,6-diene-4-ol.

In Scheme 5, after step AS120 or step AS120(M) is completed, the biological material is in contact with a solution containing a second functional monomer which physically permeates into the biological material. In addition to double bond, the second functional monomer can also have a functional group B which can improve the performance of the biological material. Optionally, the functional group B is one of hydroxyl group, carboxyl group, choline carboxylate, choline sulfonate, choline phosphate, pyrrolidone, sulfonic acid group, carboxylate ion, sulfonate, sulfoxide, amide group, methoxy group.

In one scheme, the second functional monomer is one of polyethylene glycol diacrylate, acrylamide, 2-(prop-2-enoylamino)acetic acid, 2-acrylamido-2-methylpropanesulfonic acid, hydroxyethyl methacrylate, 3-[[2-(methacryloyloxy)ethyl]dimethylammonio]propionate, N-methyl-2-acrylamide, N-isopropylacrylamide, N-(hydroxymethyl)acrylamide, N-(2-hydroxyethyl)methacrylamide, N,N-dimethylmethacrylamide, 3-[N,N-dimethyl-[2-(2-methylprop-2-enoyloxy)ethyl]ammonium]propane-1-sulfonate, 2-methacryloyloxyethyl phosphorylcholine, which is available commercially.

For Schemes 4 and 5, in addition to being commercially available as mentioned above, the second functional monomer can be alternatively available by modification preparation of double bonds. Optionally, the second functional monomer is double-bonded hyaluronic acid or double-bonded polylysine.

For Schemes 1 to 5, in addition to being commercially available as mentioned above, the first functional monomer and the third functional monomer can be alternatively available by modification preparation of double bonds, such as double-bonded polylysine.

That is, the first functional monomer, the second functional monomer and the third functional monomer can be each independently selected from double-bonded hyaluronic acid or double-bonded polylysine.

For Schemes 1 to 5, the first functional monomer and the third functional monomer are each independently selected from the above options (including commercial availability and modification preparation), and may be the same or different.

An implementation of preparing modified double-bonded hyaluronic acid includes:
weighing 2 g of sodium hyaluronate with a molecular weight of 10,000, dissolving it in 20 ml of PBS, and then adding 6-12 ml of glycidyl methacrylate and 4-8 ml of triethylamine in sequence; placing the mixture on a shaker at 37° C. for 5-10 days; finally, using a dialysis bag with a molecular weight cutoff of 5000 to dialyze for 5-7 days, and freeze-drying to obtain double-bonded hyaluronic acid (the preparation can be scaled up in equal proportions as required);

An implementation of preparing double-bonded polylysine includes:
dissolving polylysine in deionized water, then adding glycidyl methacrylate at a molar ratio in a range of 1:1.5 to 1:5 (glycidyl methacrylate:amino group); placing the mixture on a shaker at 37° C. for 5-10 days; finally, using a dialysis bag with a molecular weight cutoff of 1,000 to dialyze for 5-7 days, and freeze-drying to obtain partially double-bonded polylysine.

The biological material in the present disclosure needs to undergo conventional pretreatment before introducing functional monomers. Optionally, the pretreatment includes conventional cleaning operation: obtaining a biological material and storing it in a low-temperature and humid state at 4° C.; cleaning the biological material with distilled water for 2 hours under gentle friction and fluid pressure at 4° C. with shaking at 100 RPM until there were no visible adherent non-pericardial or non-collagenous tissues.

The pretreated biological material is contacted with a solution containing a first functional monomer. Optionally, the contact can be static contact or dynamic contact. In the case where static contact is used, the biological material is just placed and soaked in the solution containing the first functional monomer. In case of dynamic contact, a shaker can be used to shake during the soaking process. During the contact process with the first functional monomer, the temperature can be in the range of 20 to 50° C. Preferably, the temperature in the contact process does not need to be specially controlled. The temperature can be at room temperature. It is suitable not to exceed the human body's adaptive temperature, preferably between 36 and 37° C.

The concentration of the first functional monomer in step AS110 and the contact time between the biological material and the solution containing the first functional monomer are set to ensure that more first functional monomers permeate into the biological material. Generally, the higher concentration of the first functional monomer, the corresponding contact time can be shorter; the lower concentration of the first functional monomer, the corresponding contact time can be longer.

Optionally, the solvent of the solution described in step AS110 is water, physiological saline, neutral pH buffer or an aqueous solution of ethanol. In the aqueous solution of ethanol, ethanol and water can be mixed in any proportion, and commonly used is about 50% ethanol. The concentration of the functional monomer in the solution is in the range of 10 to 100 mM.

Optionally, when the concentration of the first functional monomer is in the range of 10 to 100 mM, the contact time is in the range of 2 to 20 hours, so that the first functional monomer can sufficiently permeate into the biological material.

Further optionally, the concentration of the first functional monomer in the solution described in step AS110 is in the range of 10 to 30 mM, and the soaking time is in the range of 2 to 5 h.

After the first functional monomer has permeated into the biological material, a cross-linking agent is added to the reaction system. Optionally, the concentration of the cross-linking agent is in the range of 10 to 800 mM.

During the co-crosslinking process, the temperature can be between 20° C. and 50° C. Preferably, the temperature does not need to be specially controlled during the co-crosslinking process. The temperature can be at room temperature. It is suitable not to exceed the human body's adaptive temperature. Optionally, the temperature can be in the range of 36 to 37° C. The reaction time for the co-crosslinking should be sufficient to ensure thorough completion of the crosslinking reaction. Optionally, when the concentration of the cross-linking agent is in the range of 10 to 800 mM, the co-crosslinking time is in the range of 10 to 30 hours.

Further optionally, the concentration of the cross-linking agent in step AS120 is in the range of 50 to 500 mM; further, the concentration of the cross-linking agent in step AS120 is in the range of 50 to 150 mM, and the co-crosslinking time is in the range of 20 to 30 h.

Optionally, during co-crosslinking, the contact between the biological material and the solution containing the cross-linking agent can be static contact or dynamic contact. During the dynamic contact process, the reaction system can be shaken while soaking to accelerate the cross-linking process.

The concentration of the third functional monomer and the soaking time in step AS120(M) are set to ensure that more residual aldehyde groups are blocked. Optionally, in step AS120(M), the concentration of the third functional monomer in the solution is in the range of 10 to 100 mM, and the soaking time is in the range of 2 to 48 h.

Further optionally, the solvent in the solution described in step AS120(M) is water, physiological saline, neutral pH buffer or an aqueous solution of ethanol. In the aqueous solution of ethanol, ethanol and water can be mixed in any ratio, and commonly used is 50% ethanol. The concentration of the third functional monomer in the solution containing the third functional monomer is in the range of 10 to 100 mM, and the soaking time is in the range of 2 to 48 h.

Further, the concentration of the third functional monomer in Scheme 1 is in the range of 30 to 50 mM, and the soaking time is in the range of 10 to 20 h.

The concentration of the third functional monomer in Scheme 2 is in the range of 10 to 30 mM, and the soaking time is in the range of 3 to 8 h.

The concentration of the third functional monomer in Scheme 3 is in the range of 30 to 50 mM, and the soaking time is in the range of 3 to 8 hours.

The concentration of the third functional monomer in Scheme 4 is in the range of 20 to 50 mM, and the soaking time is in the range of 3 to 8 hours.

The concentration of the third functional monomer in the solution described in Scheme 5 is in the range of 20 to 40 mM, and the soaking time is in the range of 2 to 4 h. In the step AS120(M), the biological material treated in step AS120 is washed and then soaked in the third functional monomer solution; or the biological material treated in step AS120 is directly transferred to the third functional monomer solution.

In the step AS120(M), the temperature can be in the range of 20 to 50° C. Preferably, the soaking temperature does not need to be specially controlled. The temperature can be at room temperature. It is suitable not to exceed the human body's adaptive temperature, preferably between 36 and 37° C.

In Schemes 4 to 5 in the present disclosure, carbon-carbon double bonds are further introduced (i.e. in step AS130 in Schemes 4 and 5) after co-crosslinking or step AS120(M) is completed, and a secondary cross-linking is completed through double bond polymerization. In an optional scheme, the second functional monomer is introduced directly after the co-crosslinking or step AS120(M) is completed. This scheme is commonly known as the one-pot method, in which, after the co-crosslinking is completed, the second functional monomer is directly added to the reaction system after the co-crosslinking or step AS120(M). After the second functional monomer permeates into the biological material, an initiator is directly added to the reaction system to initiate double bond polymerization, without need to remove the biological material for cleaning.

In an alternative scheme, a step of cleaning the biological material after co-crosslinking or step AS120(M) is completed is included. In this scheme, the biological material is taken out after co-crosslinking or step AS120(M), and cleaned to remove residual functional monomers, cross-linking agents, etc., and then immersed in a solution containing a second functional monomer for contact, with double bond polymerization initiated.

The co-crosslinked biological material is contacted with the solution containing the second functional monomer to further introduce carbon-carbon double bonds. The final concentration of the second functional monomer and the contact time between the biological material and the solution containing the second functional monomer are set to ensure that more second functional monomers permeate into the biological material. Generally, the higher concentration of the second functional monomer, the corresponding contact time can be shorter; the lower concentration of the second functional monomer, the corresponding contact time can be longer.

Optionally, the solvent in the solution containing the second functional monomer is water, physiological saline or neutral pH buffer or an aqueous solution of ethanol. In the aqueous solution of ethanol, ethanol and water can be mixed in any proportion, commonly used is about 50% ethanol. The mass percentage concentration of the second functional monomer is in the range of 1 to 10%.

Optionally, when the mass percentage concentration of the second functional monomer is in the range of 1 to 10%, the contact time is in the range of 2 to 20 h, so that the second functional monomer can sufficiently permeate into the biological material.

Further optionally, the mass percentage concentration of the second functional monomer in the solution containing the second functional monomer is in the range of 2 to 5%, and the soaking time is in the range of 10 to 15 h.

Optionally, the contact process between the biological material and the solution containing the second functional monomer can be static contact or dynamic contact. The temperature during the contact process can be at 20 to 50° C. Preferably, the temperature does not need to be specially controlled and can be at room temperature. It is suitable not to exceed the human body's adaptive temperature, preferably between 36 and 37° C.

In Schemes 2 and 3, after completing the co-crosslinking, first carbon-carbon double bonds are introduced to the biological material, and further, the polymerization of the carbon-carbon double bonds is initiated to perform the secondary cross-linking (i.e., step AS130 in Schemes 2 and 3). In an optional scheme, double bond polymerization is initiated directly after the co-crosslinking is completed. This scheme is commonly known as the one-pot method, in which an initiator is directly added to the reaction system after the co-crosslinking is completed, without the need to remove the biological materials for cleaning. In an alternative scheme, a step of cleaning the biological material after co-crosslinking is included. In this scheme, the biological material is taken out after co-crosslinking, and cleaned to remove residual functional monomers, cross-linking agents, etc., and then soaked in a solution containing an initiator.

In the preferred scheme including step AS120(M), the initiator is directly added to the system after soaking in step AS120(M), or the biological material after soaking in step AS120(M) is washed and then soaked in a solution containing an initiator.

In Schemes 4 and 5, after the second functional monomer permeates into the biological material, an initiator is added to initiate free radical polymerization of the carbon-carbon double bonds and perform secondary cross-linking (i.e., step S200 in Schemes 4 and 5).

In an optional initiating scheme, the initiator is a mixture of ammonium persulfate and sodium bisulfite; the concentrations of ammonium persulfate and sodium bisulfite in the solution are each in the range of 10 to 100 mM; further, the concentrations of ammonium persulfate and sodium bisulfite are each in the range of 20 to 40 mM.

In another optional initiating scheme, the initiator is a mixture of ammonium persulfate and N,N,N′,N′-tetramethylethylenediamine; the mass percentage concentrations of ammonium persulfate and N,N,N′,N′-tetramethylethylenediamine are in the ranges of 2% to 5% and 0.2% to 0.5% respectively.

Optionally, the solvent in the solution containing the initiator is water, physiological saline or neutral pH buffer.

Regarding the concentration of the initiator as mentioned above, in the one-pot method, it can be understood as the concentration of ammonium sulfate and sodium bisulfite in the solution of the reaction system in step AS120, while in the step-by-step method, it can be understood as the concentration of ammonium sulfate and sodium bisulfite in the solution containing the initiator.

Optionally, the double bond polymerization process can be carried out at 20 to 50° C. Preferably, the temperature does not need to be specially controlled during the double bond polymerization process and can be at room temperature. It is suitable not to exceed the human body's adaptive temperature, preferably between 36 and 37° C. The double bond polymerization time is preferably in the range of 2 to 48 hours, preferably in the range of 20 to 25 hours.

Optionally, a post-treatment process after the double bond polymerization is completed is included, and the post-treatment includes conventional cleaning, softening, drying among others.

In order to prepare a wet valve, the biological material can be stored in a solvent after softening treatment. For example, glycerol can be used to store it. In order to prepare a dry valve, the biological material is dried after softening treatment. The drying method is one or more of room temperature drying, blast drying, vacuum drying, and freeze drying. The drying time is in the range of 1 h to 10 days. In case of room temperature drying, the temperature is in the range of 10° C. to 30° C. In case of blast drying or vacuum drying, the temperature is in the range of 15° C. to 100° C. In case of freeze drying, the temperature is in the range of −20° C. to −80° C.

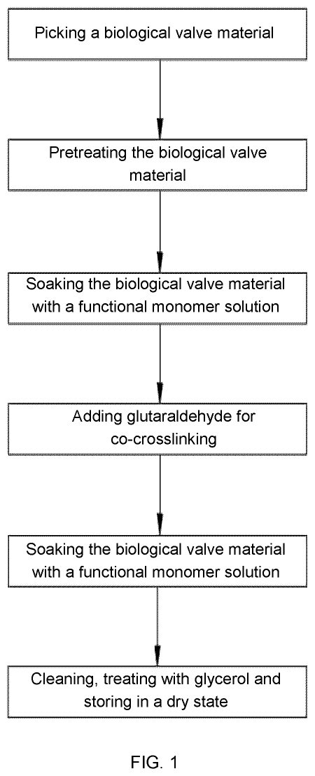

The preferred process shown in FIG. 1 is used as an example to explain Scheme 1 which includes the following steps:
S1: obtaining a biological material and storing it in a low-temperature and humid state at 4° C.;
S2: cleaning the biological material in step S1 with distilled water for 2 hours under gentle friction and fluid pressure at 4° C. with shaking at 100 RPM, until there is no visible adherent non-pericardium or non-collagenous tissue;
S3: then soaking the biological material cleaned in step S2 in an arginine aqueous solution with a molar concentration of 10-100 mM for 12 hours at 37° C. to ensure that the arginine physically permeates into the biological material sufficiently;
S4: adding glutaraldehyde to the solution in which the biological material is soaked and treated in step S3 for co-crosslinking for 24 hours at 37° C., wherein the molar concentration of glutaraldehyde in the solution system is in the range of 10-800 mM;
S5: soaking the biological material treated in step S4 in arginine solution (10-100 mM) again for 2 to 48 hours; and
S6: soaking and cleaning the biological material treated in step S5 with distilled water to remove unreacted arginine and glutaraldehyde.

In the scheme where dry storage is required, the scheme further includes:
S7: performing conventional drying treatment of the biological material.

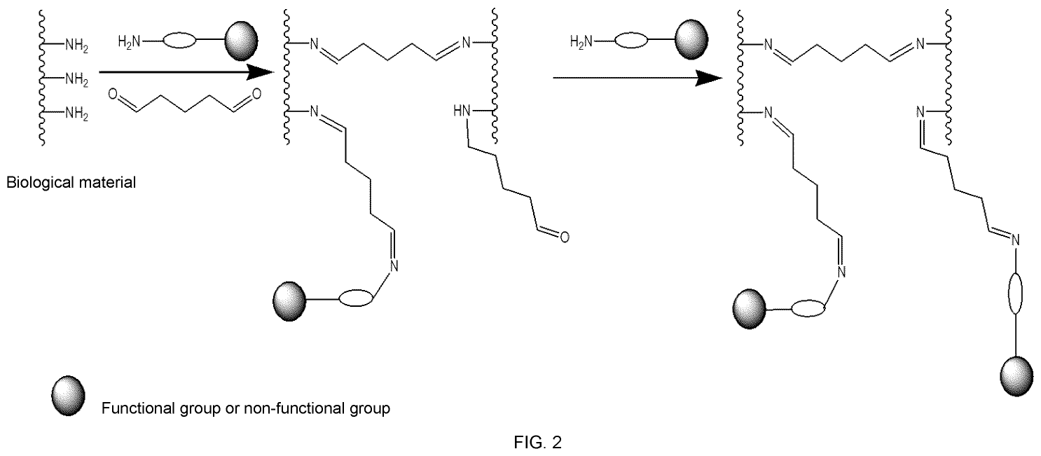

The schematic diagram of the chemical principle of this scheme is shown in FIG. 2.

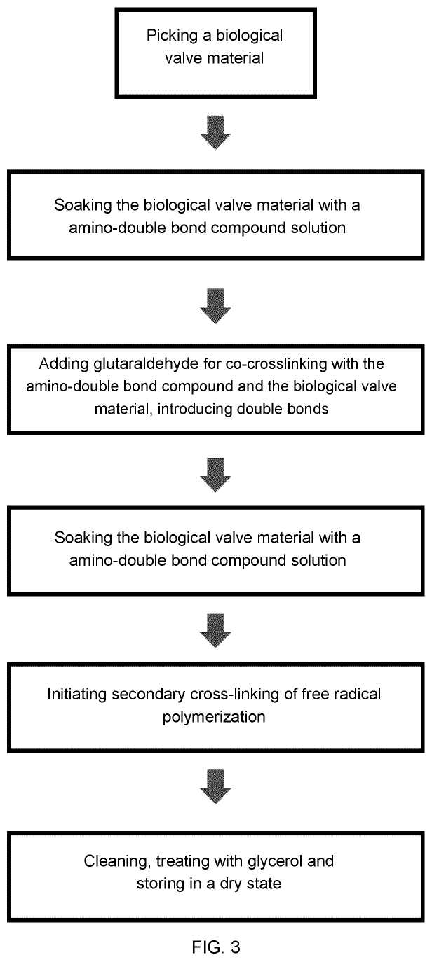

The preferred process shown in FIG. 3 is used as an example to explain Scheme 2 which includes the following steps:
Step 1: picking a biological valve material and performing conventional pretreatment on the biological valve material;
Step 2: soaking the biological material in a first functional monomer (amino-double bond compound) solution;
Step 3: adding a cross-linking agent (glutaraldehyde) to the reaction system in step 2 for co-crosslinking with the first functional monomer (amino-double bond compound) and the biological valve material, introducing free radicals (carbon-carbon double bonds);
Step 4: soaking the biological material treated in step 3 into a third functional monomer solution;
Step 5: initiating secondary cross-linking of free radical polymerization; and
Step 6: after secondary cross-linking, cleaning the biological material and treating it with glycerol, and storing the biological valve in a dry or wet state.

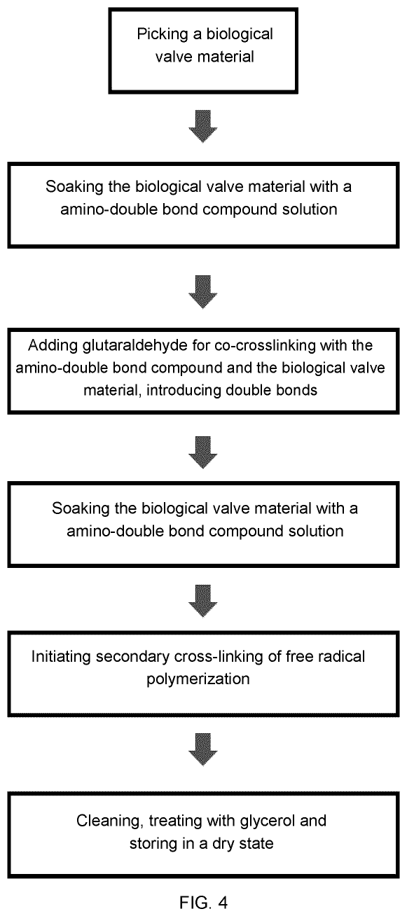

The preferred process shown in FIG. 4 is used as an example to explain Scheme 3 which includes the following steps:
Step 1: picking a biological valve material and performing conventional pretreatment on the biological valve material;
Step 2: soaking the biological valve material in an amino-double bond compound (i.e. functional monomer with functional group) solution;
Step 3: adding glutaraldehyde (cross-linking agent) to the reaction system in step 2 for co-crosslinking with the amino-double bond compound (functional monomer) and the biological valve material, introducing carbon-carbon double bonds (free radicals) and functional groups;
Step 4: soaking the biological material treated in Step 3 in an amino-double bond compound (functional monomer) solution;
Step 5: initiating secondary cross-linking of free radical polymerization; and
Step 6: after secondary cross-linking, cleaning the biological material and treating it with glycerol, and storing the biological valve in a dry or wet state.

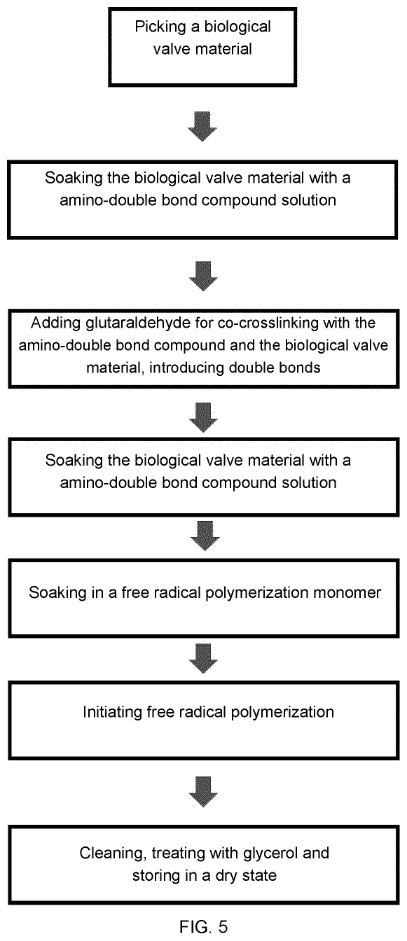

The preferred process shown in FIG. 5 is used as an example to explain Scheme 4 which includes the following steps:
Step 1: picking a biological valve material and performing conventional pretreatment on the biological valve material;
Step 2: soaking the biological material in a first functional monomer (amino-double bond compound) solution;
Step 3: adding a cross-linking agent (glutaraldehyde) to the reaction system in step 2 for co-crosslinking with the functional monomer (amino-double bond compound) and the biological valve material, introducing free radicals (carbon-carbon double bonds), and optionally introducing functional groups;
Step 4: soaking the biological material treated in step 3 in an amino-double bond compound (third functional monomer) solution;
Step 5: soaking in a free radical polymerization monomer (second functional monomer);
Step 6: initiating secondary cross-linking of free radical polymerization; and
Step 7: after secondary cross-linking, cleaning the biological material and treating it with glycerol, and storing the biological valve in a dry or wet state.

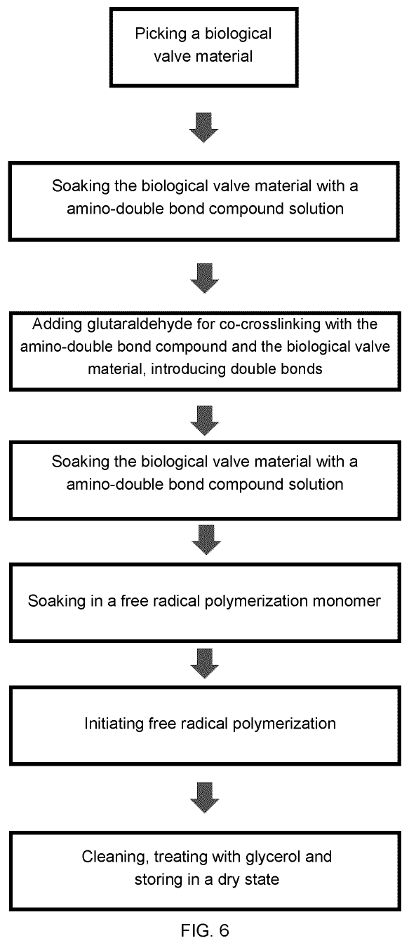

The preferred process shown in FIG. 6 is used as an example to explain Scheme 5 which includes the following steps:
Step 1: picking a biological valve material and performing conventional pretreatment on the biological valve material;
Step 2: soaking the biological material in an amino-double bond compound (first functional monomer) solution;
Step 3: adding glutaraldehyde (cross-linking agent) to the reaction system in step 2 for co-crosslinking with the amino-double bond compound (first functional monomer) and the biological valve material, introducing carbon-carbon double bonds (free radicals)), and optionally introducing functional groups;
Step 4: soaking the biological material treated in step 3 in an amino-double bond compound (third functional monomer) solution;
Step 5: soaking in a free radical polymerization monomer (second functional monomer);
Step 6: initiating secondary cross-linking of free radical polymerization; and
Step 7: after secondary cross-linking, cleaning the biological material and treating it with glycerol, and storing the biological valve in a dry or wet state.

A more specific implementation includes the following steps:
S1: obtaining a biological material and storing it in a low-temperature and humid state at 4° C.;
S2: cleaning the biological material in step S1 with distilled water for 2 hours under gentle friction and fluid pressure at 4° C. with shaking at 100 RPM, until there is no visible adherent non-pericardium or non-collagenous tissue;
S3: then soaking the biological material cleaned in step S2 in a DL-2-amino-4-pentenoic acid aqueous solution with a molar concentration of 10-100 mM for 12 hours at 37° C. to ensure that DL-2-amino-4-pentenoic acid physically permeates into the biological material sufficiently;
S4: adding glutaraldehyde to the solution in which the biological material is soaked and treated in step S3 for copolymerization for 12 hours at 37° C., wherein the molar concentration of glutaraldehyde in the solution system is in the range of 10-500 mM;
S5: soaking and cleaning the biological material treated in step S4 with distilled water to remove unreacted DL-2-amino-4-pentenoic acid and glutaraldehyde;
S6: soaking the biological material treated in step S5 in 5% polyethylene glycol diacrylate aqueous solution at 37° C. for 12 hours to ensure sufficient physical permeation of polyethylene glycol diacrylate; and
S7: adding an initiator of ammonium persulfate and sodium bisulfite to the biological material treated in step S6 for initiation, wherein the molar concentration of ammonium persulfate and sodium bisulfite is in the range of 10-100 mM.

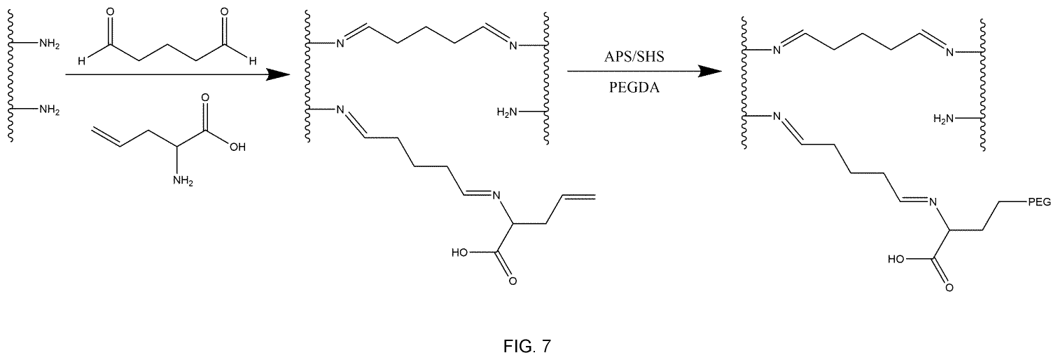

The schematic diagram of the chemical principle of this implementation is shown in FIG. 7.

In this implementation, in step S3, the method of introducing free radical polymerizable allyl groups through co-crosslinking of DL-2-amino-4-pentenoic acid, glutaraldehyde, and pericardium has higher efficiency in introducing free radical polymerizable groups compared with the similar studies reported in the literature, and can further improve the cross-linking degree of the pericardium while introducing allyl groups.

This implementation prepares a biological valve material with excellent anti-calcification and anti-coagulation properties, meeting the performance indicators of a lactate dehydrogenase relative activity in the range of 0.1 to 0.25 and a calcium content in the range of 30 to 50 μg/mg. The copolymerization and cross-linking method used here involves using DL-2-amino-4-pentenoic acid and glutaraldehyde as copolymerization and cross-linking agents to introduce carbon-carbon double bonds to the biological material. Subsequently, polyethylene glycol diacrylate is added to covalently bond polyethylene glycol to the surface of the pericardium through copolymerization and cross-linking under the initiator of ammonium persulfate and sodium bisulfite. This process improves the structural stability, anti-calcification and anti-coagulation properties of the biological valve material, potentially extending its lifespan.

Another more specific implementation includes the following steps:
S1: obtaining a biological material and storing it in a low-temperature and humid state at 4° C.;
S2: cleaning the biological material in step S1 with distilled water for 2 hours under gentle friction and fluid pressure at 4° C. with shaking at 100 RPM, until there is no visible adherent non-pericardium or non-collagenous tissue, and performing effective decellularization of the pericardial tissue through osmotic shock;
S3: weighing 2 g of sodium hyaluronate with a molecular weight of 10,000, dissolving it in 20 ml of PBS, and then adding 6-12 ml of glycidyl methacrylate and 4-8 ml of triethylamine in sequence; placing the mixture on a shaker at 37° C. for 5-10 days; finally, using a dialysis bag with a molecular weight cutoff of 5000 to dialyze for 5-7 days, and freeze-drying to obtain double-bonded hyaluronic acid (the preparation can be scaled up in equal proportions as required);
S4: dissolving polylysine in deionized water, then adding glycidyl methacrylate at a molar ratio in a range of 1:1.5 to 1:5 (glycidyl methacrylate:amino group); placing the mixture on a shaker at 37° C. for 5-10 days; finally, using a dialysis bag with a molecular weight cutoff of 1,000 to dialyze for 5-7 days, and freeze-drying to obtain partially double-bonded polylysine;
S5: soaking the pericardium in S2 in the partially double-bonded polylysine (molar concentration: 100 mM to 500 mM) aqueous solution prepared in S4 for 1-3 days to ensure near-saturated physical permeation, thereby introducing as much partially double-bonded polylysine as possible, and then adding glutaraldehyde to the aqueous solution to a mass concentration of 2.5%;
S6: initiating free radical copolymerization of the biological material treated in step S5 with the double-bonded hyaluronic acid prepared in S3 under the initiation of ammonium persulfate and N,N,N′,N′-tetramethylethylenediamine at 37° C. for 12-24 hours, wherein the concentration of double-bonded hyaluronic acid used is in the range of 20 mg/ml to 60 mg/ml; and
S7: finally, soaking and cleaning the biological material with distilled water to remove the ungrafted double-bonded hyaluronic acid.

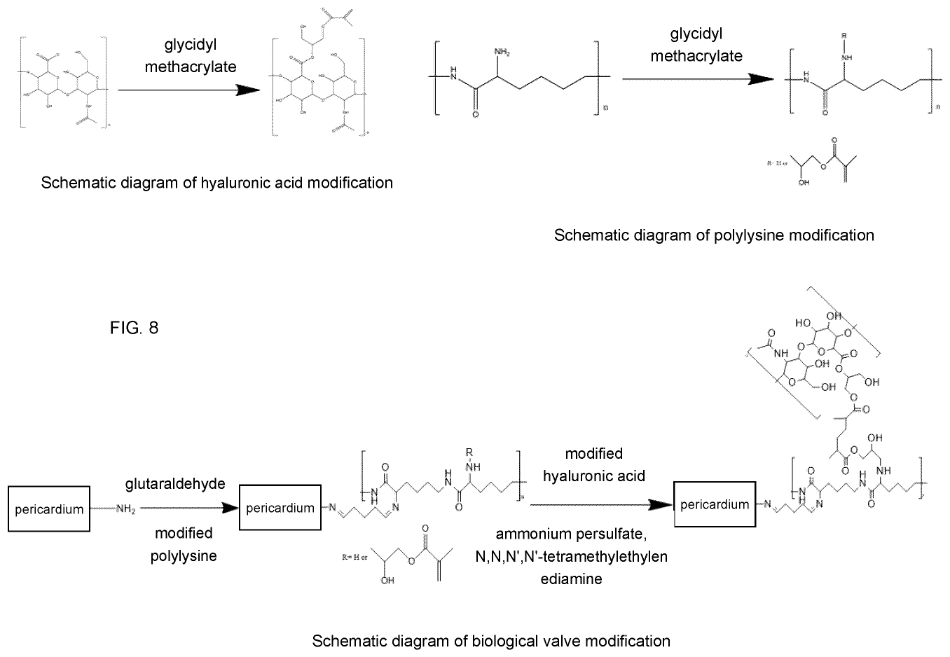

In this implementation, the schematic diagram of the modification of hyaluronic acid, the schematic diagram of the modification of polylysine and the schematic diagram of the modification of pericardium by partially double-bonded polylysine and the principle of free radical polymerization of double-bonded hyaluronic acid are shown in FIG. 8.

Compared with similar studies on hydrophilic treatment of pericardium with polysaccharide that have been reported, this preferred implementation has advantages of:
1) during co-crosslinking of methacrylated polylysine, glutaraldehyde, and pericardium, free radical polymerizable methacrylic groups are introduced, which has a higher efficiency in introducing double bonds, compared with other reported methods (for example, firstly reacting the pericardium with glutaraldehyde and then using residues to introduce double bonds);
2) this method uses double cross-linking, including glutaraldehyde cross-linking and free radical polymerization cross-linking, so the material has a high degree of cross-linking;
3) compared with most studies using polysaccharides for hydrophilic modification of the surface, the binding method between hyaluronic acid and pericardium material in this method is chemical covalent binding, which has higher stability.

To sum up, in this scheme, glycidyl methacrylate is used to modify polylysine and hyaluronic acid to obtain partially double-bonded polylysine and double-bonded hyaluronic acid. Then, under the action of glutaraldehyde, the pericardium and the partially double-bonded polylysine (having both amino groups and double bonds) are copolymerized and cross-linked to simultaneously achieve cross-linking and double bonding modification of the pericardium. Finally, a hyaluronic acid-modified glutaraldehyde pericardium material is obtained by free radical copolymerization of the double-bonded glutaraldehyde valve and the double-bonded hyaluronic acid. This scheme improves the blood compatibility and anti-calcification performance of the biological material, and obtains a biological valve material with a water contact angle of 42.66 to 60.44°, a lactate dehydrogenase activity of 0.22 to 0.26, a hemolysis rate of 0.26 to 0.50% and a calcium ion concentration of 8.28 to 65.62 μg/mg, potentially extending its lifespan.

Almost all existing biological valve products on the market are prepared by cross-linking with glutaraldehyde. Glutaraldehyde can improve the mechanical properties of the pericardium and reduce its immunogenicity to a certain extent, but glutaraldehyde cross-linked biological valves still have problems with low stability and cross-linking degree. This can lead to the degradation of their components after implantation, causing structural damage and consequently structural degradation. Furthermore, the degradation of biological valve components will further induce mechanical damage and calcification, affecting the normal function of the valve and reducing its service life. Glutaraldehyde cross-linking is still the mainstream method for current biological valve products. Therefore, on the basis of glutaraldehyde cross-linking, biological valves are further cross-linked and modified to improve their cross-linking degree and stability. This is of great significance for the development of scientific research and related industries.

The present disclosure initiates post-crosslinking on the basis of glutaraldehyde cross-linking by further introducing double bonds, that is, on the basis of glutaraldehyde cross-linking valve, by introducing first carbon-carbon double bonds through chemical reaction between a first functional monomer (containing the first carbon-carbon double bond and oxirane group) and glutaraldehyde cross-linked biological valve. This scheme is referred as Scheme 6, which will improve the cross-linking degree, stability, mechanical properties and anti-calcification of glutaraldehyde cross-linked biological valve material.

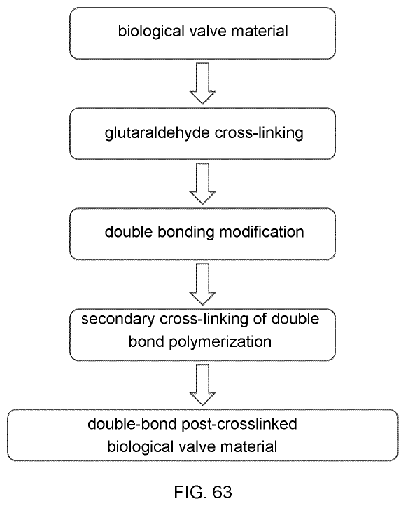

Specifically, Scheme 6 includes (see FIG. 63):
BS110: soaking a biological valve material in a solution containing an aldehyde-based cross-linking agent for cross-linking to prepare glutaraldehyde cross-linked biological valve material;
BS120: soaking the glutaraldehyde cross-linked biological valve material prepared in step BS110 in a solution containing a double bonding reagent (first functional monomer) for double bonding modification to prepare a double bonded biological valve material, wherein the double bonding reagent (first functional monomer) has at least one first carbon-carbon double bond and an oxirane group; and
S200: contacting the biological valve material treated in step BS120 with an initiator to initiate double bond polymerization.

The reaction mechanism of this scheme is explained as follows.

In this double-bond cross-linking scheme, after the biological valve material is cross-linked with glutaraldehyde, the solution containing the first functional monomer (the double bonding reagent) is further used to introduce the first carbon-carbon double bonds for double bonding of the glutaraldehyde cross-linked biological valve material, wherein the first functional monomer (i.e., the double bonding reagent) has both the first carbon-carbon double bond and the oxirane group.

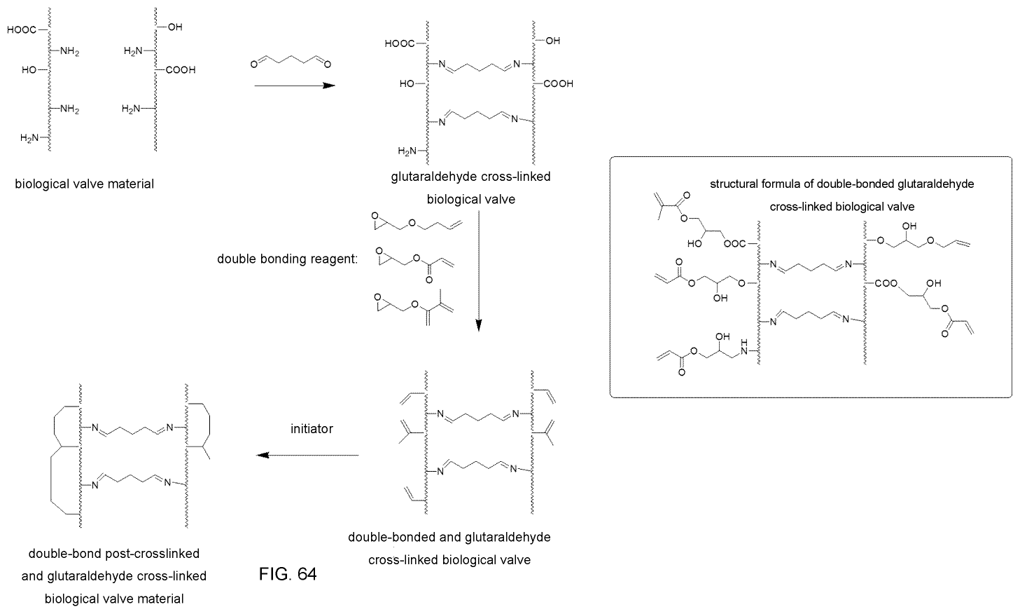

For ease understanding of the chemical mechanism involved in this scheme, FIG. 64 is illustrated as an example for explanation. The first functional monomer (i.e., the double bonding reagent) is used to modify the glutaraldehyde cross-linked biological valve material. A ring-opening reaction is occurred between the oxirane group of the first functional monomer (i.e., the double bonding reagent) and the hydroxyl groups, carboxyl groups as well as the remaining small amount of amino groups after glutaraldehyde cross-linking on the glutaraldehyde cross-linked biological valve material, thereby directly introducing first carbon-carbon double bonds to the glutaraldehyde cross-linked biological valve material. Further, a polymerization of these double bonds on the glutaraldehyde cross-linked biological valve material is initiated to achieve secondary cross-linking and complete the post-crosslinking treatment of the biological valve material. The cross-linking degree of the biological valve material after secondary cross-linking will be further improved, and its stability, mechanical properties and anti-calcification properties will also be further improved.

In step BS120 of this scheme:
optionally, the double bonding reagent (i.e., the first functional monomer) is selected from at least one of allyl glycidyl ether, glycidyl methacrylate, and glycidyl acrylate;
optionally, the concentration of the double bonding reagent in the solution containing the first functional monomer (i.e., the double bonding reagent) is in the range of 1% to 10% (w/w), and the reaction time for the double bonding modification is in the range of 2 to 120 hours;
optionally, the solvent in the solution containing the first functional monomer (i.e., the double bonding reagent) is one or more of water, physiological saline, neutral pH buffer or an aqueous solution of methanol, ethanol, ethylene glycol, propanol, 1,2-propanediol, 1,3-propanediol, isopropanol, butanol, isobutanol, 1,2-butanediol, 1,3-butanediol, 1,4-butanediol, or glycerol;
optionally, the biological valve material treated in S110 is taken out and cleaned or directly placed in the solution containing the double bonding reagent (first functional monomer).

In step S200 of this scheme:
the biological valve material treated in step BS120 is cleaned with deionized water and then immersed in the initiator solution for the treatment in step S200, or the initiator is directly added to the reaction system in step BS120 to initiate the polymerization reaction; wherein the latter is commonly known as the one-pot method;
optionally, the solvent in the solution containing the initiator is water, physiological saline or neutral pH buffer;
the concentration of the initiator as mentioned above can be understood as the concentration of the initiator in the solution of the reaction system in step BS120 in the one-pot method. In the step-by-step method, this concentration can be understood as the concentration of the initiator in the solution containing the initiator;
optionally, the initiator is a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and tetramethylethylenediamine, or a mixture of sodium persulfate and tetramethylethylenediamine; wherein the concentration of each component in the mixture is in the range of 1 to 100 mM;
optionally, the double bond polymerization time is preferably in the range of 3 to 24 hours.

Compared with the prior art, this scheme has at least one of the following benefits:
(1) based on the glutaraldehyde cross-linked biological valve material, this scheme introduces double bonds to the glutaraldehyde cross-linked biological valve material through double bonding modification as the basis for secondary cross-linking; the polymerization of double bonds on the glutaraldehyde cross-linked biological valve material is further initiated to achieve secondary cross-linking, which can further increase the cross-linking degree of the biological valve material, thereby improving the stability of the biological valve material;
(2) this scheme introduces double bonds to the glutaraldehyde cross-linked biological valve material and further initiates the polymerization of the double bonds, thereby improving the stability of the glutaraldehyde cross-linked material and further reducing calcification risk caused by structural degradation, so the glutaraldehyde cross-linked material also has certain anti-calcification properties.

Almost all existing biological valve products used in clinical implantation are made of glutaraldehyde cross-linked biological valve materials. Glutaraldehyde reacts with collagen in the biological valve material, cross-linking the collagen matrix and reducing the immunogenicity of the material while enhancing its mechanical strength. However, the biological valve materials often exhibit low cross-linking degrees after glutaraldehyde cross-linking, making them susceptible to structural degradation. This degradation leads to component breakdown and compromises structural integrity, ultimately resulting in structural deterioration and decay. Additionally, degradation of biological valve components can cause mechanical damage to valve leaflet structures and promote calcification, impeding the normal opening and closing movement of the valve and reducing the lifespan of the biological valve.

Currently, glutaraldehyde cross-linked biological heart valves remain the predominant choice in clinical practice. However, these glutaraldehyde cross-linked biological heart valves still have problems with low stability and cross-linking degree, as well as the risk of structural degradation and failure caused by structural degradation and damage. Therefore, a series of post-crosslinking and modification based on glutaraldehyde cross-linking not only meet the practical production needs, but also holds significant scientific research value.

Therefore, based on the glutaraldehyde cross-linked biological valve material, the present disclosure further introduces carbon-carbon double bonds to the glutaraldehyde cross-linked biological valve material through double bonding treatment of the glutaraldehyde cross-linked biological valve material as the basis for secondary cross-linking. By initiating a copolymerization reaction between the double bonds of the double-bonded and glutaraldehyde cross-linked biological valve material and the double bonds of the functional monomer, a polymer network of the functional monomer is introduced to the glutaraldehyde cross-linked biological valve material, thereby further expanding the cross-linked network. In other words, on the basis of Scheme 6, second carbon-carbon double bonds are further introduced through physical permeation of a second functional monomer (having the second carbon-carbon double bond), which is referred as Scheme 7. This will increase the cross-linking degree of the glutaraldehyde cross-linked biological valve material, improve its structural stability, and further reduce the calcification degree of the material to improve its anti-calcification performance.

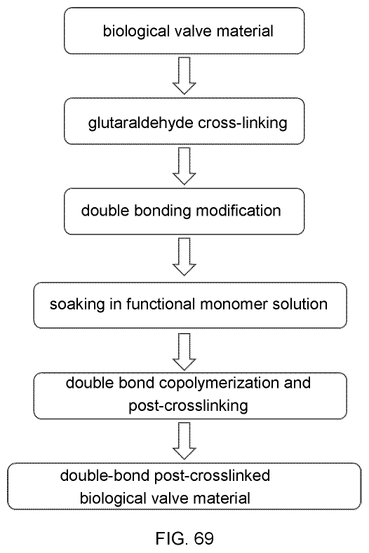

Specifically, Scheme 7 includes (see FIG. 69):
BS110: soaking a biological valve material in a solution containing an aldehyde-based cross-linking agent for cross-linking to prepare glutaraldehyde cross-linked biological valve material;
BS120: soaking the glutaraldehyde cross-linked biological valve material prepared in step BS110 in a solution containing a double bonding reagent (first functional monomer) for double bonding modification to prepare a double bonded biological valve material, wherein the double bonding reagent (first functional monomer) has at least one first carbon-carbon double bond and an oxirane group;
BS130: soaking the double-bonded biological valve material obtained in step BS120 in a solution containing a functional monomer (second functional monomer), wherein the second functional monomer has at least one second carbon-carbon double bond; and
S200: adding an initiator to the solution after soaking in step BS130 to bring it into contact with the biological valve material and the solution containing the functional monomer to initiate double bond polymerization.

The reaction mechanism of this scheme is explained as follows.

In this double-bond cross-linking scheme, after the biological valve material is cross-linked with glutaraldehyde, the solution containing the double bonding reagent (the first functional monomer) is further used to introduce the first carbon-carbon double bonds for double bonding of the glutaraldehyde cross-linked biological valve material as the basis for secondary cross-linking, wherein the double bonding reagent (i.e., the first functional monomer) has both the carbon-carbon double bond and the oxirane group.

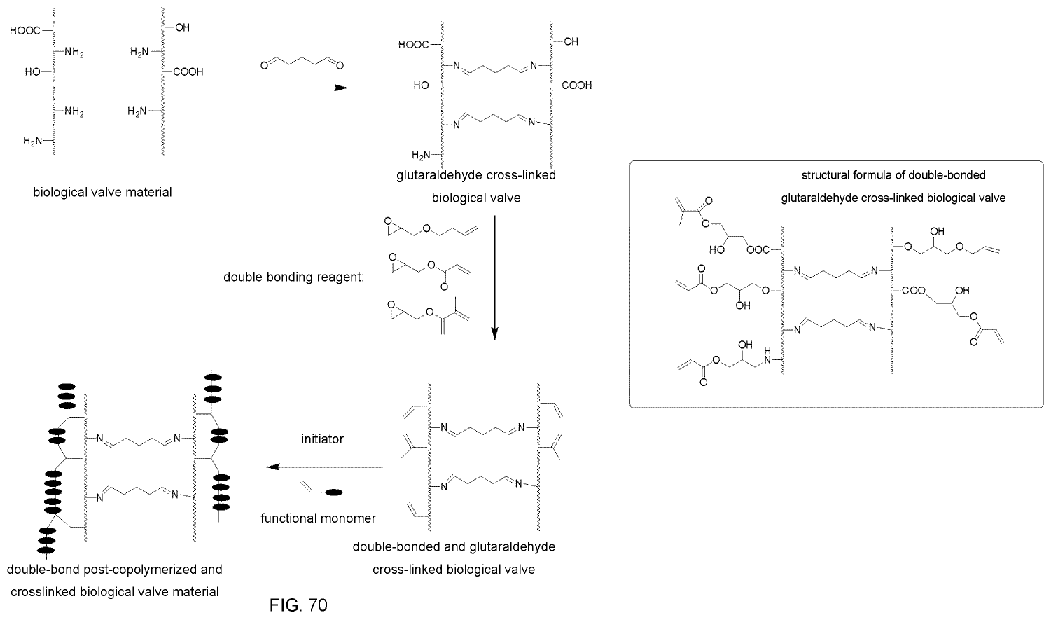

For ease understanding of the chemical mechanism involved in this scheme, FIG. 70 is illustrated as an example for explanation. The double bonding reagent (first functional monomer) is used to modify the glutaraldehyde cross-linked biological valve material. A ring-opening reaction is occurred between the oxirane group of the the double bonding reagent (i.e., the first functional monomer) and the hydroxyl groups, carboxyl groups as well as the remaining small amount of amino groups after glutaraldehyde cross-linking on the glutaraldehyde cross-linked biological valve material, thereby introducing first carbon-carbon double bonds to the glutaraldehyde cross-linked biological valve material. Further, second carbon-carbon double bonds are further introduced through physical permeation of a second functional monomer. Furthermore, a copolymerization between the double bonds of the double-bonded and glutaraldehyde cross-linked biological valve material and the double bonds of the functional monomer is initiated to introduce the polymer of functional monomer as the cross-linked network and to achieve secondary cross-linking, thereby completing the double bond copolymerization and post-crosslinking treatment of the biological valve material.

Since more functional groups (hydroxyl groups and carboxyl groups other than amino groups) on the biological valve material are used for cross-linking, and the polymer of functional monomer is further introduced as a cross-linked network through copolymerization with the functional monomer, the biological valve material has a larger cross-linked network, the degree of cross-linking of the biological valve material after double bond copolymerization and post-crosslinking treatment is significantly improved, and its structural stability and anti-calcification performance are also significantly improved with the introduction of the polymer network of functional monomer.

In step BS120 of this scheme, the double bonding reagent (first functional monomer) selection, the concentration of the double bonding reagent (first functional monomer) solution, the solvent selection of the double bonding reagent (first functional monomer) solution, the reaction time and operation process for double bonding modification are the same as those in Scheme 6, which will not be repeated here.

In step BS130 of this scheme:
the biological valve material treated in step BS120 is soaked in the solution containing the second functional monomer directly or after cleaning;
optionally, the second functional monomer has at least one second carbon-carbon double bond;
further optionally, the second functional monomer is one or more of polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-ethylenebisacrylamide, (ethane-1,2-diylbis(oxy))bis(ethane-2,1-diyl)diacrylate, N,N′-dimethylacrylamide, N,N-dimethylmethacrylamide, and double bonded polylysine;
optionally, the concentration of the solution containing the second functional monomer is in the range of 0.1% to 20% (v/v); further, the concentration of the solution containing the second functional monomer is in the range of 0.1% to 6% (v/v);
optionally, the solvent of the solution containing the second functional monomer is one or more of water, physiological saline, ethanol, isopropanol or neutral pH buffer solution.
optionally, the soaking time in the solution containing the second functional monomer is in the range of 0.5 h to 120 h.

In step S200 of this scheme:
the biological valve material treated in step BS120 is cleaned with deionized water and then immersed in the initiator solution for the treatment in step S200, or the initiator is directly added to the reaction system in step BS120 to initiate the polymerization reaction; wherein the latter is commonly known as the one-pot method;
optionally, the solvent in the solution containing the initiator is water, physiological saline or neutral pH buffer;
the concentration of the initiator as mentioned above can be understood as the concentration of the initiator in the solution of the reaction system in step BS120 in the one-pot method. In the step-by-step method, this concentration can be understood as the concentration of the initiator in the solution containing the initiator;
optionally, the initiator is a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and tetramethylethylenediamine, or a mixture of sodium persulfate and tetramethylethylenediamine; wherein the concentration of each component in the mixture is in the range of 1 to 100 mM;
optionally, the double bond polymerization time is preferably in the range of 3 to 24 hours.

Compared with the prior art, this scheme has at least one of the following benefits:
(1) based on the glutaraldehyde cross-linked biological valve material, this scheme introduces carbon-carbon double bonds to the glutaraldehyde cross-linked biological valve material through double bonding modification as the basis for secondary cross-linking; the polymerization between the carbon-carbon double bonds on the glutaraldehyde cross-linked biological valve material and the carbon-carbon double bonds of the functional monomer is further initiated to introduce a polymer cross-linked network of functional monomer and achieve secondary cross-linking, which can further increase the cross-linking degree of the biological valve material;
(2) this scheme introduces carbon-carbon double bonds to the glutaraldehyde cross-linked biological valve material, and further initiates the polymerization between the carbon-carbon double bonds on the double-bonded biological valve material and the carbon-carbon double bonds of the functional monomer to introduce a polymer cross-linked network of functional monomer, which can further reduce the binding of collagenase in the human body to the collagen matrix on the biological valve material through physical blocking to a certain extent, protecting the collagen matrix of the biological valve material and improving the stability of the glutaraldehyde cross-linked biological valve material, and further reducing calcification risk caused by structural degradation of the biological valve material, so the biological valve material also has certain anti-calcification properties;
(3) this scheme introduces carbon-carbon double bonds to the glutaraldehyde cross-linked biological valve material, and further initiates the polymerization between the carbon-carbon double bonds on the glutaraldehyde cross-linked biological valve material and the carbon-carbon double bonds of the functional monomer to introduce a polymer cross-linked network of functional monomer, which can serve as a polymer barrier to further reduce the binding of calcium ions in the environment to the mineralized areas on the biological valve material that are prone to binding with calcium ions, reducing the risk of calcification and thus playing an anti-calcification role;
(4) this scheme introduces carbon-carbon double bonds to the glutaraldehyde cross-linked biological valve material, and further initiates the polymerization between the carbon-carbon double bonds on the double-bonded and glutaraldehyde cross-linked biological valve material and the carbon-carbon double bonds of the functional monomer to introduce a polymer cross-linked network of functional monomer, thereby increasing the cross-linking degree of the biological valve material and thus making the structure of the biological valve material more rigid while increasing the elasticity; moreover, the polymer cross-linked network of functional monomer fills the gaps between the collagen matrices on the biological valve material to inhibit the deformation of collagen fibers, making the biological valve material harder while increasing the elasticity.

Almost all existing biological valve products used clinically are made of glutaraldehyde cross-linked biological valve materials. Glutaraldehyde reacts with collagen in the biological valve material, cross-linking the collagen matrix and reducing the immunogenicity of the material while enhancing its mechanical strength. However, the biological valve materials often exhibit low cross-linking degrees after glutaraldehyde cross-linking, making them susceptible to structural degradation. This degradation leads to component breakdown and compromises structural integrity, ultimately resulting in structural deterioration and decay. Additionally, degradation of biological valve components can cause mechanical damage to valve leaflet structures and promote calcification, impeding the normal opening and closing movement of the valve and reducing the lifespan of the biological valve. Although it has lower thrombogenicity than mechanical valves, the biological valve still involves thrombosis, which will destroy the normal function of the biological valve and result in a risk of secondary valve replacement. Moreover, the occurrence of calcification will directly lead to the failure of the biological valve.

Therefore, there is still demand for improving the cross-linking degree, stability, anti-thrombotic and anti-calcification properties of the biological valve. Currently, biological valves prepared by cross-linking with glutaraldehyde are still the most commonly used biological valves in clinical practice. In view of the fact that glutaraldehyde cross-linked biological heart valves still have problems such as low stability and cross-linking degree, thrombosis and calcification, as well as the risk of structural degradation and failure caused by these problems, performing a series of functional post-crosslinking on glutaraldehyde cross-linked biological heart valves not only meets the practical production needs but also holds significant scientific research value.

Based on the glutaraldehyde cross-linked biological valve material, the present disclosure further introduces carbon-carbon double bonds to the glutaraldehyde cross-linked biological valve material through double bonding treatment of the glutaraldehyde cross-linked biological valve material as the basis for functionalized copolymerization and cross-linking. By initiating a copolymerization reaction between the double bonds of the double-bonded and glutaraldehyde cross-linked biological valve material and the double bonds of the functional monomer, a functionalized polymer network is introduced to the glutaraldehyde cross-linked biological valve material, thereby further expanding the cross-linked network and achieving post-functionalized copolymerization and cross-linking of double bonds of the biological valve material. In other words, on the basis of Scheme 7, the second functional monomer also has a functional group B, which is referred as Scheme 8. This will increase the cross-linking degree of the glutaraldehyde cross-linked biological valve material and improve its structural stability. The introduced functionalized polymer cross-linked network functionalizes the biological valve material, which will further improve its anti-calcification and anti-thrombotic properties as well as biocompatibility. The introduced functionalized polymer cross-linked network increases the cross-linking degree of the biological valve material and fills the gaps between the collagen matrices to inhibit the deformation of collagen fibers, further making the biological valve material harder while increasing the elasticity.

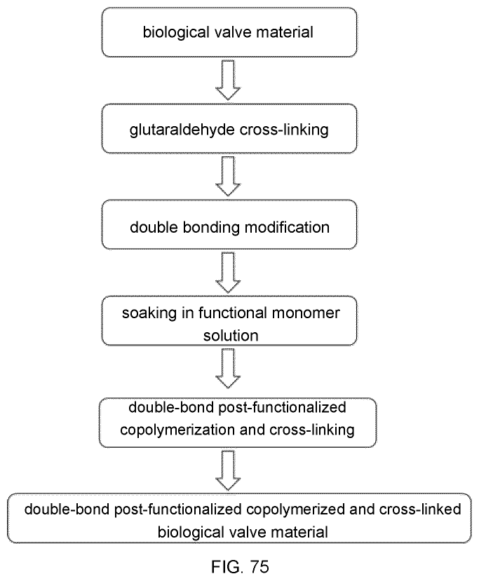

Specifically, Scheme 8 includes (see FIG. 75):
BS110: soaking a biological valve material in a solution containing an aldehyde-based cross-linking agent for cross-linking to prepare glutaraldehyde cross-linked biological valve material;
BS120: soaking the glutaraldehyde cross-linked biological valve material prepared in step BS110 in a solution containing a double bonding reagent (first functional monomer) for double bonding modification to prepare a double bonded biological valve material, wherein the double bonding reagent (first functional monomer) has at least one first carbon-carbon double bond and an oxirane group;
BS130: soaking the double-bonded biological valve material obtained in step BS120 with a solution containing a second functional monomer, wherein the second functional monomer has at least one second carbon-carbon double bond and at least one functional group B; and
S200: adding an initiator to the solution after soaking in step BS130 to bring it into contact with the biological valve material and the solution containing the functional monomer to initiate double bond polymerization.

The reaction mechanism of this scheme is explained as follows.

In this double-bond cross-linking scheme, after the biological valve material is cross-linked with glutaraldehyde, the solution containing the double bonding reagent (the first functional monomer) is further used to introduce the first carbon-carbon double bonds for double bonding of the glutaraldehyde cross-linked biological valve material as the basis for secondary cross-linking, wherein the double bonding reagent (i.e., the first functional monomer) has both the carbon-carbon double bond and the oxirane group, wherein the second functional monomer has a second carbon-carbon double bond and a functional group B.

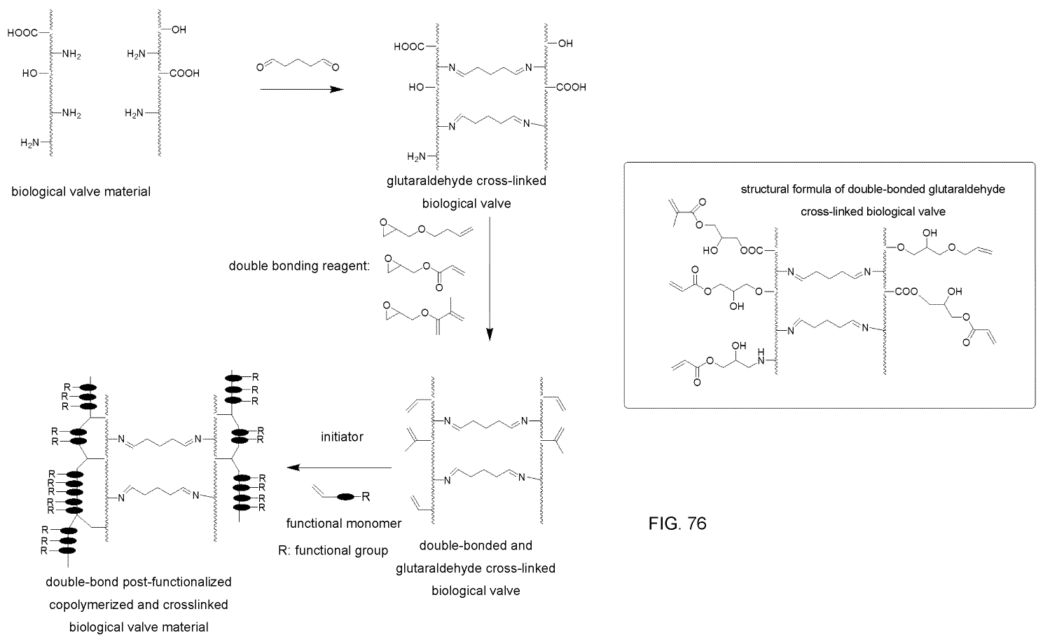

For ease understanding of the chemical mechanism involved in this scheme, FIG. 76 is illustrated as an example for explanation. The double bonding reagent (first functional monomer) is used to modify the glutaraldehyde cross-linked biological valve material. A ring-opening reaction is occurred between the oxirane group of the the double bonding reagent (i.e., the first functional monomer) and the hydroxyl groups, carboxyl groups as well as the remaining small amount of amino groups after glutaraldehyde cross-linking on the glutaraldehyde cross-linked biological valve material, thereby introducing carbon-carbon double bonds to the glutaraldehyde cross-linked biological valve material. Further, carbon-carbon double bonds and functional groups B are further introduced through physical permeation of a second functional monomer. Furthermore, a polymerization between the double bonds on the biological valve material and the double bonds of the functional monomer is initiated to introduce a functionalized polymer cross-linked network and to achieve secondary cross-linking, thereby completing the post-functionalized copolymerization and cross-linking treatment of double bonds of the biological valve material.

Since more functional groups (hydroxyl groups and carboxyl groups other than amino groups) on the biological valve material are used for cross-linking, and the polymer of functional monomer is further introduced as a cross-linked network through copolymerization with the functional monomer, the biological valve material has a larger cross-linked network, the degree of cross-linking of the biological valve material with double bonds suffered from post-functionalized copolymerization and cross-linking is significantly improved, and its structural stability and anti-calcification performance are also significantly improved with the introduction of functionalized polymer network. Furthermore, the functionalized polymer network is introduced to the biological valve through post-functionalized copolymerization of double bonds, so that the biological valve material is rich in functional groups, thus endowing the biological valve material with properties of the functional groups. The functional group B can be selected from hydroxyl group, carboxyl group, choline carboxylate, choline sulfonate, choline phosphate, pyrrolidone, sulfonic acid group, carboxylate ion, sulfonate, sulfoxide, amide group, and methoxy group. These groups can bind with water molecules through hydrogen bonds and ion hydration, which further enhances the hydrophilicity of the surface of the biological valve material and forms a hydration layer on the biological valve to prevent excessive adhesion of proteins and cells in the human body, improving anti-thrombotic properties and biocompatibility.

For the introduced functional group B:
hydroxyl group: as a hydrophilic group, it improves the surface hydrophilicity of the biological material to achieve anticoagulant effect;
carboxyl group: as a hydrophilic group, it improves the surface hydrophilicity of the biological material to achieve anticoagulant effect;
carboxylate ion and sulfonic acid group: it improves the surface hydrophilicity of the biological material through ion hydration to achieve anticoagulant effect;
sulfoxide and pyrrolidone: as hydrophilic groups, they improve the surface hydrophilicity of the biological material to achieve anticoagulant effect;
zwitterion: it improves the surface hydrophilicity of the biological material through ion hydration to achieve anti-coagulant effect, and it is beneficial to form an electrically neutral surface of the biological valve, thereby reducing the adsorption of calcium ions to achieve anti-calcification effect;
polyethylene glycol: as a hydrophilic group, it improves the surface hydrophilicity of the biological material, and increases the steric hindrance between calcium ions and collagen, improving the surface hydrophilicity of the biological valve material;
carbamate group and urea group: as hydrophilic groups, they improve the surface hydrophilicity of the biological material to achieve anticoagulant effect;
carbamate group: as a hydrophilic group, it improves the surface hydrophilicity of the biological material to achieve anticoagulant effect; and
amide: as a hydrophilic group, it improves the surface hydrophilicity of the biological material to achieve anticoagulant effect; as a toughening group, it can dynamically adjust the elasticity of the biological material to improve biological material utilization; and the resulted valve has excellent hydrodynamic properties.

In step BS120 of this scheme, the double bonding reagent (first functional monomer) selection, the concentration of the double bonding reagent (first functional monomer) solution, the solvent selection of the double bonding reagent (first functional monomer) solution, the reaction time and operation process for double bonding modification are the same as those in Scheme 6, which will not be repeated here.

In step BS130 of this scheme:
the biological valve material treated in step BS120 is soaked in the solution containing the second functional monomer directly or after cleaning;
optionally, the second functional monomer has at least one second carbon-carbon double bond and at least one functional group B;
optionally, the second functional monomer is one or more of acrylamide, acrylic acid, sodium acrylate, methacrylic acid, sodium methacrylate, 2-(prop-2-enoylamino)acetic acid, 2-acrylamido-2-methylpropanesulfonic acid, hydroxyethyl methacrylate, 3-[[2-(methacryloyloxy)ethyl]dimethylammonio]propionate, N-methyl-2-acrylamide, N-isopropylacrylamide, N-(hydroxymethyl)acrylamide, N-(2-hydroxyethyl)methacrylamide, 3-[N,N-dimethyl-[2-(2-methylprop-2-enoyloxy)ethyl]ammonium]propane-1-sulfonate, 2-methacryloyloxyethyl phosphorylcholine, N-(2-hydroxyethyl)acrylamide, N-(methoxymethyl)methacrylamide, 2-acrylamido-2-methylpropanesulfonic acid, 2-acrylamido-2-methylpropanesulfonic acid, and double-bonded hyaluronic acid (which can be prepared by the above-mentioned method);
optionally, the concentration of the solution containing the second functional monomer is in the range of 0.1% to 6% (v/v).
optionally, the solvent of the solution containing the second functional monomer is one or more of water, physiological saline, ethanol, isopropanol or neutral pH buffer solution;
optionally, the soaking time in the solution containing the second functional monomer is in the range of 0.5 h to 120 h.

In step S200 of this scheme:
the step S200 of this scheme is the same as the step S200 of the scheme 7, and will not be repeated here.

Compared with the prior art, this scheme has at least one of the following benefits:
(1) this scheme modifies the biological valve material on the basis of glutaraldehyde cross-linking; carbon-carbon bonds are introduced to the glutaraldehyde cross-linked biological valve material by reaction between the double bonding reagent and the glutaraldehyde cross-linked biological valve material; the resulted double-bonded glutaraldehyde cross-linked biological valve material serves as the basis for functionalized copolymerization and cross-linking; further, a polymerization between the carbon-carbon double bonds on the glutaraldehyde cross-linked biological valve material and the carbon-carbon double bonds of the functional monomer is further initiated to introduce polymers of functional monomer as the functionalized cross-linked network to achieve functionalized copolymerization and cross-linking, which can further improve the cross-linking degree of the biological valve material and introduce functional groups; by increasing the degree of cross-linking, the stability of the biological valve material will be improved;
(2) this scheme introduces carbon-carbon double bonds to the glutaraldehyde cross-linked biological valve material, and further initiates the polymerization between the carbon-carbon double bonds on the double-bonded biological valve material and the carbon-carbon double bonds of the functional monomer to introduce a functionalized polymer cross-linked network on the biological valve material; this functionalized cross-linked network can serve as a polymer barrier to reduce the contact and interaction between collagenase in the human body and the collagen matrix on the biological valve material, significantly reducing the degradation effect of collagenase on collagen matrix of the biological valve material, improving the stability of the glutaraldehyde cross-linked biological valve material, and further reducing the risk of structural degradation of the biological valve caused by structural degradation of the biological valve material;
(3) this scheme introduces double bonds to the glutaraldehyde cross-linked biological valve material, and further initiates the polymerization between the carbon-carbon double bonds on the double-bonded and glutaraldehyde cross-linked biological valve material and the carbon-carbon double bonds of the functional monomer to introduce a functionalized polymer cross-linked network, which can serve as a polymer barrier to further reduce the binding of calcium ions to the mineralized areas on the biological valve material that are prone to binding with calcium ions, reducing the risk of calcification and thus playing an anti-calcification role;
(4) this scheme introduces carbon-carbon double bonds to the glutaraldehyde cross-linked biological valve material, and further initiates the polymerization between the carbon-carbon double bonds on the double-bonded and glutaraldehyde cross-linked biological valve material and the carbon-carbon double bonds of the functional monomer to introduce a functionalized polymer cross-linked network; by introducing the functionalized polymer cross-linked network, the degree of cross-linking of the biological valve material is increased, resulting in a more rigid structure of the biological valve material and increasing the elasticity; moreover, the functionalized polymer cross-linked network fills the gaps between the collagen matrices on the biological valve material to inhibit the deformation of collagen fibers, making the biological valve material harder while increasing the elasticity;
(5) this scheme introduces carbon-carbon double bonds to the glutaraldehyde cross-linked biological valve material, and further initiates the polymerization between the carbon-carbon double bonds on the double-bonded and glutaraldehyde cross-linked biological valve material and the carbon-carbon double bonds of the functional monomer to introduce a functionalized polymer cross-linked network; since the functionalized polymer cross-linked network further contains functional groups, both the re-crosslinking and the functionalization of the biological valve material are achieved; the biological valve material suffered from functionalized copolymerization and post-crosslinking has functional groups and exhibits properties of the functional groups; the functional group can be selected from hydroxyl group, carboxyl group, choline carboxylate, choline sulfonate, choline phosphate, pyrrolidone, sulfonic acid group, carboxylate ion, sulfonate, sulfoxide, amide group, and methoxy group, which can bind with water molecules through hydrogen bonds and ion hydration, which further enhances the hydrophilicity of the surface of the biological valve material and forms a hydration layer on the biological valve to prevent excessive adhesion of proteins and cells in the human body, improving anti-thrombotic properties and biocompatibility.

In Schemes 6 to 8, all reactions in BS100 and BS200 can be carried out at 0 to 50° C. unless otherwise specified. Preferably, the temperature does not need to be specially controlled and can be at room temperature. It is suitable not to exceed the human body's adaptive temperature, preferably between 36 and 37° C.

In Schemes 6 to 8, all reactions in BS100 and BS200 can be either static reactions or dynamic reactions unless otherwise specified. Dynamic reactions can be conducted using equipment capable of circulating the solution, such as a peristaltic pump, or through shaking at a rotation speed ranging from 10 rpm to 150 rpm. The peristaltic cycle or shaking can be carried out continuously or intermittently.

In BS110 of Schemes 6 to 8, the concentration of the glutaraldehyde solution is 0.1% to 5% (w/w), and the cross-linking time can be any time in the range of 0.5 h to 120 h.

For Schemes 6 to 8, optionally, dehydration and drying treatment after the double bond polymerization is completed are carried out to form a dry valve. After the double bond polymerization is completed, the biological valve material is conventionally cleaned, softened, and then dehydrated and dried.

The cleaning solution can be one or more of water, physiological saline, ethanol, isopropanol or neutral pH buffer solution. The pH can be adjusted to between 5.0 and 9.5 before and during use, or there is no need to adjust the pH.

Optionally, the dehydration treatment involves exposing the valve piece subjected to double bond polymerization or the valve formed by sewing valve pieces to a dehydration solution.

Optionally, the dehydration solution is a mixed solution of alcohol solution and water, with the alcohol solution accounting for 20-90% (v/v). The alcohol reagent can be one or both of ethanol and isopropanol.

Optionally, the drying treatment involves exposing the dehydrated valve piece or valve to a softener solution for 20 minutes to 10 hours.

Optionally, the main component of the softener solution is one or a mixed solution of glycerol and polyethylene glycol, wherein the glycerol concentration is in the range of 10-100% (v/v), and the remaining component is one or more of water, ethanol, isopropanol, etc., accounting for 0-90% (v/v).

Optionally, the valve sterilization method after drying treatment can be one of ethylene oxide sterilization or electron beam sterilization.

The biological valve material prepared by the above methods can be used for interventional biological valves, for example, through minimally invasive intervention; or be used for surgical biological valves, for example, through surgical implantation.

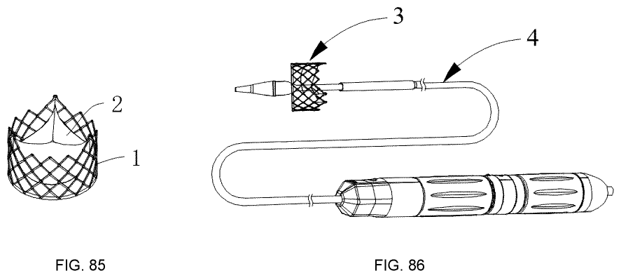

As shown in FIG. 85, a prosthetic heart valve according to an embodiment includes a stent 1 and leaflets 2 connected in the stent 1. The stent is generally cylindrical, and the side wall thereof has a hollow mesh structure. There is a blood flow channel in the stent, and the leaflets cooperate with each other to control the opening and closing of the blood flow channel in the stent.

Depending on the release modes, the stent is made of corresponding materials during processing, such as nickel-titanium alloy that has shape memory and can self-expand in the human body, or stainless steel that is released by ball expansion, etc. The stent itself can be formed by cutting pipes or braiding wires. The leaflets can be connected to the stent by sewing, bonding or integral molding.

For positioning in the human body, positioning structures that can interact with surrounding native tissues can be provided on the periphery of the stent, such as anchors, arms, etc. In order to prevent peripheral leakage, skirts or anti-peripheral leakage materials or the like can be provided on the inside and/or outside of the stent. The leaflets, skirts or anti-peripheral leakage materials can all be made of the biological valve materials in the above embodiments.

As shown in FIG. 86, in case of intervention through catheter, the prosthetic heart valve 3 and a corresponding delivery system form a valve intervention system. The delivery system includes a catheter assembly 4 and a handle for controlling the catheter assembly. The prosthetic heart valve is in a radially compressed state when being delivered in the human body, and released and radially expanded in the human body after releasing the restriction of the catheter assembly or being expanded by ball expansion.

Specific examples are illustrated in the following.

### Example 1

In this example, freshly collected porcine pericardium was washed with distilled water for 2 hours at 4° C. with shaking at 100 RPM, and then soaked in 30 mM DL-2-amino-4-pentenoic acid aqueous solution at 37° C. for 12 hours. Then glutaraldehyde was added to a concentration of 100 mM, wherein the porcine pericardium was soaked for 24 hours at 37° C. with shaking at 100 RPM. Then, the porcine pericardium was washed with distilled water. Thereafter, the porcine pericardium was soaked in 5% polyethylene glycol diacrylate aqueous solution at 37° C. for 12 hours to ensure sufficient physical permeation of polyethylene glycol diacrylate. An initiator of ammonium persulfate and sodium bisulfite was added to initiate the reaction for 24 hours at 37° C., wherein the molar concentrations of ammonium persulfate and sodium bisulfite were both 40 mM. The sample obtained was recorded as sample 1.

Meanwhile, a glutaraldehyde treated porcine pericardium was set as control group 1, wherein the pericardium was soaked in 0.625% glutaraldehyde for 24 hours.

### Control Example 1

Freshly collected porcine pericardium was washed with distilled water for 2 hours at 4° C. with shaking at 100 RPM until there is no visible adherent non-pericardium or non-collagenous tissue. Then the cleaned biological material was soaked in 0.625% glutaraldehyde 37° C. for 24 hours to prepare a biological valve material, recorded as control example 1.

### Chemical Structure Determination

The biological valve material (GA-PEG) of the above-mentioned Example 1 and the biological valve material (GA) of the Control example 1 were arbitrarily selected for infrared spectrum analysis. The samples of the above-mentioned Example 1 and the Control example 1 were cut into 1 cm×1 cm and freeze-dried, and analyzed by attenuated total reflection infrared spectroscopy (Thermo Scientific Nicolet iS50 FT-IR, USA), with a scanning wavelength of 4000-400 cm⁻¹.

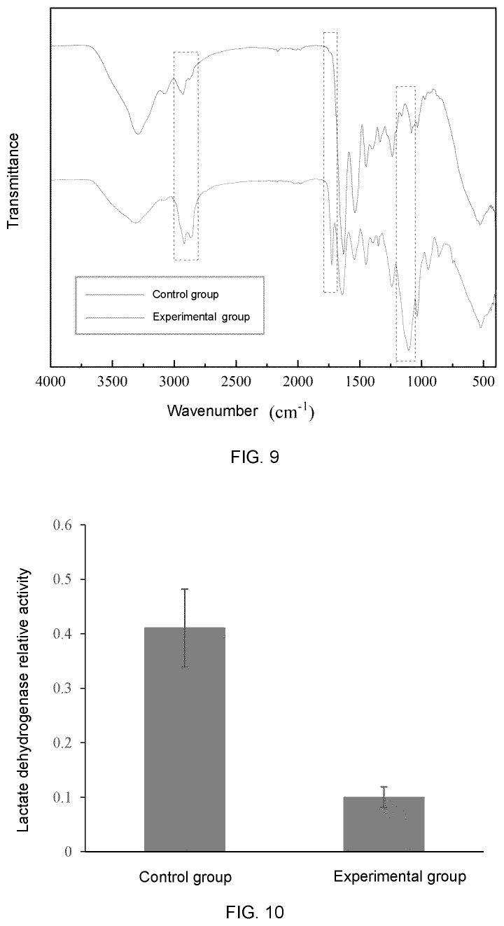

The infrared spectra of the pericardium (GA) of sample 1 and control group 1 are shown in FIG. 9. The C—H stretching vibration peaks of —CH2- at 2919 cm⁻¹ and 2867 cm⁻¹, the C═O stretching vibration peak of the ester at 1724 cm⁻¹, and the C—O stretching vibration peak of the ether bond at 1100 cm⁻¹ exhibit evident intensity in the infrared spectrum of Example 1. It can be seen that polyethylene glycol is successfully chemically grafted on the biological valve material.

### Lactate Dehydrogenase Relative Activity Measurement

In order to determine the anticoagulant properties of the biological valve materials, the biological valve materials of the above-mentioned Example 1 and Control example 1 were selected to measure the lactate dehydrogenase relative activity.

The measurement method is as follows: the sample to be measured (circular sheet with a diameter of 6 mm) was rinsed with 0.9% physiological saline for 5 minutes, incubated with 100 μL platelet-rich plasma in a 96-well plate for 1 hour at 37° C.; then the serum was aspirated and the sample surface was washed three times with PBS for five minutes for each time. The positive control was 100 μL platelet-rich plasma. A lactate dehydrogenase assay kit (Beyotime Biotechnology, Shanghai, China) was used according to the instructions to detect the relative amount of platelets adhered to the sample surface. The absorbance value at 490 nm was measured using a microplate reader (BioTek Synergy H1, USA).

### Calcium Content Determination

In order to determine the binding force of biological valve materials with calcium ions and thus to determine the anti-calcification properties of different biological valve materials, the biological valve materials of the above-mentioned Example 1 and Control example 1 were arbitrarily selected for calcium content determination.

The measurement method of calcium content is as follows: the sample to be measured (size of 1 cm×1 cm) was rinsed with 0.9% physiological saline for 5 minutes; samples were surgically implanted in two subcutaneous pockets in the central dorsal wall region of 45-50 g male Sprague-Dawley rats (one sample/rat/group); after 30 days, the implanted samples were removed from the rat's dorsal wall; after removing the fibrous capsule around the sample, it was freeze-dried and weighed as dry weight; thereafter, calcium ions were extracted using 6 M hydrochloric acid (Adamas, Shanghai, China) in a 95° C. water bath for 24 hours, and the calcium content was measured using an inductively coupled atomic emission spectrometer (Agilent 720).

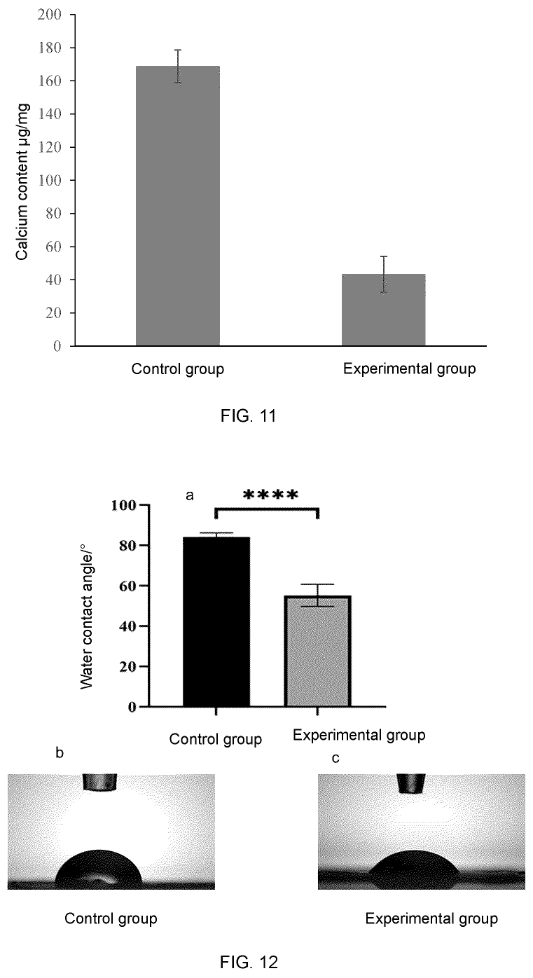

The measurement results of the lactate dehydrogenase relative activity in Example 1 and glutaraldehyde control group 1 are shown in FIG. 10, the analysis results are shown in Table 1, and the measurement results of the calcium content are shown in FIG. 11 and Table 2.

TABLE 1

| | Lactate dehydrogenase relative activity |
|---|---|
| Glutaraldehyde control group 1 | 0.410 ± 0.072 |
| Example 1 | 0.100 ± 0.019 |

TABLE 2

| | Calcium content μg/mg |
|---|---|
| Glutaraldehyde control group 1 | 168.595 ± 9.973 |
| Example 1 | 43.220 ± 10.873 |

### Stability Determination

The biological valve material (GA-PEG) of the above-mentioned Example 1 and the biological valve material (GA) of the Control example 1 were arbitrarily selected for enzyme degradation test. The samples of the above-mentioned Example 1 and the Control example 1 were cut into circular sheets with a diameter of 1 cm, freeze-dried, and weighed as dry weight. Each sample was soaked in 500 μL of Tris buffer (0.1 M Tris, 50 mM CaCl₂, pH=7.4) of 100 U/mL type I collagenase (Invitrogen, NY, USA) at 37° C. for 2 to 4 hours. After soaking, the samples were washed three times with deionized water, freeze-dried and weighed as dry weight to calculate the weight loss rate of the samples.

The analysis results are as follows.

The weight loss rate (%) of Example 1: 3.234±0.125; the weight loss rate (%) of Control example 1: 8.036±0.760.

The weight loss rate of Control Example 1 under the action of type I collagenase for 24 hours reached 8.0%, while the weight loss rate of Example 1 was only 3.2%, indicating that the structural stability of the biological valve material prepared in the present disclosure is significantly improved.

### Example 2

Preparation of modified hyaluronic acid: 2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Preparation of modified polylysine: polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

In this example, freshly collected porcine pericardium was washed with distilled water for 2 hours at 4° C. with shaking at 100 RPM, and then soaked in 180 mM modified polylysine aqueous solution at room temperature for 12 hours. Then, glutaraldehyde solution was added to a mass concentration of 2.5%. After reaction on a shaker at 37° C. for 24 hours, the pericardium material was taken out and cleaned, and soaked in a 50 mg/ml modified hyaluronic acid aqueous solution at room temperature for 12 hours, and then soaked in 2.5% ammonium persulfate and 0.25% N,N,N′,N′-tetramethylethylenediamine at 37° C. for 12 hours, and finally washed with distilled water, recorded as sample 2.

The sample 2 prepared in Example 2 and the sample prepared in control group 2 were respectively subjected to water contact angle test, lactate dehydrogenase activity test, hemolysis rate test and calcification test.

Control group 2: freshly collected porcine pericardium was washed with distilled water for 2 hours at 4° C. with shaking at 100 RPM, and then soaked in a glutaraldehyde solution with a mass concentration of 0.625% for 24 hours, and then taken out and soaked in a glutaraldehyde solution with a mass fraction of 0.2% and stored, which was recorded as control sample 2.

### (1) Water Contact Angle Test

The materials from the control group and Example 1 were cut into square sheets of 1*1 cm, placed between two pieces of glass and flatten, and freeze-dried in a vacuum for water contact angle test.

(2) Lactate dehydrogenase activity test: fresh rabbit blood was collected and centrifuged at 1500 rpm for 15 minutes to obtain platelet-rich plasma; the materials from the control group and Example 1 were cut into circular sheets with a diameter of 10 mm and washed three times with PBS, and placed into a 48-well plate; 100 μL of platelet-rich plasma was added to soak the sample at 37° C. for 1 hour; 100 μL of platelet-rich plasma was used as a positive control for quantitative detection; after incubation, the sample was rinsed 3 times with PBS; the relative amount of platelet adhesion was determined using a lactate dehydrogenase assay kit; a microplate reader was used to record the absorbance value of each group at 490 nm, and the lactate dehydrogenase relative activity in each group was calculated. The relative number of platelets is expressed by the lactate dehydrogenase relative activity.

### (3) Hemolysis Rate Test

Fresh rabbit blood was collected and centrifuged at 1500 rpm for 15 min, with the supernatant discarded, and the red blood cells remained. Samples from the control group and Example 1 were placed into 2 ml centrifuge tube, with PBS diluted red blood cells (9/1, PBS/RBC) added for incubation at 37° C. for 1 hour. Red blood cells diluted 10 times with PBS and deionized water were used as negative and positive controls respectively. The sample was centrifuged at 3000 rpm for 5 min, with the supernatant transferred to a 96-well plate. A microplate reader was used to record the absorbance value at 545 nm and the hemolysis rate was calculated.

### (4) Calcification Test

An incision is made on the back of a 45-50 g male SD rat, and a blunt instrument was used to separate the subcutaneous tissue to create a cavity. The samples from the control group and the Example 1 were placed into the cavity, then the skin was sutured. After 30 days, the samples were taken out and freeze-dried and weighed, and digested with 1 ml of 6 M hydrochloric acid at 100° C. Thereafter, the digested solution was diluted to 10 ml with deionized water, and the calcium concentration was determined through inductively coupled plasma atomic emission spectrometry.

The water contact angle test results are shown in a to c of FIG. 12. The control group is the glutaraldehyde treated group (i.e., the Control Example 2), and the experimental group is the hydrophilic treatment group (i.e., Example 2). The water contact angle of the experimental group decreased. Further, the final water contact angle results of Example 2 and glutaraldehyde control group 2 are shown in Table 3.

TABLE 3

| | Water contact angle (°) |
|---|---|
| Glutaraldehyde control group 2 | 84.29 |
| Example 2 | 55.26 |

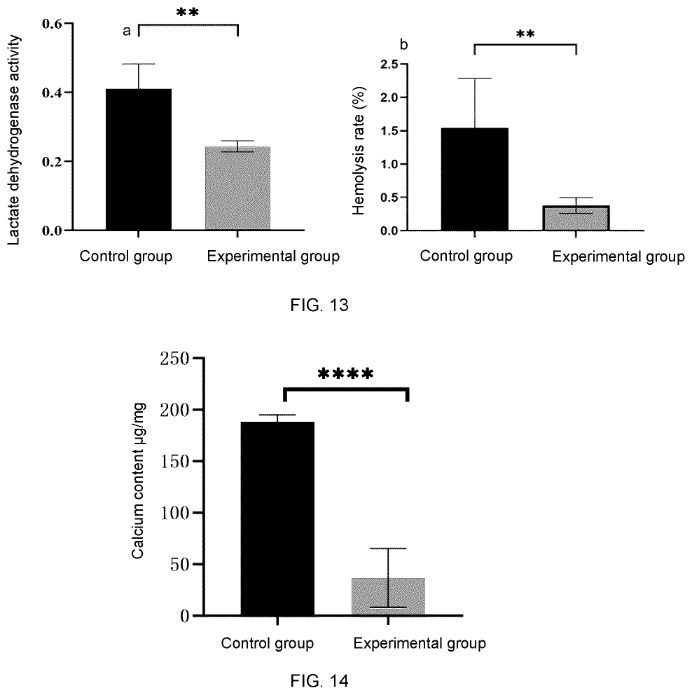

The test results of lactate dehydrogenase activity and hemolysis rate are shown in FIG. 13, wherein a is the comparison result of lactate dehydrogenase activity, b is the comparison result of hemolysis rate, c is the comparison result of calcium ion concentration. The control group is the glutaraldehyde treated group (i.e., the Control Example 2), and the experimental group is the hydrophilic treatment group (i.e., Example 2). Both the lactate dehydrogenase activity and hemolysis rate of the experimental group are reduced. Further, the final lactate dehydrogenase activity and hemolysis rate results of Example 2 and glutaraldehyde control group 2 are shown in Table 4.

TABLE 4

| | Lactate dehydrogenase | Hemolysis |
|---|---|---|
| | activity | rate (%) |
| Glutaraldehyde control group 2 | 0.41 | 1.54 |
| Example 2 | 0.24 | 0.38 |

The calcium ion concentration test results are shown in FIG. 14. The control group is the glutaraldehyde treated group (i.e., the Control Example 2), and the experimental group is the hydrophilic treatment group (i.e., Example 2). The calcium ion content of the experimental group decreased. Further, the final calcium ion concentration results of Example 2 and glutaraldehyde control group 2 are shown in Table 5.

TABLE 5

| | Calcium ion concentration (μg/mg) |
|---|---|
| Glutaraldehyde control group 2 | 188.39 |
| Example 2 | 36.95 |

Referring to Table 1, Table 2 and Table 3, it can be concluded that after the biological material is treated with the method of Example 2, the water contact angle of the biological material decreases, the lactate dehydrogenase activity decreases, and the calcium ion content decreases.

The method according to the present example can improve the hydrophilic properties, blood compatibility and anti-calcification ability of the biological material, potentially extending its lifespan.

### Control Group 3

Freshly collected porcine pericardium was washed with distilled water for 2 hours at 4° C. with shaking at 100 RPM, then soaked in 100 mM glutaraldehyde solution and cross-linked for 24 hours at room temperature with shaking at 100 RPM to obtain control sample 3.

### Example 3

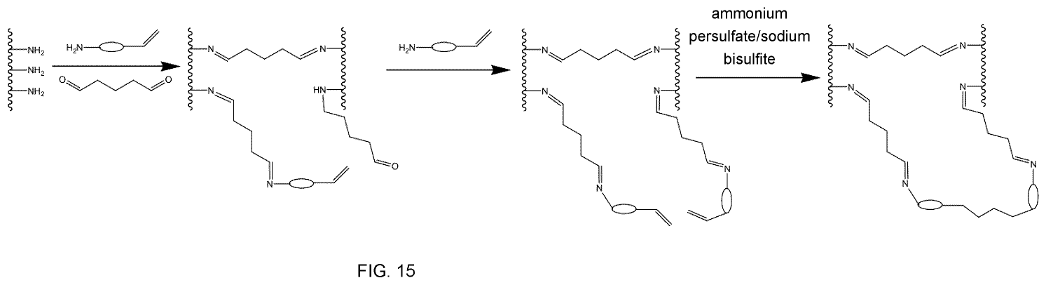

The preparation process is shown in FIG. 3, in which a method for preparing a biological valve material by co-crosslinking and double-bond cross-linking is shown. The basic principle is shown in FIG. 15. Freshly collected porcine pericardium was washed with distilled water for 2 hours at 4° C. with shaking at 100 RPM, and then soaked in 20 mM 2-methylallylamine aqueous solution at 37° C. for 2 hours. Then glutaraldehyde was added to a final concentration of 100 mM, wherein the porcine pericardium was soaked for 24 hours at 37° C. with shaking at 100 RPM. Then, the porcine pericardium was taken out and washed with distilled water. Thereafter, the porcine pericardium was soaked in 20 mM 2-methylallylamine aqueous solution at 37° C. for 4 hours. Then the porcine pericardium was taken out and washed with distilled water. After cleaning, the porcine pericardium was soaked in distilled water, and an initiator of ammonium persulfate and sodium bisulfite was added to initiate the reaction for 24 hours at 37° C., wherein the molar concentrations of ammonium persulfate and sodium bisulfite were both 30 mM. In order to distinguish the samples prepared in different examples, the sample obtained in this example was recorded as sample 3.

### Example 4

Freshly collected porcine pericardium was washed with distilled water for 2 hours at 4° C. with shaking at 100 RPM, and then soaked in 20 mM 3-butene-1-amine aqueous solution at 37° C. for 2 hours. Then glutaraldehyde was added to a final concentration of 100 mM, wherein the porcine pericardium was soaked for 24 hours at 37° C. with shaking at 100 RPM. Then, the porcine pericardium was taken out and washed with distilled water. Thereafter, the porcine pericardium was soaked in deionized water, and an initiator of ammonium persulfate and sodium bisulfite was added to initiate the reaction for 24 hours at 37° C., wherein the molar concentrations of ammonium persulfate and sodium bisulfite were both 30 mM. The sample obtained in this example was recorded as sample 4.

### Example 5

Freshly collected porcine pericardium was washed with distilled water for 2 hours at 4° C. with shaking at 100 RPM, and then soaked in 20 mM 2-aminoethyl methacrylate aqueous solution at 37° C. for 2 hours. Then glutaraldehyde was added to a final concentration of 100 mM, wherein the porcine pericardium was soaked for 24 hours at 37° C. with shaking at 100 RPM. Then, the porcine pericardium was taken out and washed with distilled water. Thereafter, the porcine pericardium was soaked in deionized water, and an initiator of ammonium persulfate and sodium bisulfite was added to initiate the reaction for 24 hours at 37° C., wherein the molar concentrations of ammonium persulfate and sodium bisulfite were both 30 mM. The sample obtained in this example was recorded as sample 5.

### Example 6

Freshly collected porcine pericardium was washed with distilled water for 2 hours at 4° C. with shaking at 100 RPM, and then soaked in an aqueous solution containing 10 mM 2-aminoethyl methacrylate and 10 mM 2-methylallylamine at 37° C. for 2 hours. Then, glutaraldehyde was added to a final concentration of 100 mM, wherein the porcine pericardium was soaked for 24 hours at 37° C. with shaking at 100 RPM. Then, the porcine pericardium was taken out and washed with distilled water. Thereafter, the porcine pericardium was soaked in deionized water, and an initiator of potassium persulfate and sodium bisulfite in equal molar amounts was added to initiate the polymerization reaction for 24 hours at 37° C., wherein the molar concentrations of ammonium persulfate and sodium bisulfite are both 30 mM. Then, the porcine pericardium was washed with distilled water and transferred to glycerol for dehydration to obtain a dry valve sample. The sample obtained in this example was recorded as sample 6.

### Example 7

Freshly collected porcine pericardium was washed with distilled water for 2 hours at 4° C. with shaking at 100 RPM, and then soaked in 20 mM 2-aminoethyl methacrylate aqueous solution at 37° C. for 2 hours. Then glutaraldehyde was added to a final concentration of 100 mM, wherein the porcine pericardium was soaked for 24 hours at 37° C. with shaking at 100 RPM. Then, the porcine pericardium was taken out and washed with distilled water. Thereafter, the porcine pericardium was soaked in 20 mM 2-amino-4-pentenoic acid aqueous solution at 37° C. for 24 hours. Then the porcine pericardium was taken out and washed with distilled water, and then soaked in distilled water, in which an initiator of ammonium persulfate and sodium bisulfite was added to initiate the reaction for 24 hours at 37° C., wherein the molar concentrations of ammonium persulfate and sodium bisulfite were both 30 mM. The sample obtained in this example was recorded as sample 7.

### Example 8

Freshly collected porcine pericardium was washed with distilled water for 2 hours at 4° C. with shaking at 100 RPM, and then soaked in 20 mM 2-aminoethyl methacrylate aqueous solution at 37° C. for 2 hours. Then glutaraldehyde was added to a final concentration of 100 mM, wherein the porcine pericardium was soaked for 24 hours at 37° C. with shaking at 100 RPM. Then, an initiator of ammonium persulfate and sodium bisulfite was added directly to the solution to initiate the reaction for 24 hours at 37° C., wherein the molar concentrations of ammonium persulfate and sodium bisulfite were both 30 mM. The sample obtained in this example was recorded as sample 8.

### Example 9

Preparation of modified hyaluronic acid: 2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Preparation of modified polylysine: polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

In this example, freshly collected porcine pericardium was washed with distilled water for 2 hours at 4° C. with shaking at 100 RPM, and then soaked in 60 mM (calculated as lysine) modified polylysine aqueous solution at room temperature for 12 hours. Then, glutaraldehyde solution was added to a mass concentration of 250 mM. After reaction on a shaker at 37° C. for 24 hours, the pericardium material was taken out and cleaned, and soaked in a 50 mg/ml modified hyaluronic acid aqueous solution at room temperature for 12 hours, and then soaked in 2.5% ammonium persulfate and 0.25% N,N,N′,N′-tetramethylethylenediamine at 37° C. for 12 hours, and finally washed with distilled water, recorded as sample 9. Compared with control sample 2, the water contact angle of sample 9 decreased, the lactate dehydrogenase activity decreased, and the calcium ion content decreased, which can improve the hydrophilic properties, blood compatibility and anti-calcification ability of the biological material, potentially extending its lifespan.

### Enzyme Degradation Experiment (Characterization of Crosslinking Degree):

Sample 5, sample 6, sample 7, sample 8 and control group 3 were cut into circular sheets with a diameter of 1 cm, with each group consisting of 6 parallel samples. All circular sheet samples were placed on 48-well plate, frozen overnight at −80° C., and then transferred to a vacuum freeze-dryer to freeze-dry for 48 hours. Each sample was weighed on a microbalance as the initial weight (WO) and placed back into the 48-well plate. 0.5 mL collagenase I solution in PBS was added with a pipette to the wells of the 48-well plate so that the biological valve sample is completely immersed in the collagenase solution in PBS (100 U/mL). Then the 48-well plate was transferred to a 37° C. constant temperature incubator for incubation for 2 to 4 hours. After the incubation, the solution in the well plate was discarded, and a dropper with a rubber bulb was used to draw deionized water to repeatedly flush the biological valve sample in the well plate. After repeated flushing three times, the sample was frozen overnight at −80° C. and then transferred to a vacuum freeze-dryer to freeze-dry for 48 hours. Each sample after degradation by collagenase solution was weighed on a microbalance as the final weight (Wt). The calculation formula for enzymatic degradation weight loss rate is as follows:

$$Enzymatic degradation weight loss rate = \frac{{W}_{0} - {W}_{t}}{{W}_{0}} \times 100 \%$$

TABLE 6

| Sample | Enzymatic degradation weight loss rate (%) |
|---|---|
| Control group 3 | 8.31 |
| Sample 5 | 5.27 |
| Sample 6 | 4.88 |
| Sample 7 | 4.11 |
| Sample 8 | 3.23 |

The enzyme degradation experiment was conducted on samples 5, 6, 7, 8 and control group 3 to characterize the cross-linking efficiency of samples, and the enzymatic degradation weight loss rates of samples after sample 5, sample 6, sample 7, sample 8 and control group 3 were treated with collagenase I were then calculated as shown in Table 6. The enzymatic degradation weight loss rates of sample 5, sample 6, sample 7, and sample 8 are all lower than that of the control group 3, which indicates that sample 5, sample 6, sample 7, and sample 8 all have higher enzymatic degradation stability than the control group 3, that is, the cross-linking efficiency of sample 5, sample 6, sample 7 and sample 8 is higher. The results of the enzyme degradation experiment show that the co-crosslinking and double-bond cross-linking method of the present disclosure for preparing biological valve materials can improve the cross-linking degree of biological valve materials.

An alizarin red staining experiment was conducted on the samples obtained in Control Group 3 and Examples 3 to 8 as follows.

Sample 3, sample 4, sample 5, sample 6, sample 7, sample 8 and control group 3 were implanted into the subcutaneous tissue of rats for 30 days and then taken out and fixed with paraformaldehyde tissue fixative. After fixation, the sampled were taken out and smoothed with a scalpel, and then transferred to a dehydration box. The material samples were dehydrated using gradients of 50%, 75%, 85%, 95% (v/v) and absolute ethanol. After dehydration, the material samples were transferred to an embedding machine and embedded in melted wax, and then transferred to a −20° C. refrigerator to cool and trimmed. Slices with 3-5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner.

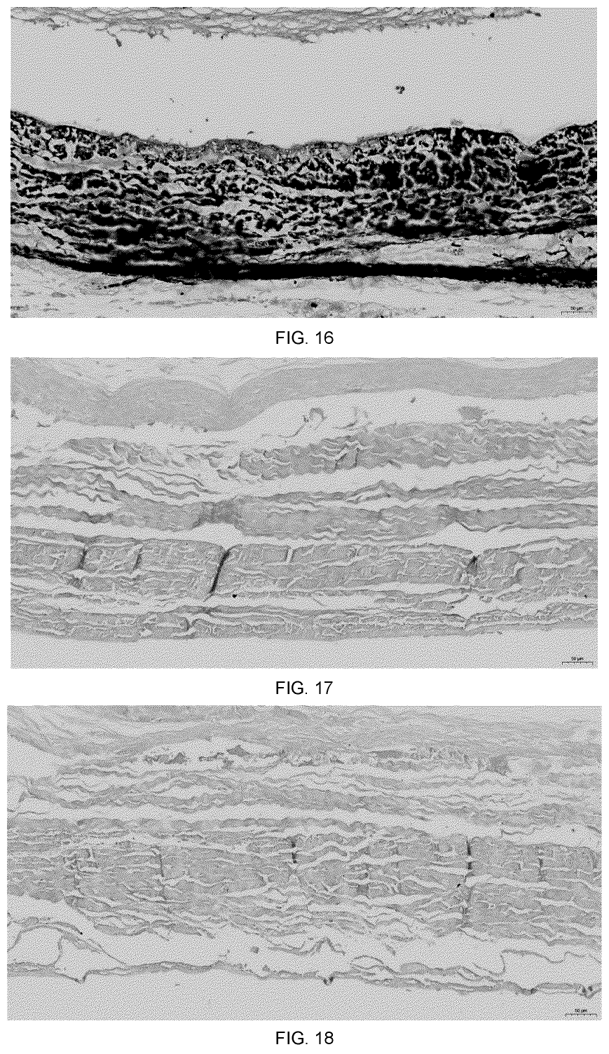

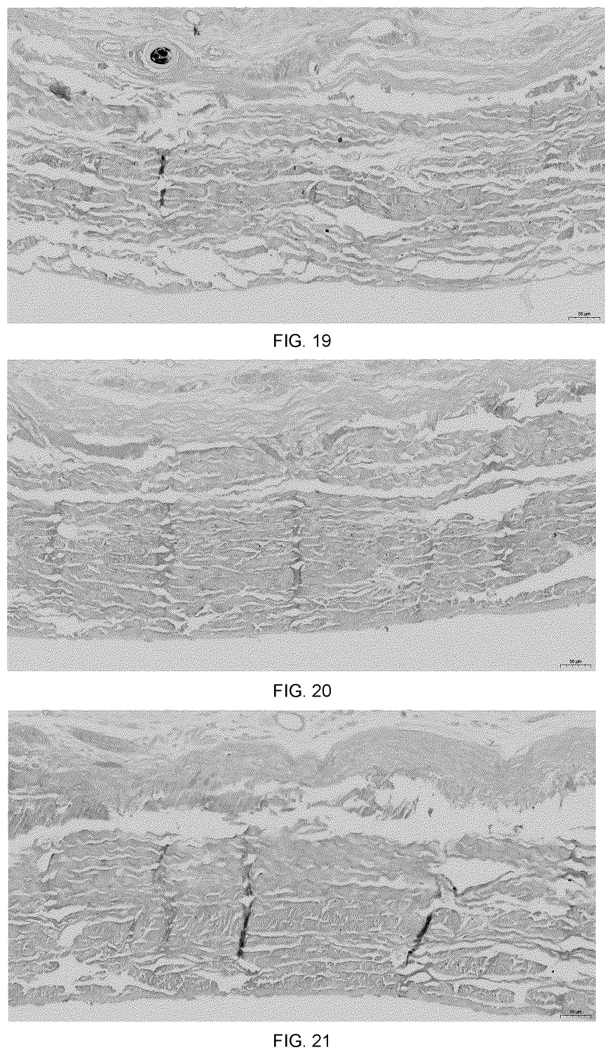

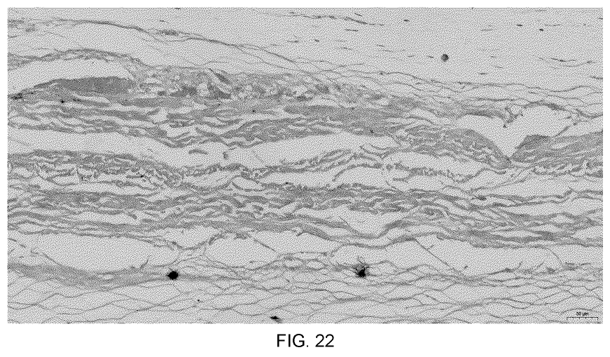

The alizarin red staining experiment was conducted on sample 3, sample 4, sample 5, sample 6, sample 7, sample 8 and control group 3 after being implanted subcutaneously in rats for 30 days to characterize the degree of calcification of samples. Images of the alizarin red staining results of sample slices after being implanted subcutaneously in rats for 30 days are shown in FIGS. 16-22, wherein the darker the color of the sample after alizarin red staining, the higher the degree of calcification. Compared with the alizarin red staining result of slice of control sample 3 (FIG. 16), the color of the alizarin red staining results of slices of sample 3, sample 4, sample 5, sample 6, sample 7 and sample 8 is obviously lighter, which indicates that the degree of calcification of sample 3, sample 4, sample 5, sample 6, sample 7 and sample 8 is lower than that of control group 3, that is, compared with control group 3, sample 3, sample 4, sample 5, sample 6, sample 7 and sample 8 have certain anti-calcification effect. The alizarin red staining results of sample 3, Sample 4, sample 5, sample 6, sample 7, sample 8 and control group 3 after being implanted subcutaneously in rats for 30 days show that the co-crosslinking and double-bond cross-linking method of the present disclosure for preparing biological valve materials can improve the anti-calcification performance of biological valves.

### Control Group 4

Freshly collected porcine pericardium was washed with distilled water for 2 hours at 4° C. with shaking at 100 RPM, then soaked in 100 mM glutaraldehyde solution and cross-linked for 24 hours at room temperature with shaking at 100 RPM to obtain control sample 4.

### Example 10

Freshly collected porcine pericardium was washed with distilled water for 2 hours at 4° C. with shaking at 100 RPM;

Then the porcine pericardium was soaked in 30 mM 2-amino-7-en-octanoic acid solution at 37° C. for 4 hours, and then glutaraldehyde was added to a final concentration of 100 mM, wherein the porcine pericardium was soaked for 24 hours at 37° C. with shaking at 100 RPM;

The porcine pericardium was taken out and washed with distilled water;

The porcine pericardium was soaked in 30 mM 2-amino-7-en-octanoic acid solution for 4 hours;

After cleaning, the porcine pericardium was soaked in deionized water, and an initiator of ammonium persulfate and sodium bisulfite was added to initiate the reaction for 24 hours at 37° C., wherein the molar concentrations of ammonium persulfate and sodium bisulfite are both 30 mM. The sample obtained was recorded as sample 10.

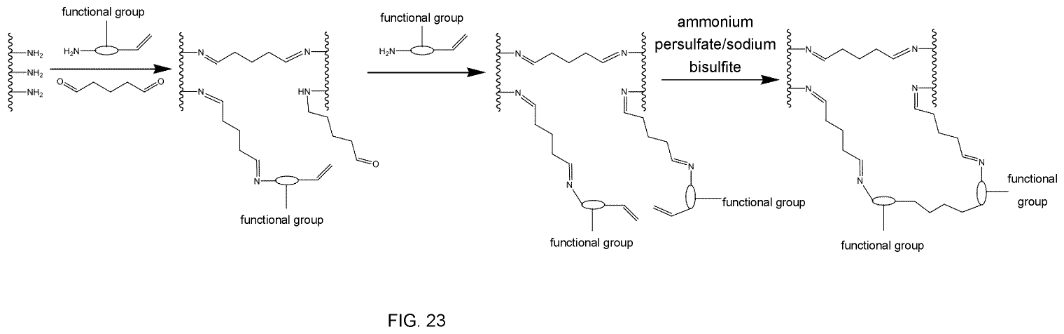

The reaction schematic diagram of this example is shown in FIG. 23.

### Example 11

Freshly collected porcine pericardium was washed with distilled water for 2 hours at 4° C. with shaking at 100 RPM;

Then the porcine pericardium was soaked in 20 mM 4-(1-amino-2-methyl-propyl)-hepta-1,6-diene-4-ol aqueous solution at 37° C. for 24 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

The porcine pericardium was soaked in 30 mM 4-(1-amino-2-methyl-propyl)-hepta-1,6-diene-4-ol aqueous solution for 4 hours;

After cleaning, the porcine pericardium was soaked in deionized water, and an initiator of ammonium persulfate and sodium bisulfite was added to initiate the reaction for 24 hours at 37° C., wherein the molar concentrations of ammonium persulfate and sodium bisulfite are both 30 mM. The sample obtained was recorded as sample 11.

### Example 12

Freshly collected porcine pericardium was washed with distilled water for 2 hours at 4° C. with shaking at 100 RPM;

Then the porcine pericardium was soaked in 20 mM 2-aminopent-4-enoic acid aqueous solution at 37° C. for 4 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

After cleaning, the porcine pericardium was soaked in deionized water, and an initiator of ammonium persulfate and sodium bisulfite was added to initiate the reaction for 24 hours at 37° C., wherein the molar concentrations of ammonium persulfate and sodium bisulfite are both 30 mM; and then the porcine pericardium was transferred to glycerol for dehydration to obtain a dry valve sample recorded as sample 12.

### Example 13

Freshly collected porcine pericardium was washed with distilled water for 2 hours at 4° C. with shaking at 100 RPM, and then soaked in 20 mM 2-aminopent-4-enoic acid aqueous solution at 37° C. for 4 hours;

Then glutaraldehyde was added to a final concentration of 100 mM, wherein the porcine pericardium was soaked for 24 hours at 37° C. with shaking at 100 RPM;

An initiator of ammonium persulfate and sodium bisulfite was added to initiate the reaction for 24 hours at 37° C., wherein the molar concentrations of ammonium persulfate and sodium bisulfite are both 30 mM. The sample obtained was recorded as sample 13.

Enzyme degradation experiment (characterization of crosslinking degree):

The enzyme degradation experiment was conducted on sample 12, sample 13 and control group 4 using the same method as the aforementioned enzyme degradation experiment. The results are shown in Table 7.

TABLE 7

| | Enzymatic degradation weight loss rate |
|---|---|
| Control group 4 | 7.06% |
| Sample 12 | 5.81% |
| Sample 13 | 4.53% |

The enzyme degradation experiment was conducted on samples 12, 13 and control group 4 to characterize the cross-linking degree of samples, and the enzymatic degradation weight loss rates of samples after sample 12, sample 13 and control group 4 were treated with collagenase I were then calculated as shown in Table 7. The enzymatic degradation weight loss rates of samples 12 and 13 are both lower than that of the control group 4, which indicates that samples 12 and 13 have higher enzymatic degradation stability than the control group 4, that is, the cross-linking degree of samples 12 and 13 is higher. The results of the enzyme degradation experiment show that the double-bond post-crosslinking method for preparing biological valve materials in this example can improve the cross-linking degree of biological valve materials.

### Example 14

Freshly collected porcine pericardium was washed with distilled water for 2 hours at 4° C. with shaking at 100 RPM, and then soaked in 20 mM 2-aminopent-4-enoic acid aqueous solution at 37° C. for 4 hours;

Then glutaraldehyde was added to a final concentration of 100 mM, wherein the porcine pericardium was soaked for 24 hours at 37° C. with shaking at 100 RPM;

The porcine pericardium was transferred to a 50 mM 2-aminopent-4-enoic acid aqueous solution and soaked for 4 hours;

An initiator of ammonium persulfate and sodium bisulfite was added to initiate the reaction for 24 hours at 37° C., wherein the molar concentrations of ammonium persulfate and sodium bisulfite are both 30 mM. The sample obtained was recorded as sample 14.

### Example 15

Freshly collected porcine pericardium was washed with distilled water for 2 hours at 4° C. with shaking at 100 RPM, and then soaked in 20 mM 2-aminopent-4-enoic acid aqueous solution at 37° C. for 4 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

The porcine pericardium was soaked in 50 mM 2-aminopent-4-enoic acid aqueous solution for 4 hours;

The porcine pericardium was taken out and washed with distilled water;

The pericardium was soaked in distilled water, in which an initiator of ammonium persulfate and sodium bisulfite was added to initiate the reaction for 24 hours at 37° C., wherein the molar concentrations of ammonium persulfate and sodium bisulfite were both 30 mM; and then the pericardium was washed with distilled water and dehydrated with glycerol to obtain a dry valve sample which was recorded as sample 15.

### Example 16

Preparation of modified hyaluronic acid: 2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Preparation of modified polylysine: polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

In this example, freshly collected porcine pericardium was washed with distilled water for 2 hours at 4° C. with shaking at 100 RPM, and then soaked in 60 mM (calculated as lysine) modified polylysine aqueous solution at room temperature for 12 hours. Then, glutaraldehyde solution was added to a mass concentration of 250 mM. After reaction on a shaker at 37° C. for 24 hours, the pericardium material was taken out and cleaned, and soaked in a 50 mg/ml modified hyaluronic acid aqueous solution at room temperature for 12 hours, and then soaked in 2.5% ammonium persulfate and 0.25% N,N,N′,N′-tetramethylethylenediamine at 37° C. for 12 hours, and finally washed with distilled water, recorded as sample 16. Compared with control sample 2, the water contact angle of sample 16 decreased, the lactate dehydrogenase activity decreased, and the calcium ion content decreased, which can improve the hydrophilic properties, blood compatibility and anti-calcification ability of the biological material, potentially extending its lifespan.

The samples obtained in Control example 4 and Examples 10 to 15 (sample 10, sample 11, sample 12, sample 13, sample 14, sample 15 and control sample 4) were subjected to a subcutaneous implantation experiment in rats. After 30 days of implantation, samples were taken out and subjected to an alizarin red staining experiment to characterize the degree of calcification of samples after 30 days of subcutaneous implantation in rats.

### Alizarin Red Staining Experiment:

The alizarin red staining experiment was conducted on samples 10, 11, 12, 13, 14, 15 and control sample 4 using the same method as the aforementioned alizarin red staining experiment.

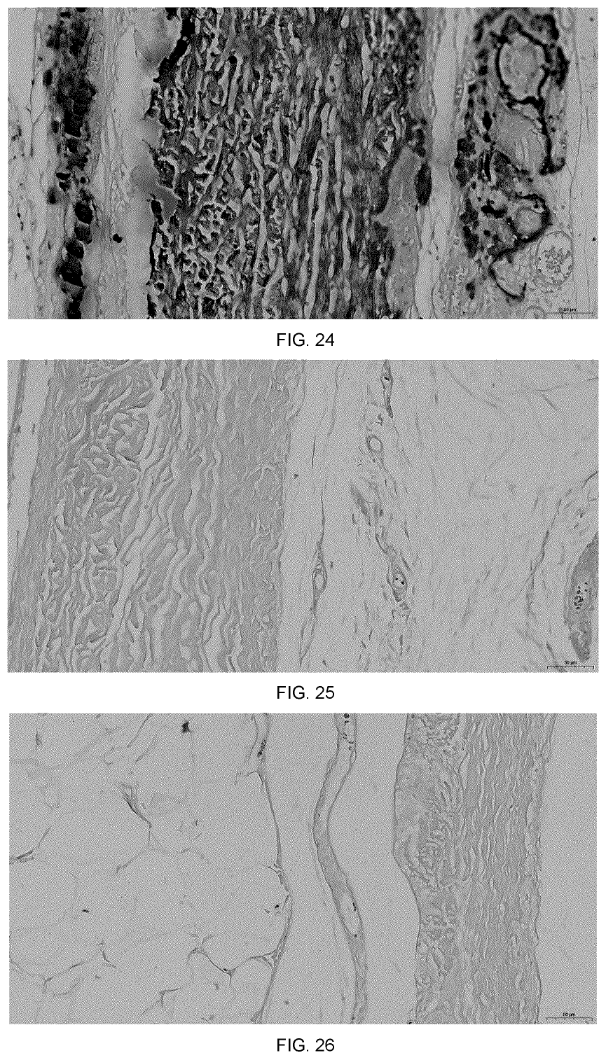

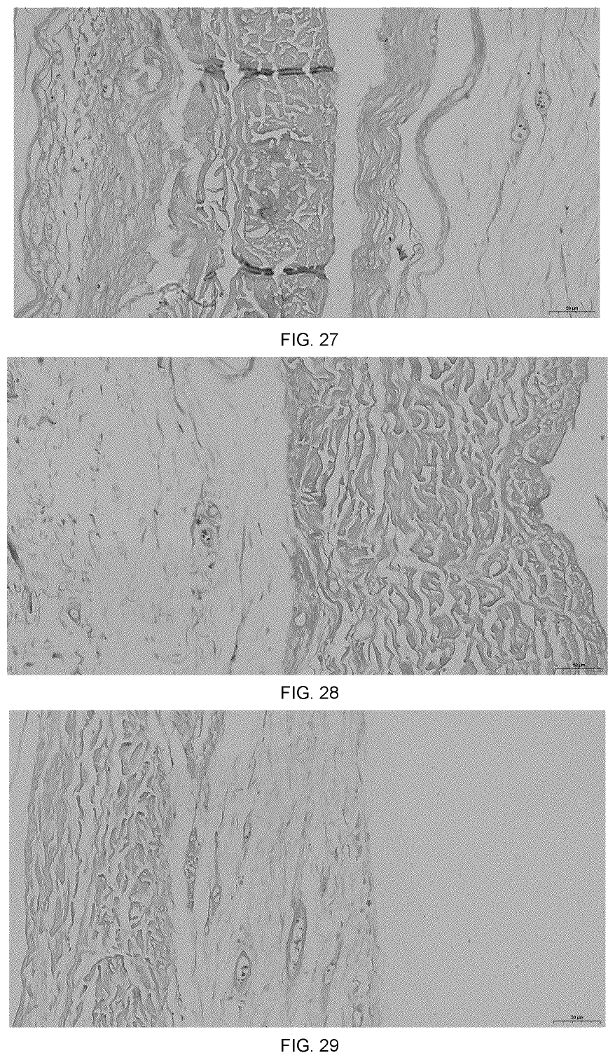

Samples 10, 11, 12, 13, 14, 15 and control sample 4 after being implanted subcutaneously in rats for 30 days were stained through the alizarin red staining experiment to characterize the degree of calcification of samples. Images of the alizarin red staining results of sample slices of control sample 4 and samples 10, 11, 12, 13, 14 and 15 after being implanted subcutaneously in rats for 30 days are shown in FIGS. 24 to 30, wherein the darker the color of the sample after alizarin red staining, the higher the degree of calcification. Compared with the alizarin red staining result of slice of control sample 4 (FIG. 24), the color of the alizarin red staining results of samples 10, 11, 12, 13, 14, and 15 is obviously lighter, which indicates that the degree of calcification of sample 10, sample 11, sample 12, sample 13, sample 14, and sample 15 is lower than that of control sample 3, that is, compared with control group 4, sample 10, sample 11, sample 12, sample 13, sample 14, and sample 15 have certain anti-calcification effect. The alizarin red staining results of sample 10, sample 11, sample 12, sample 13, sample 14, sample 15 and the control group 4 after being implanted subcutaneously in rats for 30 days show that the double-bond post-crosslinking method for preparing biological valve materials in this example can improve the anti-calcification performance of biological valves.

### Blood Contact Experiment:

Control sample 4 and samples 12 and 13 with uniform surface and thickness were cut into sheets with a diameter of 1 cm, which were rinsed with physiological saline, drained and placed in a 24-well plate. 300 μL rabbit blood was added to the wells for incubation at 37° C. for 1 hour with shaking at 70 bpm. After the incubation, the rabbit blood was discarded and 500 μL of physiological saline was added to the wells to wash away the non-adherent blood with gentle shaking on the shaker. After washing, the samples were transferred to 2.5% (w/w) glutaraldehyde solution for fixation for 4 hours. The fixed samples were dehydrated using gradient ethanol (25%, 50%, 75% and 100%, v/v), with each gradient lasting 20 minutes. The dried samples were fixed on the test stand with conductive glue and sprayed with gold. Images of blood adhesion on the samples were observed and photographed on a field emission scanning electron microscope.

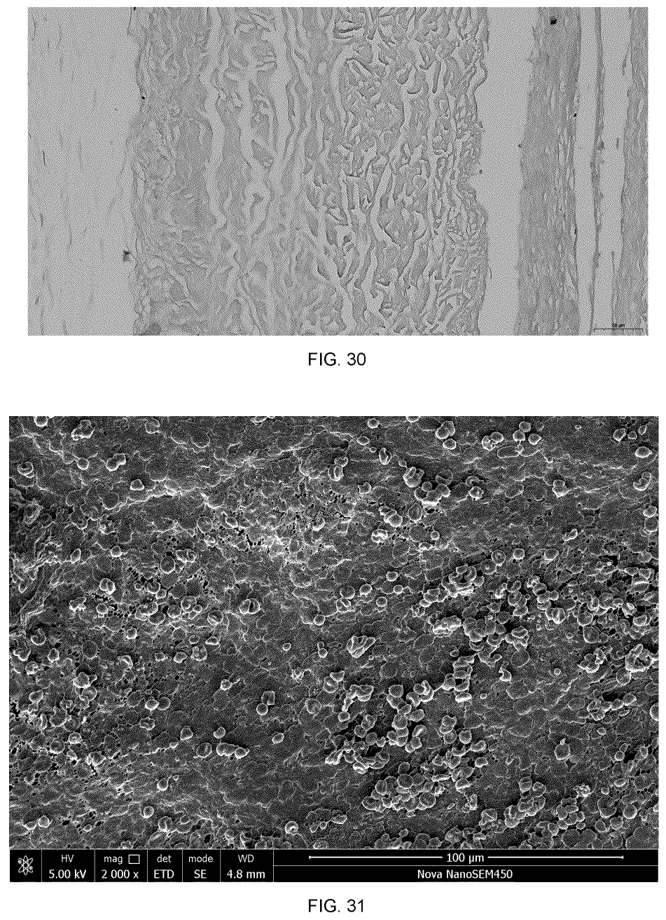

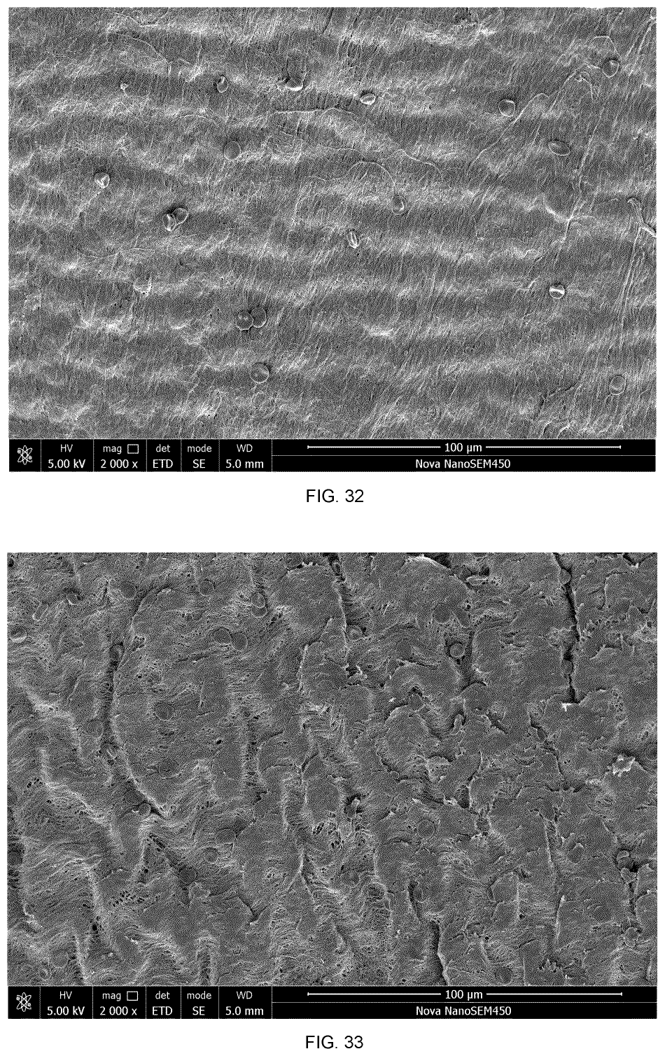

The scanning electron microscope images of the blood contact experiment of control sample 4 and samples 12 and 13 are shown in FIGS. 31 to 33. After contact and incubation with rabbit blood, more adherent and aggregated blood cells were observed in the scanning electron microscope image of control sample 4, while samples 12 and 13 had fewer adherent blood cells, with only a few blood cells dispersedly adhered to the surface. The results show that samples 12 and 13 can inhibit the adhesion of blood cells to a certain extent, thereby reducing the risk of coagulation, and have anticoagulant effects.

### Control Group 5

Freshly collected porcine pericardium was washed with distilled water for 2 hours at 4° C. with shaking at 100 RPM, then soaked in 100 mM glutaraldehyde solution and cross-linked for 24 hours at room temperature with shaking at 100 RPM.

### Example 17

Freshly collected porcine pericardium was washed with distilled water for 2 hours at 4° C. with shaking at 100 RPM;

Then the porcine pericardium was soaked in 20 mM 2-methylallylamine aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

The porcine pericardium was soaked in 20 mM 2-methylallylamine aqueous solution for 2 hours;

After cleaning, the porcine pericardium was soaked in 5 wt % N,N′-ethylenebisacrylamide aqueous solution at 37° C. for 12 hours to ensure sufficient physical permeation of N,N′-ethylenebisacrylamide;

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 17.

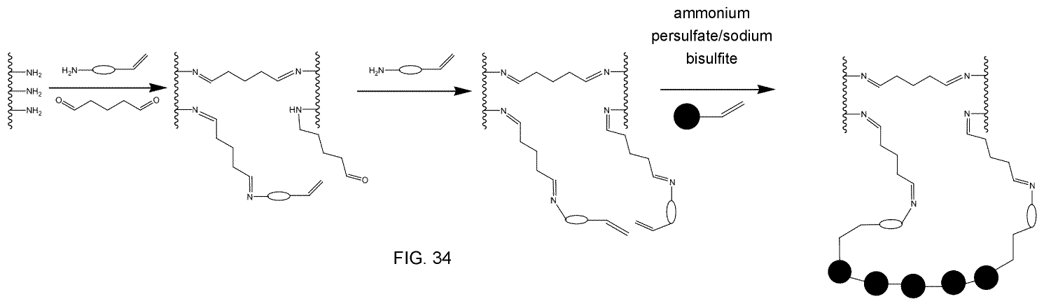

The reaction schematic diagram of this example is shown in FIG. 34.

### Example 18

Freshly collected porcine pericardium was washed with distilled water for 2 hours at 4° C. with shaking at 100 RPM;

Then the porcine pericardium was soaked in 20 mM 2-methylallylamine aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

After cleaning, the porcine pericardium was soaked in a 5 wt % 1,4-butanediol diacrylate aqueous solution at 37° C. for 12 hours to ensure sufficient physical permeation of 1,4-butanediol diacrylate;

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 18.

### Example 19

Freshly collected porcine pericardium was washed with distilled water for 2 hours at 4° C. with shaking at 100 RPM;

Then the porcine pericardium was soaked in 20 mM 2-aminopent-4-enoic acid aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

After cleaning, the porcine pericardium was soaked in 2 wt % (ethane-1,2-diylbis(oxy))bis(ethane-2,1-diyl)diacrylate aqueous solution at 37° C. for 12 hours to ensure sufficient physical permeation of (ethane-1,2-diylbis(oxy))bis(ethane-2,1-diyl)diacrylate;

An initiator of ammonium persulfate and sodium bisulfite was added to initiate the reaction for 24 hours at 37° C., wherein the molar concentrations of ammonium persulfate and sodium bisulfite are both 30 mM;

The porcine pericardium was washed with distilled water, soaked in glycerol and dehydrated to obtain a dry valve. In order to distinguish the samples prepared in different examples, the sample obtained in this example was recorded as sample 19.

### Example 20

Freshly collected porcine pericardium was washed with distilled water for 2 hours at 4° C. with shaking at 100 RPM;

Then the porcine pericardium was soaked in 20 mM 2-aminoethyl methacrylate aqueous solution at 37° C. for 2 hours;

Then glutaraldehyde was added to a final concentration of 100 mM, wherein the porcine pericardium was soaked for 24 hours at 37° C. with shaking at 100 RPM;

N,N′-ethylenebisacrylamide was added to a final concentration of 5%, wherein the porcine pericardium was soaked at 37° C. for 12 hours to ensure sufficient physical permeation of N,N′-ethylenebisacrylamide.

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 20.

### Example 21

Freshly collected porcine pericardium was washed with distilled water for 2 hours at 4° C. with shaking at 100 RPM;

Then the porcine pericardium was soaked in 20 mM 2-aminopent-4-enoic acid aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

The pericardium was soaked in 50 mM 2-aminopent-4-enoic acid aqueous solution for 4 hours;

After cleaning, the porcine pericardium was soaked in 2 wt % (ethane-1,2-diylbis(oxy))bis(ethane-2,1-diyl)diacrylate aqueous solution at 37° C. for 12 hours to ensure sufficient physical permeation of (ethane-1,2-diylbis(oxy))bis(ethane-2,1-diyl)diacrylate;

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 21.

### Example 22

Preparation of modified hyaluronic acid: 2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Preparation of modified polylysine: polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

In this example, freshly collected porcine pericardium was washed with distilled water for 2 hours at 4° C. with shaking at 100 RPM, and then soaked in 60 mM (calculated as lysine) modified polylysine aqueous solution at room temperature for 12 hours. Then, glutaraldehyde solution was added to a mass concentration of 250 mM. After reaction on a shaker at 37° C. for 24 hours, the pericardium material was taken out and cleaned, and soaked in a 50 mg/ml modified hyaluronic acid aqueous solution at room temperature for 12 hours, and then soaked in 2.5% ammonium persulfate and 0.25% N,N,N′,N′-tetramethylethylenediamine at 37° C. for 12 hours, and finally washed with distilled water, recorded as sample 22. Compared with control sample 2, the water contact angle of sample 22 decreased, the lactate dehydrogenase activity decreased, and the calcium ion content decreased, which can improve the hydrophilic properties, blood compatibility and anti-calcification ability of the biological material, potentially extending its lifespan.

### Enzyme Degradation Experiment:

The collagenase degradation weight loss rates of sample obtained in Control example 5 and sample 17, sample 18, sample 19, sample 20, and sample 21 obtained in Examples 17-20 were measured using the same method as the aforementioned enzyme degradation experiment.

The results are shown in Table 8. From the results in Table 8, it can be seen that the preparation method of the present disclosure can significantly improve the cross-linking degree of the biological material.

TABLE 8
Collagenase degradation weight loss rate

| Sample No. | Collagenase degradation weight loss rate |
|---|---|
| Control group 5 | 8.6% |
| Sample 17 | 2.3% |
| Sample 18 | 3.7% |
| Sample 19 | 1.9% |
| Sample 20 | 3.8% |
| Sample 21 | 3.1% |

The enzyme degradation experiment was conducted on samples 17, 18, 19, 20, 21 and control group 5 to characterize the cross-linking degree of samples, and the enzymatic degradation weight loss rates of samples after samples 17, 18, 19, 20, 21 and control group 5 were treated with collagenase I were calculated as shown in Table 8. The enzymatic degradation weight loss rates of sample 17, sample 18, sample 19, sample 20, and sample 21 were all lower than that of the control group 5, indicating that sample 17, sample 18, sample 19, and sample 20 have higher enzymatic degradation stability than the control group 5, that is, the cross-linking degree of sample 17, sample 18, sample 19, and sample 20 is higher. The results of the enzyme degradation experiment show that the co-crosslinking and double-bond post-crosslinking method for preparing biological valve materials of the present disclosure can improve the cross-linking degree of biological valve materials.

### Alizarin Red Staining Experiment:

The alizarin red staining experiment was conducted on sample 17, sample 18, sample 19, sample 20, sample 21 and control group 5 using the same test method as the aforementioned alizarin red staining experiment.

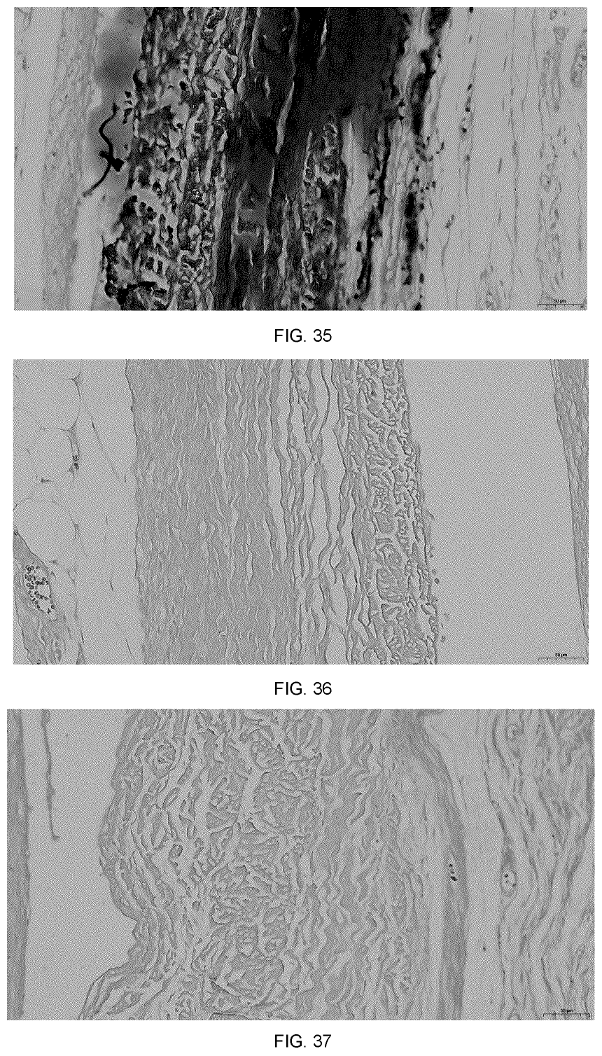

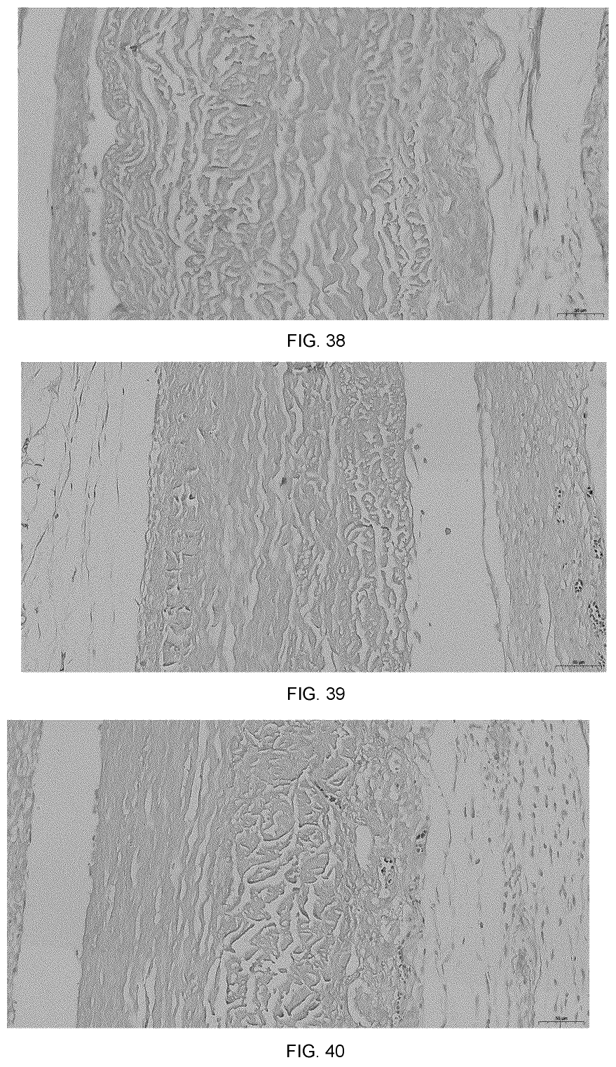

The control group 5, sample 17, sample 18, sample 19, sample 20, and sample 21 after being implanted subcutaneously in rats for 30 days were stained through the alizarin red staining experiment to characterize the degree of calcification of samples. Images of the alizarin red staining results of the sample slices after being implanted subcutaneously in rats for 30 days are shown in FIGS. 35 to 40, wherein the darker the color of the sample after alizarin red staining, the higher the degree of calcification. Compared with the alizarin red staining result of slice of control sample 5 (FIG. 35), the color of the alizarin red staining results of samples 17, 18, 19, 20, and 21 is obviously lighter, which indicates that the degree of calcification of sample 17, sample 18, sample 19, sample 20, and sample 21 is lower than that of control sample 5, that is, compared with control group 5, sample 17, sample 18, sample 19, sample 20, and sample 21 have certain anti-calcification effect. The alizarin red staining results of sample 17, sample 18, sample 19, sample 20, sample 21 and control group 5 after being implanted subcutaneously in rats for 30 days show that the co-crosslinking and double-bond post-crosslinking method for preparing biological valve materials of the present disclosure can improve the anti-calcification performance of biological valves.

### Control Group 6

Freshly collected porcine pericardium was washed with distilled water for 2 hours at 4° C. with shaking at 100 RPM, then soaked in 100 mM glutaraldehyde solution and cross-linked for 24 hours at room temperature with shaking at 100 RPM to obtain control sample 6.

### Example 23

Freshly collected porcine pericardium was washed with distilled water for 2 hours at 4° C. with shaking at 100 RPM;

Then the porcine pericardium was soaked in 30 mM 2-aminopent-4-enoic acid aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

The porcine pericardium was soaked in 30 mM 2-aminopent-4-enoic acid aqueous solution for 2 hours;

After cleaning, the porcine pericardium was soaked in 5 wt % 3-[N,N-dimethyl-[2-(2-methylprop-2-enoyloxy)ethyl]ammonium]propane-1-sulfonate aqueous solution at 37° C. for 12 hours to ensure sufficient physical permeation of 3-[N,N-dimethyl-[2-(2-methylprop-2-enoyloxy)ethyl]ammonium]propane-1-sulfonate;

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 23.

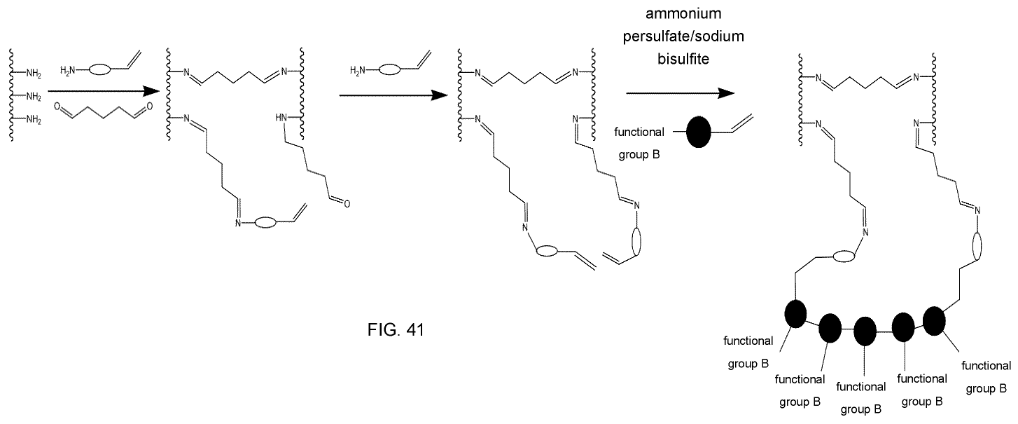

The reaction schematic diagram of this example is shown in FIG. 41.

### Example 24

Freshly collected porcine pericardium was washed with distilled water for 2 hours at 4° C. with shaking at 100 RPM;

Then the porcine pericardium was soaked in 20 mM 2-aminopent-4-enoic acid aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

After cleaning, the porcine pericardium was soaked in 3 wt % 3-[[2-(methacryloyloxy)ethyl]dimethylammonio]propionate aqueous solution at 37° C. for 12 hours to ensure sufficient physical permeation of 3-[[2-(methacryloyloxy)ethyl]dimethylammonio]propionate.

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 24.

### Blood Contact Experiment

Blood contact experiment was conducted on control sample 6, sample 23 and sample 24 using the same test method as the aforementioned blood contact experiment.

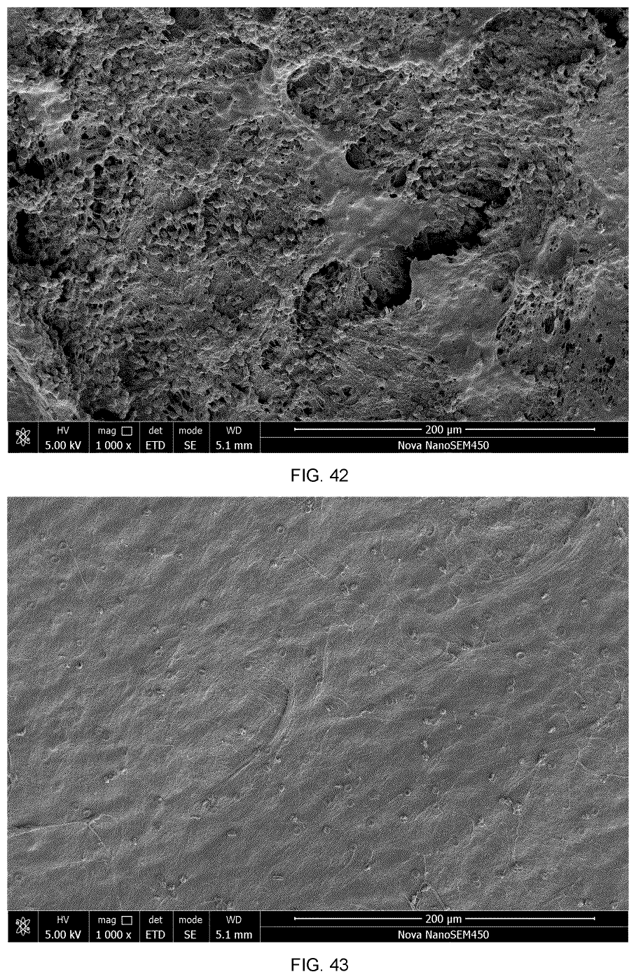

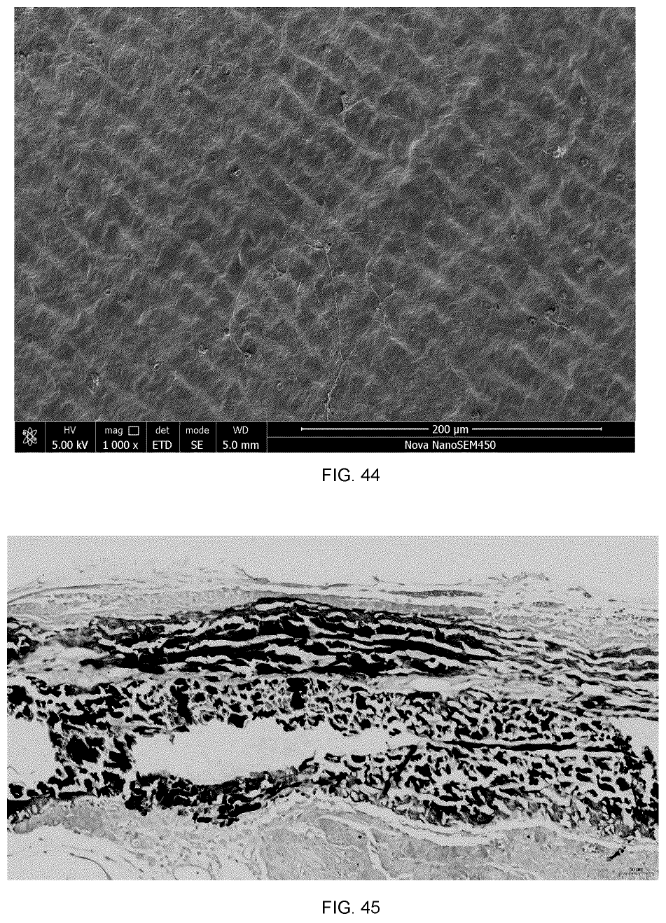

The scanning electron microscope images of the blood contact experiment of control sample 6, sample 23 and sample 24 are shown in FIG. 42, FIG. 43 and FIG. 44. After contact and incubation with rabbit blood, more adherent and aggregated blood cells were observed in the scanning electron microscope image of control sample 6, while samples 23 and 24 had fewer adherent blood cells, with only a few blood cells dispersedly adhered to the surface. The results show that samples 23 and 24 can inhibit the adhesion of blood cells to a certain extent, thereby reducing the risk of coagulation, and have anticoagulant effects.

### Example 25

Freshly collected porcine pericardium was washed with distilled water for 2 hours at 4° C. with shaking at 100 RPM;

Then the porcine pericardium was soaked in 20 mM 2-aminoethyl methacrylate aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

After cleaning, the porcine pericardium was soaked in 5 wt % N-isopropylacrylamide aqueous solution at 37° C. for 12 hours to ensure sufficient physical permeation of N-isopropylacrylamide;

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 25.

### Enzyme Degradation Experiment (Characterization of Crosslinking Degree)

The collagenase degradation weight loss rates of sample 23, sample 24, sample 25 and control group 6 were measured using the same method as the aforementioned enzyme degradation experiment.

The results are shown in Table 9. From the results in Table 9, it can be seen that the preparation method of the present disclosure can significantly improve the cross-linking degree of the biological material.

TABLE 9
Collagenase degradation weight loss rate

| Sample No. | Collagenase degradation weight loss rate |
|---|---|
| Control group 6 | 8.6% |
| Sample 23 | 2.9% |
| Sample 24 | 1.7% |
| Sample 25 | 2.4% |

The enzyme degradation experiment was conducted on sample 23, sample 24, sample 25 and control group 6 to characterize the cross-linking degree of samples, and the enzymatic degradation weight loss rates of samples after sample 23, sample 24, sample 25 and control group 6 were treated with collagenase I were then calculated as shown in the table above. The enzymatic degradation weight loss rates of samples 23, 24, and 25 were all lower than that of the control group 6, which indicates that sample 23, sample 24, and sample 25 have higher enzymatic degradation stability than the control group 6, that is, samples 23, 24, and sample 25 has a higher degree of cross-linking. The results of the enzyme degradation experiment show that the method for preparing biological valve materials of the present disclosure can improve the cross-linking degree of biological valve materials.

### Example 26

Freshly collected porcine pericardium was washed with distilled water for 2 hours at 4° C. with shaking at 100 RPM;

Then the porcine pericardium was soaked in 20 mM 2-aminoethyl methacrylate aqueous solution at 37° C. for 2 hours;

Then glutaraldehyde was added to a final concentration of 100 mM, wherein the porcine pericardium was soaked for 24 hours at 37° C. with shaking at 100 RPM;

N-isopropylacrylamide was added to a final concentration of 5 wt %, wherein the porcine pericardium was soaked for 12 hours at 37° C. to ensure sufficient physical permeation of N-isopropylacrylamide.

An initiator of ammonium persulfate and sodium bisulfite was added to initiate the reaction for 24 hours at 37° C., wherein the molar concentrations of ammonium persulfate and sodium bisulfite are both 30 mM. In order to distinguish the samples prepared in different examples, the sample obtained in this example was recorded as sample 26.

### Example 27

Freshly collected porcine pericardium was washed with distilled water for 2 hours at 4° C. with shaking at 100 RPM;

Then the porcine pericardium was soaked in 30 mM 2-aminopent-4-enoic acid aqueous solution at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

The porcine pericardium was soaked in 30 mM 2-aminopent-4-enoic acid aqueous solution for 2 hours;

After cleaning, the porcine pericardium was soaked in 5 wt % 3-[N,N-dimethyl-[2-(2-methylprop-2-enoyloxy)ethyl]ammonium]propane-1-sulfonate aqueous solution at 37° C. for 12 hours to ensure sufficient physical permeation of 3-[N,N-dimethyl-[2-(2-methylprop-2-enoyloxy)ethyl]ammonium]propane-1-sulfonate;

An initiator of ammonium persulfate and sodium bisulfite was added to initiate the reaction for 24 hours at 37° C., wherein the molar concentrations of ammonium persulfate and sodium bisulfite are both 30 mM.

After the reaction is completed, the porcine pericardium was washed with distilled water, soaked in glycerol, and dehydrated to obtain a dry valve. The sample obtained in this example was recorded as sample 27.

### Example 28

Preparation of modified hyaluronic acid: 2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Preparation of modified polylysine: polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

In this example, freshly collected porcine pericardium was washed with distilled water for 2 hours at 4° C. with shaking at 100 RPM, and then soaked in 60 mM (calculated as lysine) modified polylysine aqueous solution at room temperature for 12 hours. Then, glutaraldehyde solution was added to a mass concentration of 250 mM. After reaction on a shaker at 37° C. for 24 hours, the pericardium material was taken out and cleaned, and soaked in a 50 mg/ml modified hyaluronic acid aqueous solution at room temperature for 12 hours, and then soaked in 2.5% ammonium persulfate and 0.25% N,N,N′,N′-tetramethylethylenediamine at 37° C. for 12 hours, and finally washed with distilled water, recorded as sample 28. Compared with control sample 2, the water contact angle of sample 28 decreased, the lactate dehydrogenase activity decreased, and the calcium ion content decreased, which can improve the hydrophilic properties, blood compatibility and anti-calcification ability of the biological material, potentially extending its lifespan.

### Alizarin Red Staining Experiment

The alizarin red staining experiment was conducted on sample 23, sample 24, sample 25, sample 26, sample 27 and control sample 6 using the same test method as the aforementioned alizarin red staining experiment.

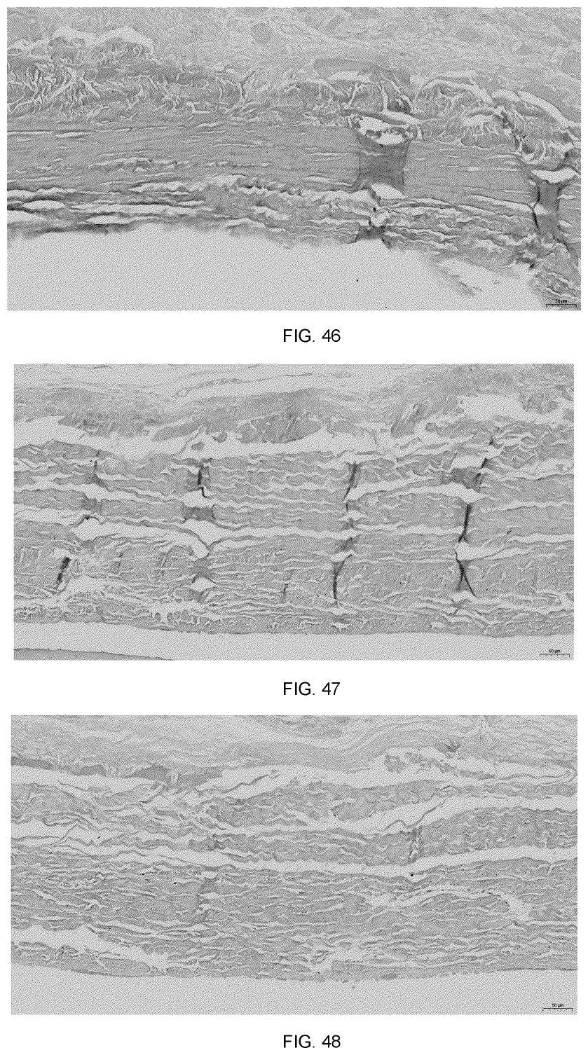

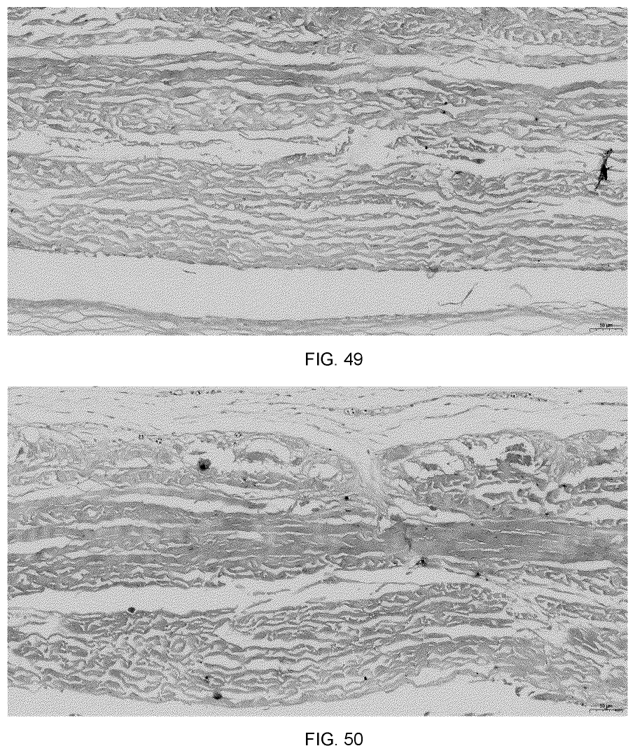

The control sample 6, sample 23, sample 24, sample 25, sample 26, and sample 27 after being implanted subcutaneously in rats for 30 days were stained through the alizarin red staining experiment to characterize the degree of calcification of samples. Images of the alizarin red staining results of the sample slices after being implanted subcutaneously in rats for 30 days are shown in FIGS. 45-50, wherein the darker the color of the sample after alizarin red staining, the higher the degree of calcification. Compared with the alizarin red staining result of slice of control sample 6 (FIG. 45), the color of the alizarin red staining results of sample 23, sample 24, sample 25, sample 26 and sample 27 is obviously lighter, which indicates that the degree of calcification of sample 23, sample 24, sample 25, sample 26 and sample 27 is lower than that of control sample 6, that is, compared with control sample 6, sample 23, sample 24, sample 25, sample 26 and sample 27 have certain anti-calcification effect. The alizarin red staining results of samples 23, 24, 25, 26, 27 and control group 6 after being implanted subcutaneously in rats for 30 days show that the method for preparing biological valve materials of the present disclosure can improve the anti-calcification performance of biological valves.

### Control Example 7

Freshly collected porcine pericardium was washed with distilled water for 2 hours at 4° C. with shaking at 100 RPM, then soaked in 100 mM glutaraldehyde solution and cross-linked for 24 hours at room temperature with shaking at 100 RPM to obtain control sample 7.

### Example 29

Freshly collected porcine pericardium was washed with distilled water for 2 hours at 4° C. with shaking at 100 RPM;

The porcine pericardium was soaked in 30 mM arginine aqueous solution at 37° C. for 2 hours;

Then glutaraldehyde was added to a final concentration of 150 mM, wherein the porcine pericardium was soaked for 24 hours at 37° C. with shaking at 100 RPM;

The porcine pericardium was taken out and washed with distilled water;

The porcine pericardium was soaked in 50 mM arginine aqueous solution at 37° C. for 12 hours, and then washed with distilled water to obtain a sample, which was recorded as sample 29.

### Example 30

Freshly collected porcine pericardium was washed with distilled water for 2 hours at 4° C. with shaking at 100 RPM;

The porcine pericardium was soaked in 30 mM arginine aqueous solution at 37° C. for 2 hours;

Then glutaraldehyde was added to a final concentration of 150 mM, wherein the porcine pericardium was soaked for 24 hours at 37° C. with shaking at 100 RPM;

The porcine pericardium was taken out and washed with distilled water;

The porcine pericardium was soaked in glycerol to obtain a dry valve, which was recorded as sample 30.

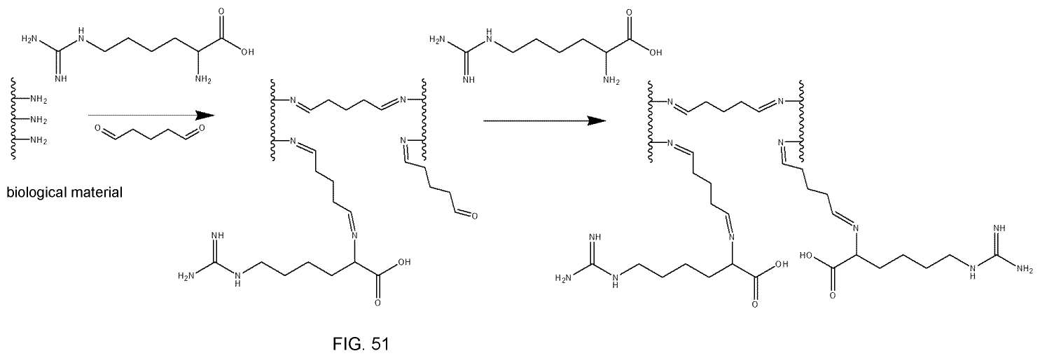

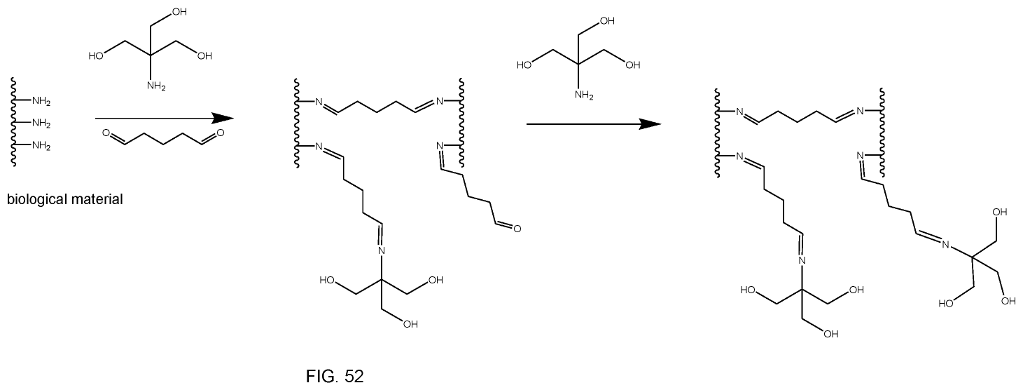

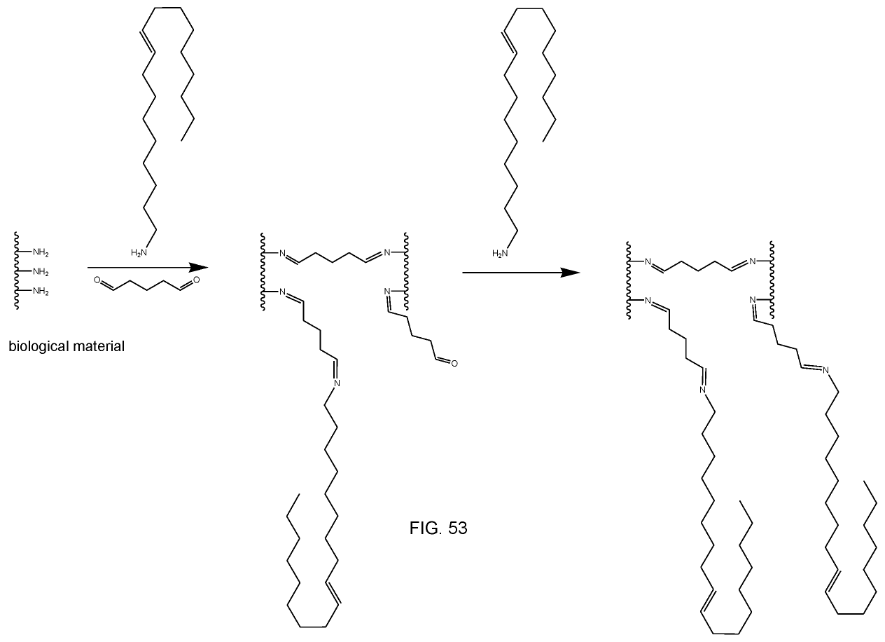

The reaction schematic diagram of Example 29 and Example 30 is shown in FIG. 51.

### Example 31

Freshly collected porcine pericardium was washed with distilled water for 2 hours at 4° C. with shaking at 100 RPM;

The porcine pericardium was soaked in 30 mM trihydroxymethyl aminomethane aqueous solution at 37° C. for 2 hours;

Then glutaraldehyde was added to a final concentration of 150 mM, wherein the porcine pericardium was soaked for 24 hours at 37° C. with shaking at 100 RPM;

The porcine pericardium was taken out and washed with distilled water;

The porcine pericardium was soaked in 50 mM trihydroxymethyl aminomethane aqueous solution at 37° C. for 12 hours, and then washed with distilled water to obtain a sample, which was recorded as sample 31.

The reaction schematic diagram of Example 31 is shown in FIG. 52.

### Example 32

Freshly collected porcine pericardium was washed with distilled water for 2 hours at 4° C. with shaking at 100 RPM;

The porcine pericardium was soaked in 20 mM oleylamine ethanol aqueous solution (50% ethanol, v/v) at 37° C. for 2 hours;

Then glutaraldehyde was added to a final concentration of 100 mM, wherein the porcine pericardium was soaked for 24 hours at 37° C. with shaking at 100 RPM;

The porcine pericardium was taken out and washed with distilled water;

Then, the porcine pericardium was soaked in 20 mM oleylamine ethanol aqueous solution (50% ethanol, v/v) at 37° C. for 12 hours; the sample was then washed with ethanol aqueous solution (50% ethanol, v/v) and recorded as sample 32.

The reaction schematic diagram of Example 32 is shown in FIG. 53.

### Example 33

Freshly collected porcine pericardium was washed with distilled water for 2 hours at 4° C. with shaking at 100 RPM;

The porcine pericardium was soaked in 20 mM dodecylamine ethanol aqueous solution (50% ethanol, v/v) for 2 hours at 37° C.;

Then glutaraldehyde was added to a final concentration of 100 mM, wherein the porcine pericardium was soaked for 24 hours at 37° C. with shaking at 100 RPM;

The porcine pericardium was taken out and washed with distilled water;

Then, the porcine pericardium was soaked in 20 mM dodecylamine ethanol aqueous solution (50% ethanol, v/v) at 37° C. for 12 hours; the sample was then washed with ethanol aqueous solution (50% ethanol, v/v) and recorded as sample 33.

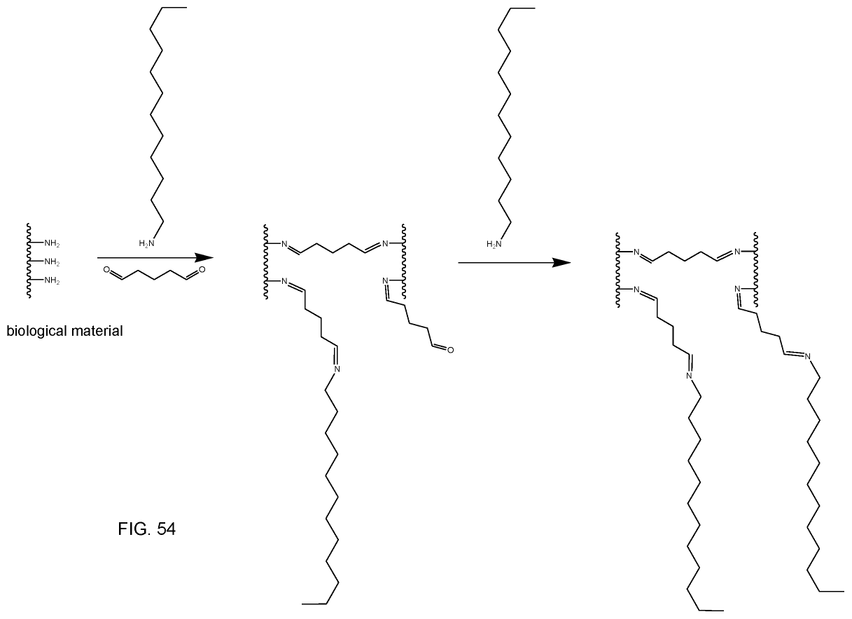

The reaction schematic diagram of Example 33 is shown in FIG. 54.

### Example 34

Freshly collected porcine pericardium was washed with distilled water for 2 hours at 4° C. with shaking at 100 RPM;

The porcine pericardium was soaked in 30 mM 2-amino-7-en-octanoic acid ethanol aqueous solution (40% ethanol, v/v) for 2 hours at 37° C.;

Then glutaraldehyde was added to a final concentration of 120 mM, wherein the porcine pericardium was soaked for 24 hours at 37° C. with shaking at 100 RPM;

The porcine pericardium was taken out and washed with distilled water;

Then, the porcine pericardium was soaked in 30 mM 2-amino-7-en-octanoic acid ethanol aqueous solution (40% ethanol, v/v) for 12 hours at 37° C.; the sample was then washed with ethanol aqueous solution (40% ethanol, v/v) and recorded as sample 34.

### Example 35

Freshly collected porcine pericardium was washed with distilled water for 2 hours at 4° C. with shaking at 100 RPM;

The porcine pericardium was soaked in 20 mM 4-penten-1-amine solution at 37° C. for 2 hours;

Then glutaraldehyde was added to a final concentration of 120 mM, wherein the porcine pericardium was soaked for 24 hours at 37° C. with shaking at 100 RPM;

The porcine pericardium was taken out and washed with distilled water;

Then, the porcine pericardium was soaked in a 20 mM 4-penten-1-amine solution at 37° C. for 12 hours; the sample was then washed with aqueous solution and recorded as sample 35.

### Example 36

Freshly collected porcine pericardium was washed with distilled water for 2 hours at 4° C. with shaking at 100 RPM;

The porcine pericardium was soaked in 3-butene-1-amine aqueous solution at 37° C. for 2 hours;

Then glutaraldehyde was added to a final concentration of 120 mM, wherein the porcine pericardium was soaked for 24 hours at 37° C. with shaking at 100 RPM;

The porcine pericardium was taken out and washed with distilled water;

Then, the porcine pericardium was soaked in 30 mM 3-butene-1-amine aqueous solution at 37° C. for 12 hours; the sample was then washed with distilled water and recorded as sample 36.

### Example 37

Freshly collected porcine pericardium was washed with distilled water for 2 hours at 4° C. with shaking at 100 RPM;

he porcine pericardium was soaked in 20 mM 4-penten-1-amine solution at 37° C. for 2 hours;

Then glutaraldehyde was added to a final concentration of 120 mM, wherein the porcine pericardium was soaked for 24 hours at 37° C. with shaking at 100 RPM;

The porcine pericardium was taken out and washed with distilled water;

Then, the porcine pericardium was soaked in an ethanol aqueous solution of oleylamine (50% ethanol, v/v) at 37° C. for 12 hours; the sample was then washed with distilled water and recorded as sample 37.

### Example 38

Freshly collected porcine pericardium was washed with distilled water for 2 hours at 4° C. with shaking at 100 RPM;

The porcine pericardium was soaked in 30 mM 2-amino-7-en-octanoic acid ethanol aqueous solution (40% ethanol, v/v) for 2 hours at 37° C.;

Then glutaraldehyde was added to a final concentration of 120 mM, wherein the porcine pericardium was soaked for 24 hours at 37° C. with shaking at 100 RPM;

The porcine pericardium was taken out and washed with distilled water;

Then, the porcine pericardium was soaked in an ethanol aqueous solution of oleylamine (50% ethanol, v/v) for 12 hours at 37° C.; the sample was then washed with an ethanol aqueous solution (50% ethanol, v/v) and recorded as sample 38.

### Example 39

Freshly collected porcine pericardium was washed with distilled water for 2 hours at 4° C. with shaking at 100 RPM;

The porcine pericardium was soaked in 30 mM 2-amino-7-en-octanoic acid ethanol aqueous solution (40% ethanol, v/v) for 2 hours at 37° C.;

Then glutaraldehyde was added to a final concentration of 120 mM, wherein the porcine pericardium was soaked for 24 hours at 37° C. with shaking at 100 RPM;

The porcine pericardium was taken out and washed with distilled water;

Then, the porcine pericardium was soaked in 30 mM 2-aminopent-4-enoic acid aqueous solution at 37° C. for 12 hours; the sample was then washed with distilled water and recorded as sample 39.

### Example 40

Freshly collected porcine pericardium was washed with distilled water for 2 hours at 4° C. with shaking at 100 RPM;

The porcine pericardium was soaked in 30 mM arginine solution at 37° C. for 2 hours;

Then glutaraldehyde was added to a final concentration of 120 mM, wherein the porcine pericardium was soaked for 24 hours at 37° C. with shaking at 100 RPM;

The porcine pericardium was taken out and washed with distilled water;

Then, the porcine pericardium was soaked in 50 mM trihydroxymethyl aminomethane aqueous solution at 37° C. for 12 hours; the sample was then washed with distilled water and recorded as sample 40.

### Example 41

Preparation of modified hyaluronic acid: 2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Preparation of modified polylysine: polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

In this example, freshly collected porcine pericardium was washed with distilled water for 2 hours at 4° C. with shaking at 100 RPM, and then soaked in 60 mM (calculated as lysine) modified polylysine aqueous solution at room temperature for 12 hours. Then, glutaraldehyde solution was added to a mass concentration of 250 mM. After reaction on a shaker at 37° C. for 24 hours, the pericardium material was taken out and cleaned, and soaked in a 50 mg/ml modified hyaluronic acid aqueous solution at room temperature for 12 hours, and then soaked in 2.5% ammonium persulfate and 0.25% N,N,N′,N′-tetramethylethylenediamine at 37° C. for 12 hours, and finally washed with distilled water, recorded as sample 41. Compared with control sample 2, the water contact angle of sample 41 decreased, the lactate dehydrogenase activity decreased, and the calcium ion content decreased, which can improve the hydrophilic properties, blood compatibility and anti-calcification ability of the biological material, potentially extending its lifespan.

### Blood Contact Experiment:

Blood contact experiment was conducted on control sample 7, sample 29 and sample 31 using the same test method as the aforementioned blood contact experiment.

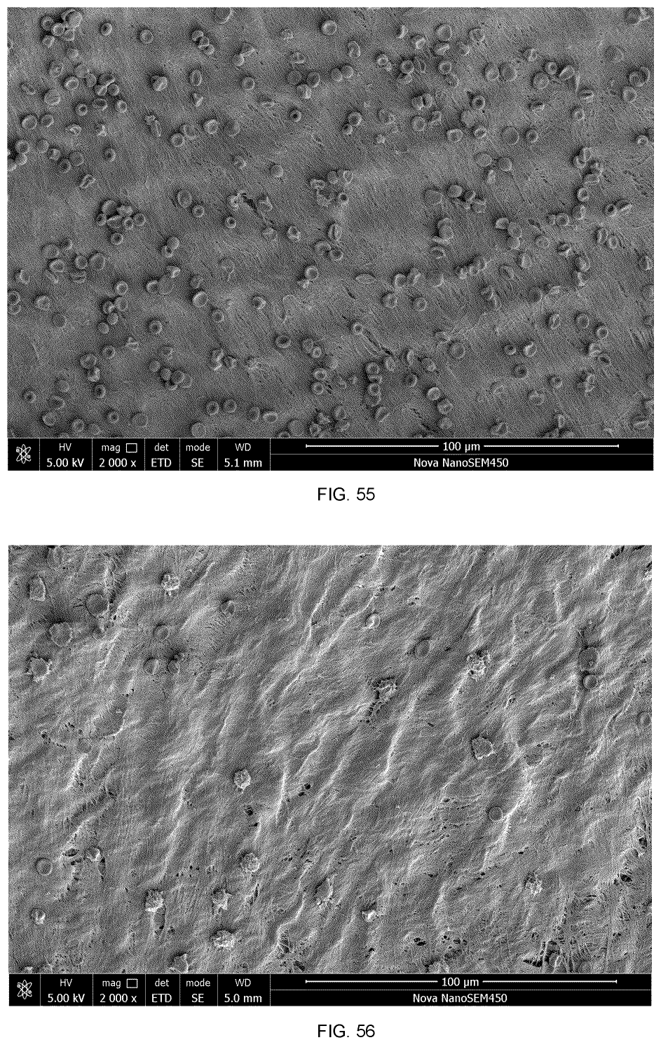

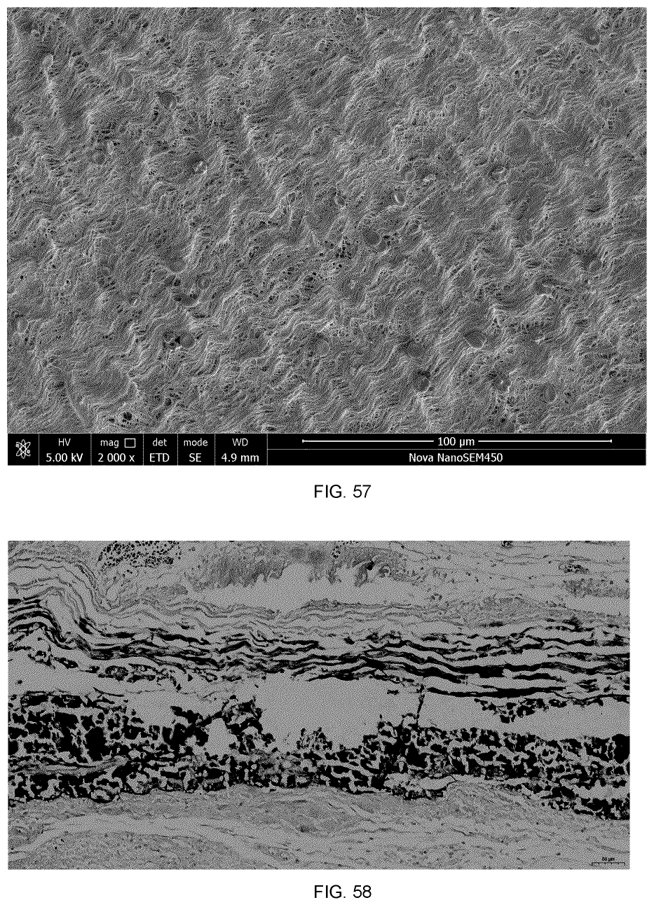

The scanning electron microscope images of the blood contact experiment of control sample 7, sample 29 and sample 31 are shown in FIG. 55, FIG. 56 and FIG. 57. After contact and incubation with rabbit blood, more adherent and aggregated blood cells were observed in the scanning electron microscope image of control sample 7, while samples 29 and 31 had fewer adherent blood cells, with only a few blood cells dispersedly adhered to the surface. The results show that sample 29 and sample 31 can inhibit the adhesion of blood cells to a certain extent, thereby reducing the risk of coagulation, and have anticoagulant effects. The blood contact results show that the method for preparing biological valve materials of the present disclosure can improve the anticoagulant performance of biological valves.

The blood contact experiment results of samples 35 to 40 show that they also have similar properties to samples 29 and 31, improving the anticoagulant performance of biological valve materials.

Alizarin red staining experiment after subcutaneous implantation in rats for 30 days:

The alizarin red staining experiment was conducted on sample 30, sample 32, sample 33, sample 34 and control sample 7 using the same test method as the aforementioned alizarin red staining experiment.

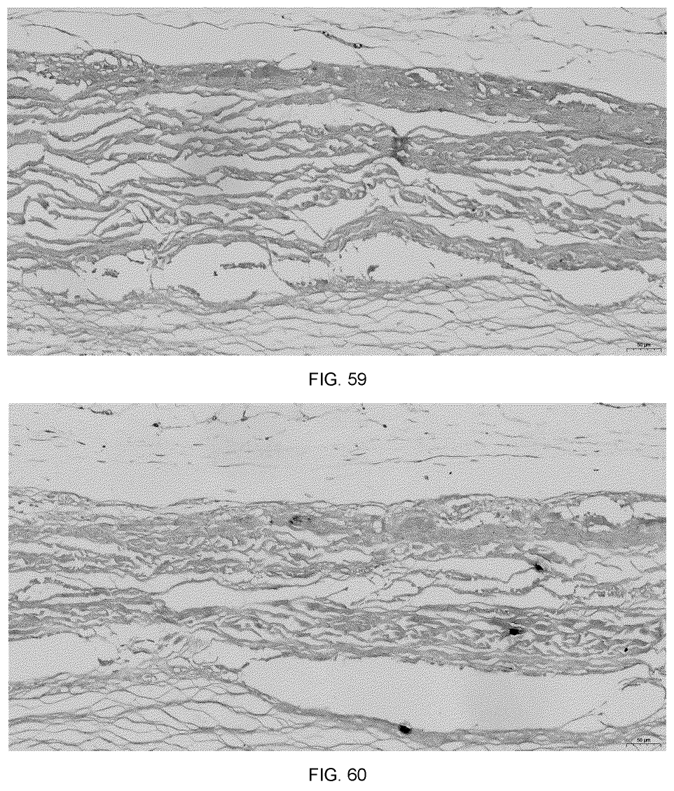

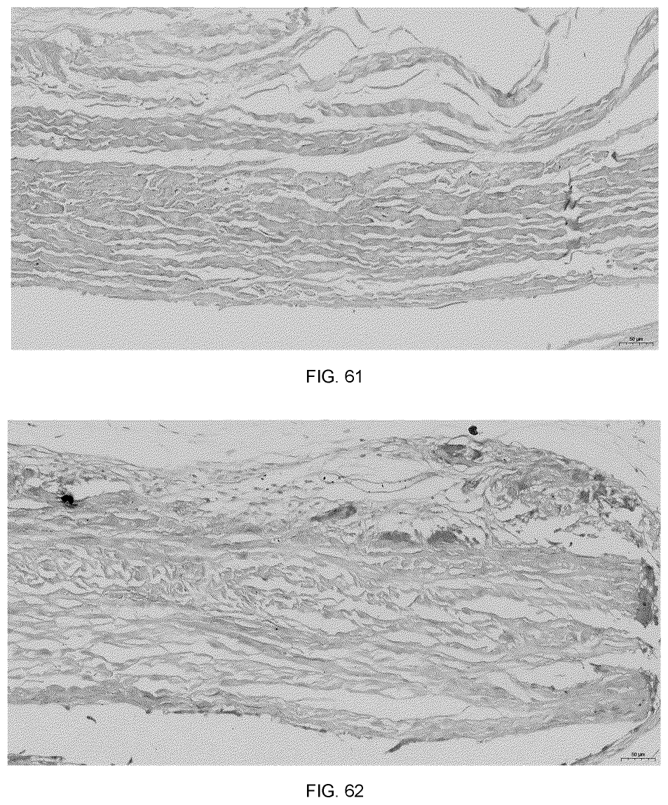

The control sample 7, sample 30, sample 32, sample 33, and sample 34 after being implanted subcutaneously in rats for 30 days were stained through the alizarin red staining experiment to characterize the degree of calcification of samples. Images of the alizarin red staining results of sample slices of control sample 7, sample 30, sample 32, sample 33, and sample 34 after being implanted subcutaneously in rats for 30 days are shown in FIG. 58 to 62, wherein the darker the color of the sample after alizarin red staining, the higher the degree of calcification. Compared with the alizarin red staining result of slice of control sample 7 (FIG. 58), the color of the alizarin red staining results of sample 30, sample 32, sample 33, and sample 34 is obviously lighter, which indicates that the degree of calcification of sample 30, sample 32, sample 33 and sample 34 is lower than that of control sample 7, that is, compared with control sample 7, sample 30, sample 32, sample 33, and sample 34 have certain anti-calcification effect. The alizarin red staining results of sample 30, sample 32, sample 33, sample 34 and control sample 7 after being implanted subcutaneously in rats for 30 days show that the method for preparing biological valve materials of the present disclosure can improve the anti-calcification performance of biological valves.

The alizarin red staining experiment results of samples 35 to 40 show that they also have similar properties to samples 29 and 32, improving the anti-calcification performance of biological valve materials.

### Control Group 8

Freshly collected porcine pericardium was washed with distilled water for 2 hours at 4° C. with shaking at 100 RPM, then soaked in 0.30% (w/w) glutaraldehyde solution and cross-linked for 48 hours at room temperature with shaking at 100 RPM to obtain control sample 8.

### Example 42

In this example, freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in a propanol aqueous solution of 5% (v/v) glycidyl methacrylate at room temperature for double bonding modification of the glutaraldehyde cross-linked porcine pericardium for 72 hours, wherein the solvent of the double bonding solution was 20% (v/v) propanol aqueous solution.

After the double bonding modification, the double-bonded glutaraldehyde cross-linked porcine pericardium was washed with deionized water, and then soaked in a mixed solution of 20 mM potassium persulfate and 10 mM sodium bisulfite to initiate polymerization of double bonds on the double-bonded glutaraldehyde cross-linked porcine pericardium for 8 hours at 37° C., thereby obtaining double-bond post-crosslinked porcine pericardium, which was recorded as sample 42.

### Example 43

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an isopropanol aqueous solution of 6% (v/v) glycidyl acrylate at room temperature for double bonding modification of the glutaraldehyde cross-linked porcine pericardium for 72 hours, wherein the solvent of the double bonding solution was 20% (v/v) isopropanol aqueous solution.

After the double bonding modification, the double-bonded glutaraldehyde cross-linked porcine pericardium was washed with deionized water, and then soaked in a mixed solution of 20 mM ammonium persulfate and 5 mM sodium bisulfite to initiate polymerization of double bonds on the double-bonded glutaraldehyde cross-linked porcine pericardium for 8 hours at 37° C., thereby obtaining double-bond post-crosslinked porcine pericardium, which was recorded as sample 43.

### Example 44

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an isopropanol aqueous solution containing both 4% (v/v) glycidyl acrylate and 4% (v/v) allyl glycidyl ether at room temperature for double bonding modification of the glutaraldehyde cross-linked porcine pericardium for 72 hours, wherein the solvent of the double bonding solution was 30% (v/v) ethanol aqueous solution.

After the double bonding modification, the double-bonded glutaraldehyde cross-linked porcine pericardium was washed with deionized water, and then soaked in a mixed solution of 20 mM ammonium persulfate and 10 mM sodium bisulfite to initiate polymerization of double bonds on the double-bonded glutaraldehyde cross-linked porcine pericardium for 7 hours at 37° C., thereby obtaining double-bond post-crosslinked porcine pericardium, which was recorded as sample 44, numbered as GAGA-PP-3.

### Example 45

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an isopropanol aqueous solution of 5% (v/v) glycidyl methacrylate and 2% (v/v) glycidyl acrylate at room temperature for double bonding modification of the glutaraldehyde cross-linked porcine pericardium for 72 hours, wherein the solvent of the double bonding solution was 35% (v/v) isopropanol aqueous solution.

After the double bonding modification, the double-bonded glutaraldehyde cross-linked porcine pericardium was washed with deionized water, and then soaked in a mixed solution of 20 mM sodium persulfate and 5 mM sodium bisulfite to initiate polymerization of double bonds on the double-bonded glutaraldehyde cross-linked porcine pericardium for 8 hours at 37° C., thereby obtaining double-bond post-crosslinked porcine pericardium, which was recorded as sample 45.

### Example 46

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an ethanol aqueous solution of 4% (v/v) glycidyl methacrylate at room temperature for double bonding modification of the glutaraldehyde cross-linked porcine pericardium for 72 hours, wherein the solvent of the double bonding solution was 20% (v/v) ethanol aqueous solution.

After the double bonding modification, an initiator of ammonium persulfate and sodium bisulfite was added to initiate polymerization of double bonds on the double-bonded glutaraldehyde cross-linked porcine pericardium for 8 hours at 37° C., thereby obtaining double-bond post-crosslinked porcine pericardium, which was recorded as sample 46, wherein the concentration of ammonium persulfate was 20 mM and the concentration of sodium bisulfite was 5 mM.

### Example 47

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an isobutanol aqueous solution of 4% (v/v) glycidyl methacrylate at room temperature for double bonding modification of the glutaraldehyde cross-linked porcine pericardium for 72 hours, wherein the solvent of the double bonding solution was 15% (v/v) isobutanol aqueous solution.

After the double bonding modification, the double-bonded glutaraldehyde cross-linked porcine pericardium was washed with deionized water, and then soaked in a mixed solution of 20 mM ammonium persulfate and 5 mM sodium bisulfite to initiate polymerization of double bonds on the double-bonded glutaraldehyde cross-linked porcine pericardium for 8 hours at 37° C., thereby obtaining double-bond post-crosslinked porcine pericardium, which was recorded as sample 47.

### Example 48

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an isopropanol aqueous solution of 4% (v/v) glycidyl acrylate at room temperature for double bonding modification of the glutaraldehyde cross-linked porcine pericardium for 48 hours, wherein the solvent of the double bonding solution was 20% (v/v) methanol aqueous solution.

After the double bonding modification, the double-bonded glutaraldehyde cross-linked porcine pericardium was washed with deionized water, and then soaked in a mixed solution of 20 mM ammonium persulfate and 6.5 mM sodium sulfite to initiate polymerization of double bonds on the double-bonded glutaraldehyde cross-linked porcine pericardium for 10 hours at 37° C., thereby obtaining double-bond post-crosslinked porcine pericardium, which was recorded as sample 48.

### Example 49

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an ethylene glycol aqueous solution of 4% (v/v) glycidyl methacrylate at room temperature for double bonding modification of the glutaraldehyde cross-linked porcine pericardium for 72 hours, wherein the solvent of the double bonding solution was 20% (v/v) ethylene glycol aqueous solution.

After the double bonding modification, the double-bonded glutaraldehyde cross-linked porcine pericardium was washed with deionized water, and then soaked in a mixed solution of 40 mM ammonium persulfate and 15 mM sodium bisulfite to initiate polymerization of double bonds on the double-bonded glutaraldehyde cross-linked porcine pericardium for 7 hours at 37° C., thereby obtaining double-bond post-crosslinked porcine pericardium, which was recorded as sample 49.

### Example 50

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in a propanol aqueous solution of 7% (v/v) glycidyl acrylate at room temperature for double bonding modification of the glutaraldehyde cross-linked porcine pericardium for 60 hours, wherein the solvent of the double bonding solution was 40% (v/v) propanol aqueous solution.

After the double bonding modification, the double-bonded glutaraldehyde cross-linked porcine pericardium was washed with deionized water, and then soaked in a mixed solution of 30 mM sodium persulfate and 10 mM sodium bisulfite to initiate polymerization of double bonds on the double-bonded glutaraldehyde cross-linked porcine pericardium for 8 hours at 37° C., thereby obtaining double-bond post-crosslinked porcine pericardium, which was recorded as sample 50.

### Example 51

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

After cleaning with deionized water, the porcine pericardium was soaked in an isopropanol aqueous solution containing 6% (v/v) glycidyl methacrylate and 3% (v/v) glycidyl acrylate at room temperature for double bonding modification of the glutaraldehyde cross-linked porcine pericardium for 84 hours, wherein the solvent of the double bonding solution was 50% (v/v) ethanol aqueous solution.

After the double bonding modification, the double-bonded glutaraldehyde cross-linked porcine pericardium was washed with deionized water, and then soaked in a mixed solution of 40 mM ammonium persulfate and 10 mM sodium sulfite to initiate polymerization of double bonds on the double-bonded glutaraldehyde cross-linked porcine pericardium for 12 hours at 37° C., thereby obtaining double-bond post-crosslinked porcine pericardium, which was recorded as sample 51.

### Performance Characterization of the Samples of Examples 42 to 51 and Control Group 8:

In order to characterize the change in cross-linking degree of glutaraldehyde cross-linked biological valve materials before and after double-bond post-crosslinking treatment, the thermal stability and cross-linking degree of biological valve materials were characterized by measuring the thermal shrinkage temperature of biological valve materials; the stability of biological valve materials were characterized through enzyme degradation experiment; the degree of calcification (anti-calcification performance) of the samples were characterized through rat subcutaneous implantation experiment.

### Thermal Shrinkage Temperature Measurement:

The biological valve material was cut into a circular sheet with a diameter of 0.6 cm, which was placed in a crucible after drying, and then the thermal shrinkage temperature of the biological valve material was measured on a differential scanning calorimeter in the range of 40-120° C. at a heating rate of 10° C./min. Thermal stability and cross-linking degree of the biological valve material were characterized by measuring the thermal shrinkage temperature. The higher the thermal shrinkage temperature, the higher the thermal stability and cross-linking degree.

TABLE 10
Thermal shrinkage temperature of samples

| | Thermal shrinkage |
|---|---|
| Sample | temperature (° C.) |
| Control group 8 (glutaraldehyde cross- | 84.7 |
| linked porcine pericardium) | |
| Example 42 | 88.9 |
| Example 43 | 89.3 |
| Example 50 | 91.5 |
| Example 51 | 92.0 |

Through the thermal shrinkage temperature measurement of Example 42, Example 43, Example 50, Example 51 and the control group 8 (glutaraldehyde cross-linked porcine pericardium), it can be seen that, as shown in Table 10, the thermal shrinkage temperatures of Example 42, Example 43, Example 50 and Example 51 are all higher than that of control group 8 (glutaraldehyde cross-linked porcine pericardium), that is, the thermal stability and cross-linking degree of Example 42, Example 43, Example 50 and Example 51 were all higher than those in the control group 8 (glutaraldehyde cross-linked porcine pericardium). The experimental results of the thermal shrinkage temperature measurement show that the double-bond post-crosslinking method for preparing biological valve materials of the present disclosure can improve the thermal stability and cross-linking degree of biological valves.

### Enzyme Degradation Experiment

The collagenase degradation weight loss rates of sample 44, sample 47, sample 45, sample 51 and control group 8 were measured using the same method as the aforementioned enzyme degradation experiment.

TABLE 11
Enzymatic degradation weight loss rate of samples

| | Enzymatic degradation |
|---|---|
| Sample | weight loss rate (%) |
| Control group 8 (glutaraldehyde cross- | 7.45 ± 1.33 |
| linked porcine pericardium) | |
| Sample 44 | 5.31 ± 0.30 |
| Sample 45 | 4.47 ± 1.05 |
| Sample 47 | 5.12 ± 0.97 |
| Sample 51 | 3.06 ± 0.59 |

The enzyme degradation experiment was conducted on control group 8 (glutaraldehyde cross-linked porcine pericardium), sample 44, sample 45, sample 47, and sample 51 to characterize the cross-linking efficiency of samples, and the enzymatic degradation weight loss rates of samples after control group 8 (glutaraldehyde cross-linked porcine pericardium), sample 44, sample 45, sample 47 and sample 51 were treated with collagenase I were calculated as shown in Table 11. The enzymatic degradation weight loss rates of samples 44, 45, 47, and 51 were all lower than that of the control group (glutaraldehyde cross-linked porcine pericardium), which indicates that samples 44, 45, 47, and 51 have higher stability than the control group (glutaraldehyde cross-linked porcine pericardium). The results of the enzyme degradation experiment show that the double-bond post-crosslinking method for preparing biological valve materials of the present disclosure can improve the stability of biological valves.

### Anti-Calcification Test

The biological valve materials were cut into 0.8*0.8 cm² sheets, sterilized and implanted subcutaneously in rats for 30 days before being taken out. Each sample was divided into two parts. One part was decapsulated and freeze-dried and weighed, and then digested with 6 M hydrochloric acid to measure the calcium content per gram of sample. The other part of the sample was fixed with paraformaldehyde tissue fixative. After fixation, the sampled were taken out and smoothed with a scalpel, and then transferred to a dehydration box. The material samples were dehydrated using gradient ethanol. After dehydration, the material samples were transferred to an embedding machine and embedded in melted wax, and then transferred to a −20° C. refrigerator to cool and trimmed. Slices with 5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner

TABLE 12
Calcium content of samples after subcutaneous implantation
in rats for 30 days

| Sample | Calcium content (mg/g) |
|---|---|
| Control group 8 (glutaraldehyde cross- | 74.9 ± 12.3 |
| linked porcine pericardium) | |
| Sample 42 | 15.1 ± 4.7 |
| Sample 46 | 8.4 ± 4.6 |
| Sample 48 | 12.7 ± 5.1 |

The calcium contents of sample 42, sample 46, sample 48 and control group 8 (glutaraldehyde cross-linked porcine pericardium) after being implanted subcutaneously in rats for 30 days was measured to characterize the degree of calcification of samples. As shown in Table 12, the calcium contents of sample 42, sample 46, and sample 48 after being implanted subcutaneously in rats for 30 days were all lower than that of the control group (glutaraldehyde cross-linked porcine pericardium). This result indicates that the double-bond post-crosslinking method for preparing biological valve materials can improve the anti-calcification performance of biological valves.

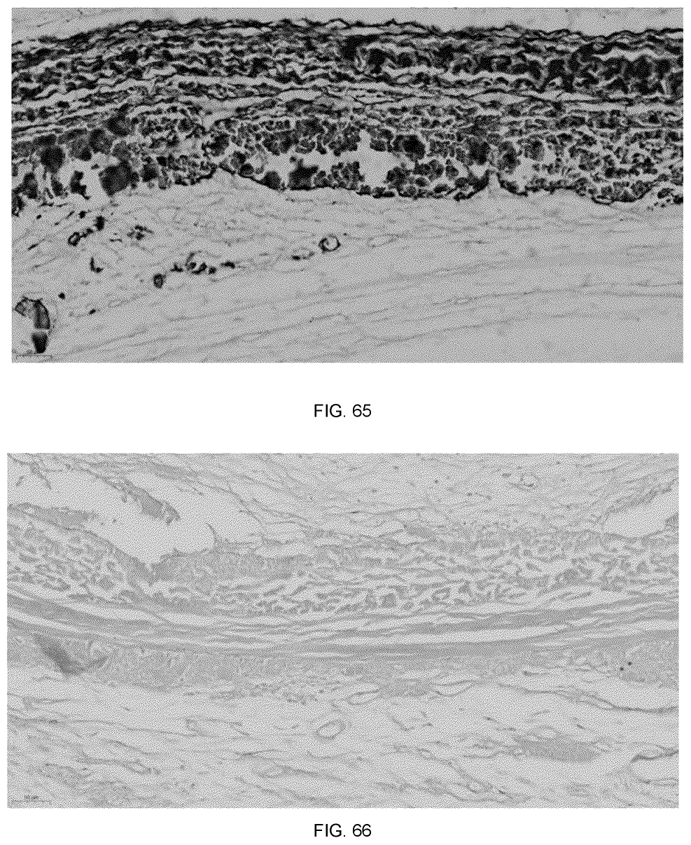

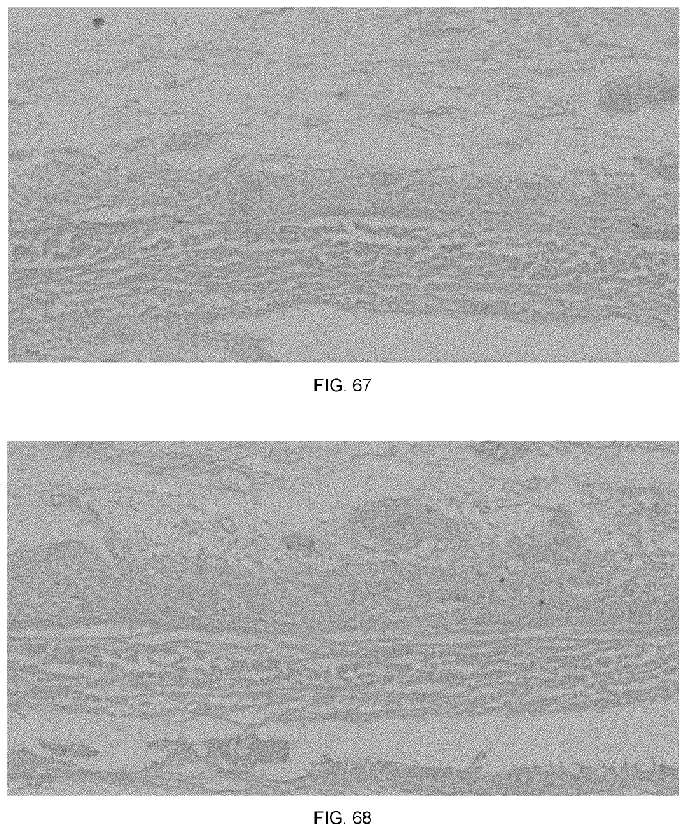

The alizarin red staining experiment was conducted on control group 8 (glutaraldehyde cross-linked porcine pericardium), sample 42, sample 46, and sample 48 after being implanted subcutaneously in rats for 30 days to directly observe the degree of calcification of samples. Images of the alizarin red staining results of sample slices after being implanted subcutaneously in rats for 30 days are shown in FIGS. 65 to 68, wherein the darker the color of the sample after alizarin red staining, the higher the degree of calcification. Compared with the alizarin red staining result of slice of control sample 8 (glutaraldehyde cross-linked porcine pericardium) (FIG. 65), the color of the alizarin red staining results of slices of sample 42 (FIG. 66), sample 46 (FIG. 67), and sample 48 (FIG. 68) is obviously lighter, which directly indicates that the degree of calcification of sample 42, sample 46, and sample 48 is lower than that of the control group 8, that is, compared with control group 8, sample 42, sample 46, and sample 48 have stronger anti-calcification effect. The alizarin red staining results of biological valve materials after being implanted subcutaneously in rats for 30 days show that the double-bond post-crosslinking method for preparing biological valve materials of the present disclosure can improve the anti-calcification performance of biological valves.

### Control Example 9

Meanwhile, the simple glutaraldehyde cross-linked group was set as the control group, wherein the porcine pericardium was soaked in 0.625% (w/w) glutaraldehyde for 72 hours at room temperature to prepare glutaraldehyde cross-linked porcine pericardium, which was recorded as control sample 9.

### Example 52

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 5% (v/v) glycidyl methacrylate at room temperature for double bonding modification of the glutaraldehyde cross-linked porcine pericardium for 72 hours, wherein the solvent of the double bonding solution was 18% (v/v) isopropanol aqueous solution.

After the double bonding modification, the double-bonded glutaraldehyde cross-linked porcine pericardium was washed with deionized water, and then soaked in 2% (v/v) polyethylene glycol diacrylate aqueous solution for 2 hours.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the polyethylene glycol diacrylate for 8 hours at 37° C., thereby obtaining double bond copolymerized and post-crosslinked porcine pericardium, which was recorded as sample 52.

### Example 53

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in a propanol aqueous solution of 6% (v/v) glycidyl acrylate at room temperature for double bonding modification of the glutaraldehyde cross-linked porcine pericardium for 72 hours, wherein the solvent of the double bonding solution was 20% (v/v) propanol aqueous solution.

After the double bonding modification, the double-bonded glutaraldehyde cross-linked porcine pericardium was washed with deionized water, and then soaked in 2.5% (v/v) N-methyl-2-acrylamide solution for 1 hour;

An initiator was added to the above solution, which includes 20 mM potassium persulfate and 10 mM sodium sulfite, to further initiate polymerization between double bonds on the double-bonded glutaraldehyde cross-linked biological valve material and double bonds of the N-methyl-2-acrylamide for 8 hours at 37° C., thereby obtaining double bond copolymerized and post-crosslinked porcine pericardium, which was recorded as sample 53.

### Example 54

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution containing both 2% (v/v) glycidyl acrylate and 4% (v/v) allyl glycidyl ether at room temperature for double bonding modification of the glutaraldehyde cross-linked porcine pericardium for 72 hours, wherein the solvent of the double bonding solution was 30% (v/v) ethanol aqueous solution.

After the double bonding modification, the double-bonded glutaraldehyde cross-linked porcine pericardium was washed with deionized water, and then soaked in 2.5% (v/v) (ethane-1,2-diylbis(oxy))bis(ethane-2,1-diyl)diacrylate solution for 1 hour;

An initiator was added to the above solution, which includes 20 mM potassium persulfate and 10 mM sodium sulfite, to further initiate polymerization between double bonds on the double-bonded glutaraldehyde cross-linked biological valve material and double bonds of the (ethane-1,2-diylbis(oxy))bis(ethane-2,1-diyl)diacrylate for 8 hours at 37° C., thereby obtaining double bond copolymerized and post-crosslinked porcine pericardium, which was recorded as sample 54.

### Example 55

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 3% (v/v) glycidyl methacrylate and 2% (v/v) glycidyl acrylate at room temperature for double bonding modification of the glutaraldehyde cross-linked porcine pericardium for 48 hours, wherein the solvent of the double bonding solution was 25% (v/v) isopropanol aqueous solution.

After the double bonding modification, the double-bonded glutaraldehyde cross-linked porcine pericardium was washed with deionized water, and then soaked in 1.5% (v/v) ethane-1,2-diyl diacrylate solution for 1 hour.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the ethane-1,2-diyl diacrylate for 7 hours at 37° C., thereby obtaining double bond copolymerized and post-crosslinked porcine pericardium, which was recorded as sample 55.

### Example 56

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an ethanol aqueous solution of 4% (v/v) glycidyl methacrylate at room temperature for double bonding modification of the glutaraldehyde cross-linked porcine pericardium for 72 hours, wherein the solvent of the double bonding solution was 20% (v/v) ethanol aqueous solution.

After the double bonding modification, the double-bonded glutaraldehyde cross-linked porcine pericardium was washed with deionized water, and then soaked in 1.4% (v/v) N,N′-dimethylacrylamide solution for 1 hour.

An initiator was added to the above solution, which includes 20 mM sodium persulfate and 7 mM sodium bisulfite, to further initiate polymerization between double bonds on the double-bonded glutaraldehyde cross-linked biological valve material and double bonds of the N,N′-dimethylacrylamide for 7 hours at 37° C., thereby obtaining double bond copolymerized and post-crosslinked porcine pericardium, which was recorded as sample 56.

### Example 57

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isobutanol aqueous solution of 4% (v/v) glycidyl methacrylate at room temperature for double bonding modification of the glutaraldehyde cross-linked porcine pericardium for 72 hours, wherein the solvent of the double bonding solution was 15% (v/v) isobutanol aqueous solution.

After the double bonding modification, the double-bonded glutaraldehyde cross-linked porcine pericardium was washed with deionized water, and then soaked in 1.4% (v/v) N,N′-dimethylmethacrylamide solution for 5 hour.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the N,N′-dimethylmethacrylamide for 12 hours at 37° C., thereby obtaining double bond copolymerized and post-crosslinked porcine pericardium, which was recorded as sample 57.

### Example 58

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 4% (v/v) glycidyl acrylate at room temperature for double bonding modification of the glutaraldehyde cross-linked porcine pericardium for 48 hours, wherein the solvent of the double bonding solution was 20% (v/v) methanol aqueous solution.

After the double bonding modification, the double-bonded glutaraldehyde cross-linked porcine pericardium was washed with deionized water, and then soaked in a solution containing 1.0% (v/v) N,N′-dimethylacrylamide and 0.5% (v/v) N,N′-dimethylmethacrylamide for 1 hour;

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the N,N′-dimethylacrylamide and N,N′-dimethylmethacrylamide for 8 hours at 37° C., thereby obtaining double bond copolymerized and post-crosslinked porcine pericardium, which was recorded as sample 58.

### Example 59

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an ethylene glycol aqueous solution of 5% (v/v) glycidyl methacrylate at room temperature for double bonding modification of the glutaraldehyde cross-linked porcine pericardium for 72 hours, wherein the solvent of the double bonding solution was 25% (v/v) ethylene glycol aqueous solution.

After the double bonding modification, the double-bonded glutaraldehyde cross-linked porcine pericardium was washed with deionized water, and then soaked in 1.25% (v/v) N,N′-dimethylmethacrylamide solution for 5 hours;

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the N,N′-dimethylmethacrylamide for 12 hours at 37° C., thereby obtaining double bond copolymerized and post-crosslinked porcine pericardium, which was recorded as sample 59.

### Example 60

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in a propanol aqueous solution of 7% (v/v) glycidyl acrylate at room temperature for double bonding modification of the glutaraldehyde cross-linked porcine pericardium for 60 hours, wherein the solvent of the double bonding solution was 30% (v/v) propanol aqueous solution.

After the double bonding modification, the double-bonded glutaraldehyde cross-linked porcine pericardium was washed with deionized water, and then soaked in 1.0% (v/v) N-ethylacrylamide solution for 5 hours;

An initiator was added to the above solution, which includes 20 mM ammonium persulfate and 1 mM tetramethylethylenediamine, to further initiate polymerization between double bonds on the double-bonded glutaraldehyde cross-linked biological valve material and double bonds of the N,N′-dimethylmethacrylamide for 12 hours at 37° C., thereby obtaining double bond copolymerized and post-crosslinked porcine pericardium, which was recorded as sample 60.

### Example 61

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution containing 4% (v/v) glycidyl methacrylate and 2% (v/v) glycidyl acrylate at room temperature for double bonding modification of the glutaraldehyde cross-linked porcine pericardium for 84 hours, wherein the solvent of the double bonding solution was 25% (v/v) ethanol aqueous solution.

After the double bonding modification, the double-bonded glutaraldehyde cross-linked porcine pericardium was washed with deionized water, and then soaked in 1.50% (v/v) N,N′-dimethylmethacrylamide solution for 3 hours;

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the N,N′-dimethylmethacrylamide for 7 hours at 37° C., thereby obtaining double bond copolymerized and post-crosslinked porcine pericardium, which was recorded as sample 61.

### Performance Characterization of the Samples of Examples 52 to 61 and the Sample of Control Example 9:

In order to characterize the change in the cross-linking degree of glutaraldehyde cross-linked biological valve materials before and after double-bond post-copolymerization and cross-linking treatment, the thermal stability and cross-linking degree of biological valve materials were characterized by measuring the thermal shrinkage temperature of biological valve materials; the stability of biological valve materials were characterized through enzyme degradation experiment; the degree of calcification (anti-calcification performance) of the samples were characterized through rat subcutaneous implantation experiment; and the elasticity of biological valve materials were characterized by measuring their elastic angles.

### Thermal Shrinkage Temperature Measurement:

The biological valve material was cut into a circular sheet with a diameter of 0.6 cm, which was placed in a crucible after drying, and then the thermal shrinkage temperature of the biological valve material was measured on a differential scanning calorimeter in the range of 40-120° C. at a heating rate of 10° C./min. Thermal stability and cross-linking degree of the biological valve material were characterized by measuring the thermal shrinkage temperature. The higher the thermal shrinkage temperature, the higher the thermal stability and cross-linking degree.

TABLE 13
Thermal shrinkage temperature of biological valve materials

| Sample name | Thermal shrinkage temperature (° C.) |
|---|---|
| Control group 9 (glutaraldehyde cross- | 85.2 |
| linked porcine pericardium) | |
| Sample 52 | 91.3 |
| Sample 53 | 92.8 |
| Sample 54 | 90.7 |
| Sample 56 | 91.4 |
| Sample 561 | 90.1 |

Through the thermal shrinkage temperature measurement of control group 9 (glutaraldehyde cross-linked porcine pericardium), sample 52, sample 53, sample 54, sample 56, and sample 61, it can be seen that, as shown in Table 13, the thermal shrinkage temperatures of sample 52, sample 53, sample 54, sample 56 and sample 61 are all higher than that of control group 9 (glutaraldehyde cross-linked porcine pericardium), that is, the thermal stability and cross-linking degree of samples 52, 53, 54, 56 and 61 are all higher than those in the control group (glutaraldehyde cross-linked porcine pericardium). The experimental results of the thermal shrinkage temperature measurement show that the double-bond post-copolymerization and cross-linking method for preparing biological valve materials of the present disclosure can improve the thermal stability and cross-linking degree of biological valves.

### Elasticity Test Experiment

The biological valve material with uniform thickness was cut into a rectangular sample of 1*4.6 cm², which was clamped horizontally along the midline of the long side thereof. The angle at which the sample sag relative to the horizontal plane of the midline was tested to characterize the elasticity of the sample. The smaller the angle, the higher the elasticity.

TABLE 14
Elastic angle of biological valve materials

| Sample name | Elastic angle (°) |
|---|---|
| Control group 9 (glutaraldehyde cross- | 65 |
| linked porcine pericardium) | |
| Sample 52 | 35 |
| Sample 53 | 45 |
| Sample 54 | 56 |
| Sample 56 | 47 |
| Sample 61 | 50 |

The elasticity test experiment was conducted on sample 52, sample 53, sample 54, sample 56, sample 61 and control group 9 (glutaraldehyde cross-linked porcine pericardium) to characterize their elasticity. The elasticity test results are shown in Table 14. Compared with control group 9 (glutaraldehyde cross-linked porcine pericardium), the elastic angles of sample 52, sample 53, sample 54, sample 56 and sample 61 are lower, indicating that their elasticity is significantly improved than the control group (glutaraldehyde cross-linked porcine pericardium). The double-bond post-copolymerization and cross-linking method for preparing biological valve materials can improve the elasticity of biological valve materials, which is conducive to rapid recovery of shape after transcatheter implantation.

### Enzyme Degradation Experiment

The collagenase degradation weight loss rates of sample 52, sample 53, sample 56, sample 61 and control group 9 were measured using the same method as the aforementioned enzyme degradation experiment. The results are shown in Table 15.

TABLE 15
Enzymatic degradation weight loss rate of biological valve materials

| | Enzymatic degradation |
|---|---|
| Sample name | weight loss rate (%) |
| Control group (glutaraldehyde cross-linked | 7.27 ± 1.18 |
| porcine pericardium) | |
| Example 52 | 1.12 ± 0.40 |
| Example 53 | 3.61 ± 0.22 |
| Example 56 | 2.90 ± 0.45 |
| Example 61 | 3.27 ± 0.55 |

The enzyme degradation experiment was conducted on samples 52, 53, 56, 61 and control group 9 (glutaraldehyde cross-linked porcine pericardium) to characterize the cross-linking efficiency of samples, and the enzymatic degradation weight loss rates of samples after samples 52, sample 53, sample 56, sample 61 and control group 9 (glutaraldehyde cross-linked porcine pericardium) were treated with collagenase I were calculated as shown in Table 15. The enzymatic degradation weight loss rates of samples 52, sample 53, sample 56 and sample 61 were all lower than that of the control group 9 (glutaraldehyde cross-linked porcine pericardium), which indicates that samples 52, sample 53, sample 56 and sample 61 have higher stability than the control group (glutaraldehyde cross-linked porcine pericardium). The results of the enzyme degradation experiment show that the double-bond post-copolymerization and cross-linking method for preparing biological valve materials of the present disclosure can improve the stability of biological valves.

### Anti-Calcification Test

The biological valve materials of sample 52, sample 53, sample 56 and control group 9 (glutaraldehyde cross-linked porcine pericardium) were cut into 1*1 cm² sheets, and the anti-calcification test was conducted using the same method as the aforementioned anti-calcification test.

TABLE 16
Calcium content of biological valve materials after
subcutaneous implantation in rats for 30 days

| Sample name | Calcium content (mg/g) |
|---|---|
| Control group 9 (glutaraldehyde cross- | 67.3 ± 10.5 |
| linked porcine pericardium) | |
| Sample 52 | 5.4 ± 2.7 |
| Sample 53 | 8.1 ± 3.6 |
| Sample 56 | 13.9 ± 4.7 |

The calcium content of sample 52, sample 53, sample 56 and control group 9 (glutaraldehyde cross-linked porcine pericardium) after being implanted subcutaneously in rats for 30 days was measured to characterize the degree of calcification of samples. As shown in Table 16, the calcium contents of sample 52, sample 53, and sample 56 after being implanted subcutaneously in rats for 30 days were all lower than that of the control group 9 (glutaraldehyde cross-linked porcine pericardium). This result indicates that the double-bond post-copolymerization and cross-linking method for preparing biological valve materials can improve the anti-calcification performance of biological valves.

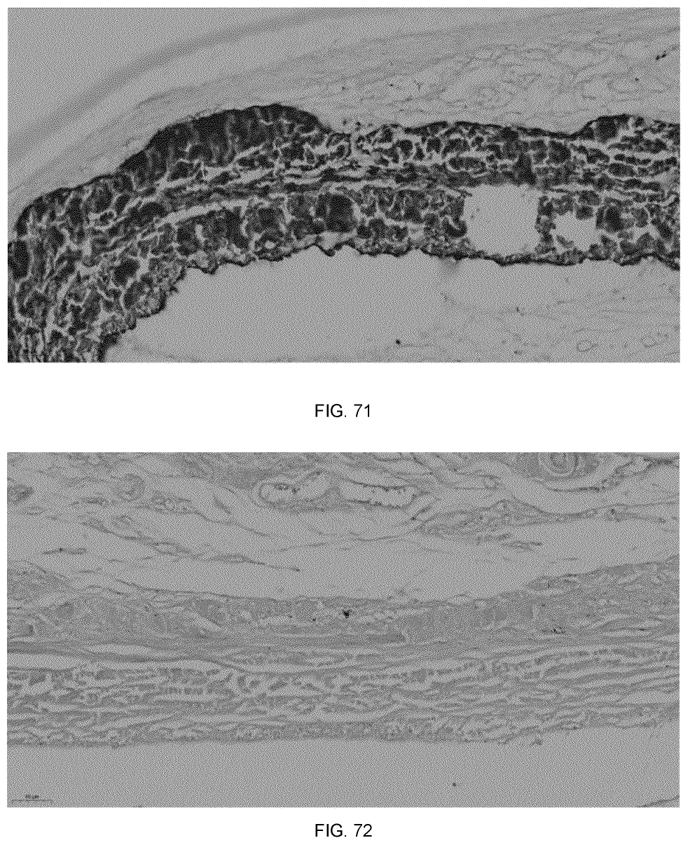

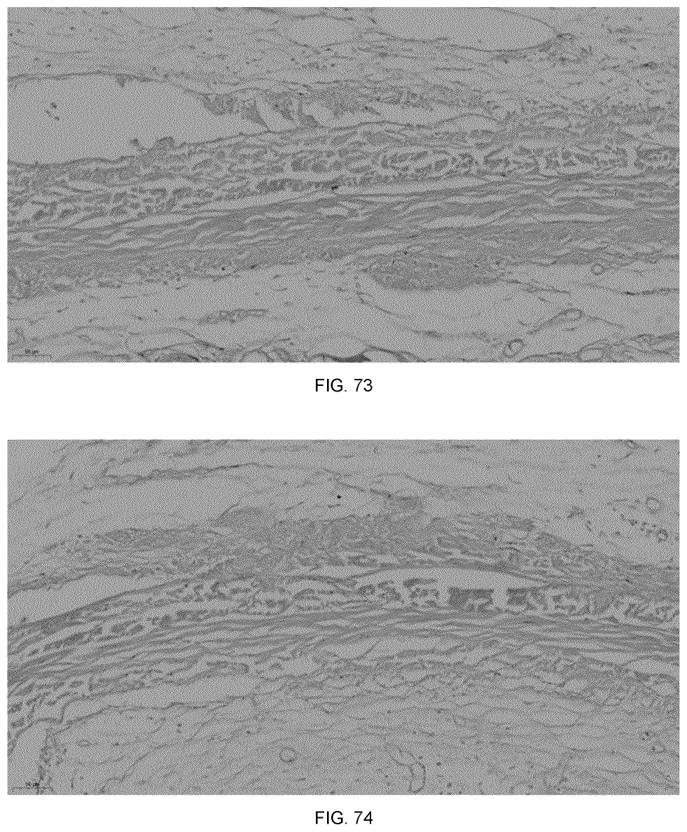

The alizarin red staining experiment was conducted on control group 9 (glutaraldehyde cross-linked porcine pericardium), sample 52, sample 53, and sample 56 after being implanted subcutaneously in rats for 30 days to directly observe the degree of calcification of samples. Images of the alizarin red staining results of sample slices after being implanted subcutaneously in rats for 30 days are shown in FIGS. 71 to 74, wherein the darker the color of the sample after alizarin red staining, the higher the degree of calcification. Compared with the alizarin red staining result of slice of control sample 9 (glutaraldehyde cross-linked porcine pericardium) (FIG. 71), the color of the alizarin red staining results of slices of sample 52 (FIG. 72), sample 53 (FIG. 73), sample 56 (FIG. 74) is obviously lighter, which directly indicates that the degree of calcification of sample 52, sample 53, and sample 56 is lower than that of the control group 9, that is, compared with the control group 9, sample 52, sample 53, and sample 56 have stronger anti-calcification effect. The alizarin red staining results of biological valve materials after being implanted subcutaneously in rats for 30 days show that the double-bond post-copolymerization and cross-linking method for preparing biological valve materials of the present disclosure can improve the anti-calcification performance of biological valves.

### Control Example 10

Meanwhile, the simple glutaraldehyde cross-linked group was set as the control group, wherein the porcine pericardium was soaked in 0.25% (w/w) glutaraldehyde for 72 hours at room temperature to prepare glutaraldehyde cross-linked porcine pericardium, which was recorded as control sample 10.

### Example 62

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 5% (v/v) glycidyl methacrylate at room temperature for double bonding modification of the glutaraldehyde cross-linked porcine pericardium for 48 hours, wherein the solvent of the double bonding solution was 20% (v/v) isopropanol aqueous solution.

After the double bonding modification, the double-bonded glutaraldehyde cross-linked porcine pericardium was washed with deionized water, and then soaked in 3% (v/v) 3-[N,N-dimethyl-[2-(2-methylprop-2-enoyloxy)ethyl]ammonium]propane-1-sulfonate solution for 2 hours.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the 3-[N,N-dimethyl-[2-(2-methylprop-2-enoyloxy)ethyl]ammonium]propane-1-sulfonate for 8 hours at 37° C., thereby obtaining double bond copolymerized and post-crosslinked porcine pericardium, which was recorded as sample 62.

### Example 63

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in a propanol aqueous solution of 6% (v/v) glycidyl acrylate at room temperature for double bonding modification of the glutaraldehyde cross-linked porcine pericardium for 72 hours, wherein the solvent of the double bonding solution was 20% (v/v) propanol aqueous solution.

After the double bonding modification, the double-bonded glutaraldehyde cross-linked porcine pericardium was washed with deionized water, and then soaked in 5% (w/v) 2-methacryloyloxyethyl phosphorylcholine solution for 1 hour;

An initiator was added to the above solution, which includes 20 mM potassium persulfate and 10 mM sodium sulfite, to further initiate polymerization between double bonds on the double-bonded glutaraldehyde cross-linked biological valve material and double bonds of the 2-methacryloyloxyethyl phosphorylcholine for 8 hours at 37° C., thereby obtaining double bond copolymerized and post-crosslinked porcine pericardium, which was recorded as sample 63.

### Example 64

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution containing both 2% (v/v) glycidyl acrylate and 4% (v/v) allyl glycidyl ether at room temperature for double bonding modification of the glutaraldehyde cross-linked porcine pericardium for 72 hours, wherein the solvent of the double bonding solution was 30% (v/v) ethanol aqueous solution.

After the double bonding modification, the double-bonded glutaraldehyde cross-linked porcine pericardium was washed with deionized water, and then soaked in a 5% (v/v) acrylamide solution for 3 hours.

An initiator was added to the above solution, which includes 20 mM potassium persulfate and 10 mM sodium sulfite, to further initiate polymerization between double bonds on the double-bonded glutaraldehyde cross-linked biological valve material and double bonds of the acrylamide for 8 hours at 37° C., thereby obtaining double bond copolymerized and post-crosslinked porcine pericardium, which was recorded as sample 64.

### Example 65

Freshly connected porcine pericardium was soaked in physiological saline, shaken and washed for 2 hours. Then, the porcine pericardium was soaked in 1% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 3% (v/v) glycidyl methacrylate and 2% (v/v) glycidyl acrylate at room temperature for double bonding modification of the glutaraldehyde cross-linked porcine pericardium for 48 hours, wherein the solvent of the double bonding solution was 25% (v/v) isopropanol aqueous solution.

After the double bonding modification, the double-bonded glutaraldehyde cross-linked porcine pericardium was washed with deionized water, and then soaked in a solution containing 1% (v/v) acrylamide and 1.5% (v/v) N-isopropylacrylamide for 1 hour;

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the acrylamide and N-isopropylacrylamide for 7 hours at 37° C., thereby obtaining double bond copolymerized and post-crosslinked porcine pericardium, which was recorded as sample 65.

### Example 66

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an ethanol aqueous solution of 4% (v/v) glycidyl methacrylate at room temperature for double bonding modification of the glutaraldehyde cross-linked porcine pericardium for 72 hours, wherein the solvent of the double bonding solution was 20% (v/v) ethanol aqueous solution.

After the double bonding modification, the double-bonded glutaraldehyde cross-linked porcine pericardium was washed with deionized water, and then soaked in 1.5% (v/v) N-isopropylacrylamide solution for 1 hour.

An initiator was added to the above solution, which includes 20 mM sodium persulfate and 7 mM sodium bisulfite, to further initiate polymerization between double bonds on the double-bonded glutaraldehyde cross-linked biological valve material and double bonds of the N-isopropylacrylamide for 7 hours at 37° C., thereby obtaining double bond copolymerized and post-crosslinked porcine pericardium, which was recorded as sample 66.

### Example 67

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isobutanol aqueous solution of 4% (v/v) glycidyl methacrylate at room temperature for double bonding modification of the glutaraldehyde cross-linked porcine pericardium for 72 hours, wherein the solvent of the double bonding solution was 15% (v/v) isobutanol aqueous solution.

After the double bonding modification, the double-bonded glutaraldehyde cross-linked porcine pericardium was washed with deionized water, and then soaked in 2.0% (w/v) sodium acrylate solution for 5 hours;

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the sodium acrylate for 12 hours at 37° C., thereby obtaining double bond copolymerized and post-crosslinked porcine pericardium, which was recorded as sample 67.

### Example 68

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 4% (v/v) glycidyl acrylate at room temperature for double bonding modification of the glutaraldehyde cross-linked porcine pericardium for 48 hours, wherein the solvent of the double bonding solution was 20% (v/v) methanol aqueous solution.

After the double bonding modification, the double-bonded glutaraldehyde cross-linked porcine pericardium was washed with deionized water, and then soaked in a solution containing 1.0% (v/v) hydroxyethyl methacrylate and 0.5% (w/v) 3-[N,N-dimethyl-[2-(2-methylprop-2-enoyloxy)ethyl]ammonium]propane-1-sulfonate for 1 hour.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the hydroxyethyl methacrylate and 3-[N,N-dimethyl-[2-(2-methylprop-2-enoyloxy)ethyl]ammonium]propane-1-sulfonate for 8 hours at 37° C., thereby obtaining double bond copolymerized and post-crosslinked porcine pericardium, which was recorded as sample 68.

### Example 69

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an ethylene glycol aqueous solution of 5% (v/v) glycidyl methacrylate at room temperature for double bonding modification of the glutaraldehyde cross-linked porcine pericardium for 72 hours, wherein the solvent of the double bonding solution was 25% (v/v) ethylene glycol aqueous solution.

After the double bonding modification, the double-bonded glutaraldehyde cross-linked porcine pericardium was washed with deionized water, and then soaked in 5% (v/v) N-(hydroxymethyl)acrylamide solution for 5 hours.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the N-(hydroxymethyl)acrylamide for 12 hours at 37° C., thereby obtaining double bond copolymerized and post-crosslinked porcine pericardium, which was recorded as sample 69.

### Example 70

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in a propanol aqueous solution of 7% (v/v) glycidyl acrylate at room temperature for double bonding modification of the glutaraldehyde cross-linked porcine pericardium for 60 hours, wherein the solvent of the double bonding solution was 30% (v/v) propanol aqueous solution.

After the double bonding modification, the double-bonded glutaraldehyde cross-linked porcine pericardium was washed with deionized water, and then soaked in 1.0% (v/v) N-(methoxymethyl)methacrylamide solution for 5 hours;

An initiator was added to the above solution, which includes 20 mM ammonium persulfate and 1 mM tetramethylethylenediamine, to further initiate polymerization between double bonds on the double-bonded glutaraldehyde cross-linked biological valve material and double bonds of the N-(methoxymethyl)methacrylamide for 12 hours at 37° C., thereby obtaining double bond copolymerized and post-crosslinked porcine pericardium, which was recorded as sample 70.

### Example 71

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution containing 4% (v/v) glycidyl methacrylate and 2% (v/v) glycidyl acrylate at room temperature for double bonding modification of the glutaraldehyde cross-linked porcine pericardium for 84 hours, wherein the solvent of the double bonding solution was 25% (v/v) ethanol aqueous solution.

After the double bonding modification, the double-bonded glutaraldehyde cross-linked porcine pericardium was washed with deionized water, and then soaked in 1% (v/v) acrylamide and 1.50% (w/v) 2-methacryloyloxyethyl phosphorylcholine solution for 3 hours.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the acrylamide and 2-methacryloyloxyethyl phosphorylcholine for 7 hours at 37° C., thereby obtaining double bond copolymerized and post-crosslinked porcine pericardium, which was recorded as sample 71.

### Performance Characterization of the Samples of Examples 62 to 71 and Control Example 10:

In order to characterize the change in the cross-linking degree of glutaraldehyde cross-linked biological valve materials before and after double-bond post-copolymerization and cross-linking treatment, the thermal stability and cross-linking degree of biological valve materials were characterized by measuring the thermal shrinkage temperature of biological valve materials; the stability of biological valve materials were characterized through enzyme degradation experiment; the degree of calcification (anti-calcification performance) of the samples were characterized through rat subcutaneous implantation experiment; the elasticity of biological valve materials were characterized by measuring their elastic angles; the hydrophilicity of biological valve materials were characterized through water contact angle test; and the anti-thrombotic properties of the materials were characterized through blood adhesion experiment.

### Thermal Shrinkage Temperature Measurement

The biological valve material was cut into a circular sheet with a diameter of 0.6 cm, which was placed in a crucible after drying, and then the thermal shrinkage temperature of the biological valve material was measured on a differential scanning calorimeter in the range of 40-120° C. at a heating rate of 10° C./min. Thermal stability and cross-linking degree of the biological valve material were characterized by measuring the thermal shrinkage temperature. The higher the thermal shrinkage temperature, the higher the thermal stability and cross-linking degree.

TABLE 17
Thermal shrinkage temperature of biological valve materials

| Sample name | Thermal shrinkage temperature (° C.) |
|---|---|
| Control group 10 | 86.7 |
| (glutaraldehyde cross- | |
| linked porcine pericardium) | |
| Sample 62 | 89.4 |
| Sample 64 | 91.8 |
| Sample 67 | 90.5 |
| Sample 69 | 92.4 |

Through the thermal shrinkage temperature measurement of sample 62, sample 64, sample 67, sample 69 and control group 10 (glutaraldehyde cross-linked porcine pericardium), it can be seen that, as shown in Table 17, the thermal shrinkage temperatures of sample 62, sample 64, sample 67 and sample 69 are all higher than that of the control group 10 (glutaraldehyde cross-linked porcine pericardium), that is, the thermal stability and cross-linking degree of sample 62, sample 64, sample 67 and sample 69 are all higher than those in the control group (glutaraldehyde cross-linked porcine pericardium). The experimental results of the thermal shrinkage temperature measurement show that the double-bond post-functionalized copolymerization and cross-linking method for preparing biological valve materials of the present disclosure can improve the thermal stability and cross-linking degree of biological valves.

### Water Contact Angle Test

The biological valve materials were cut into 1*1 cm² sheets, frozen overnight at −80° C., then transferred to a freeze-dryer and freeze-dried for 48 hours. Then the sheets were taken out and placed on a water contact angle tester to measure the water contact angles of the materials to characterize the hydrophilicity of the materials. The smaller the water contact angle, the more hydrophilic the biological valve material is.

TABLE 18
Water contact angle of biological valve materials

| Sample name | Water contact angle (°) |
|---|---|
| Control group 10 (glutaraldehyde cross- | 67 |
| linked porcine pericardium) | |
| Sample 62 | 26 |
| Sample 63 | 42 |
| Sample 68 | 45 |
| Sample 71 | 33 |

The water contact angle test results are shown in Table 18. Compared with the control group 10 (glutaraldehyde cross-linked porcine pericardium), the water contact angles of sample 62, sample 63, sample 68, and sample 71 are all significantly decreased, that is, sample 62, sample 63, sample 68, and sample 71 are more hydrophilic than control group 10 (glutaraldehyde cross-linked porcine pericardium), which indicates that the double-bond post-functionalized copolymerization and cross-linking method for preparing biological valve materials can improve the hydrophilicity of biological valves.

### Blood Adhesion Experiment

The biological valve material was cut into a circular sheet with a diameter of 1 cm and transferred to a 48-well plate. Then 0.5 mL of fresh rabbit blood was added to the surface of the material to fully contact the blood for blood adhesion experiments. After 1.5 hours of contact with blood, the biological valve material was removed from the blood and washed three times with physiological saline. The cleaned biological valve material was soaked in 2.5% (w/v) glutaraldehyde solution for 2 hours. After fixation, the biological valve material was dehydrated with ethanol at gradient concentrations (50%, 75%, 90% and 100%, v/v), then sprayed with gold, and finally placed on a scanning electron microscope to observe and photograph the blood adhesion to characterize anti-thrombotic properties.

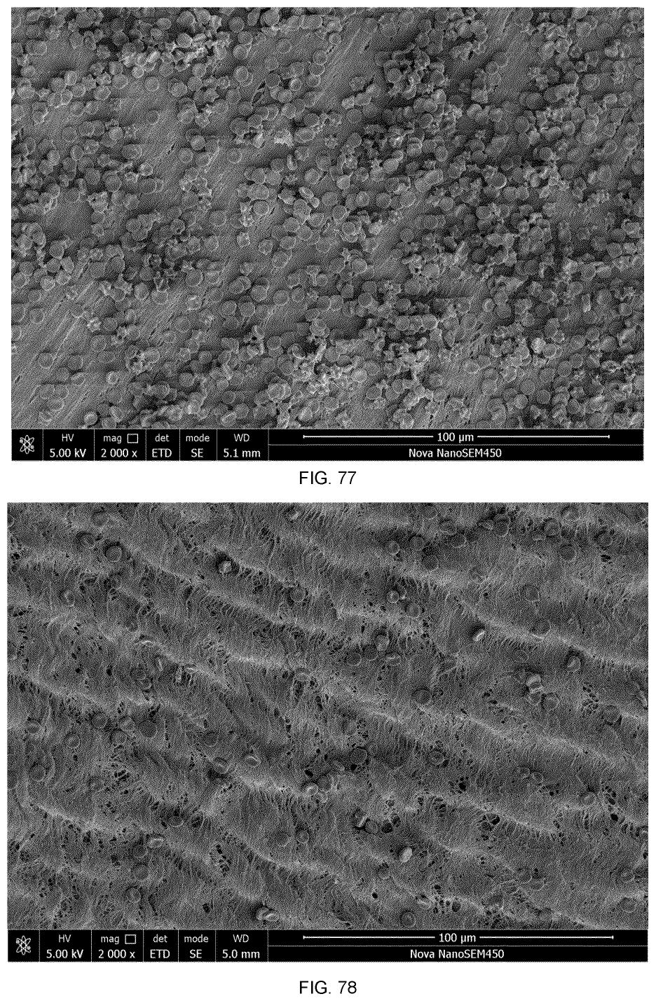

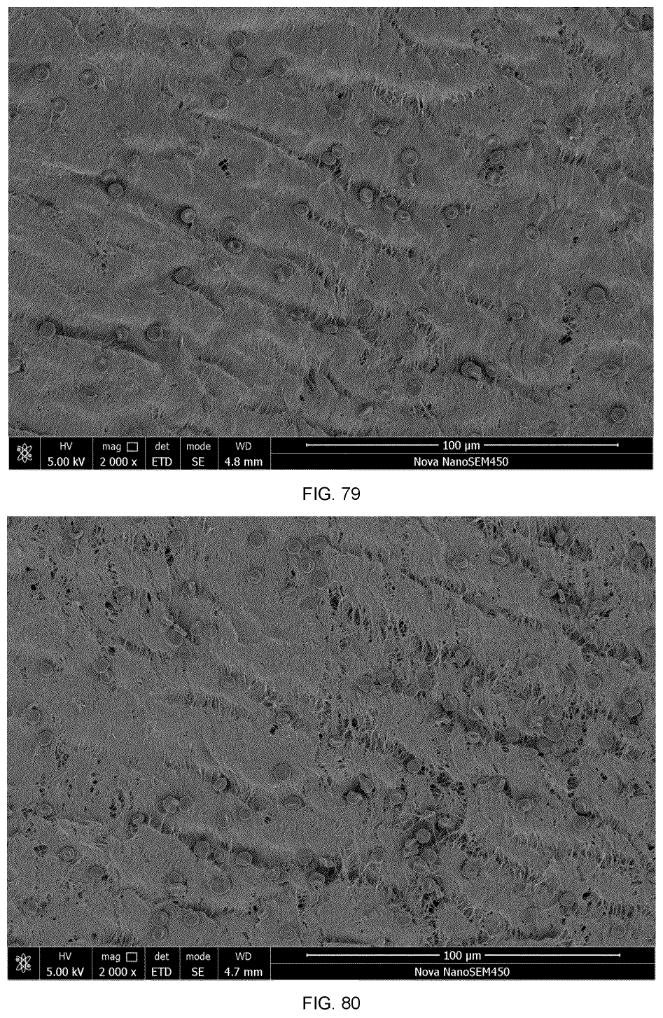

Result analysis: as shown in FIGS. 77 to 80, after contact with blood, a large amount of red blood cells and platelet adhesion was observed in control group 10 (glutaraldehyde cross-linked porcine pericardium) (FIG. 77), while only a small amount of red blood cell adhesion was observed in sample 62 (FIG. 78), sample 63 (FIG. 79), and sample 68 (FIG. 80); the blood cell adhesion on Sample 62, Sample 63, and Sample 68 was low, reducing the interaction between blood and biological valve materials, which further reduces the risk of thrombosis on the biological valve material, that is, sample 62, sample 63, and sample 68 have better anti-thrombotic properties than control group 10 (glutaraldehyde cross-linked porcine pericardium); blood adhesion experiments show that, the double-bond post-functionalized copolymerization and cross-linking method for preparing biological valve materials can improve the anti-thrombotic properties of biological valves.

### Elasticity Test Experiment

The biological valve material with uniform thickness was cut into a rectangular sample of 1*4.6 cm², which was clamped horizontally along the midline of the long side thereof. The angle at which the sample sag relative to the horizontal plane of the midline was tested to characterize the elasticity of the sample. The smaller the angle, the higher the elasticity.

TABLE 19
Elastic angle of biological valve materials

| Sample name | Elastic angle (°) |
|---|---|
| Control group 10 (glutaraldehyde cross- | 60 |
| linked porcine pericardium) | |
| Sample 62 | 50 |
| Sample 64 | 35 |
| Sample 67 | 45 |
| Sample 69 | 30 |

The elasticity test experiment was conducted on sample 62, sample 64, sample 67, sample 69 and control group 3 (glutaraldehyde cross-linked porcine pericardium) to characterize their elasticity. The elasticity test results are shown in Table 19. Compared with the control group 10 (glutaraldehyde cross-linked porcine pericardium), the elastic angles of sample 62, sample 64, sample 67, and sample 69 are lower, indicating that their elasticity is significantly improved than the control group (glutaraldehyde cross-linked porcine pericardium). The double-bond post-functionalized copolymerization and cross-linking method for preparing biological valve materials can improve the elasticity of biological valve materials, which is conducive to rapid recovery of shape after transcatheter implantation.

### Enzyme Degradation Experiment

The collagenase degradation weight loss rates of sample 62, sample 64, sample 67, sample 69 and control group 10 were measured using the same method as the aforementioned enzyme degradation experiment.

TABLE 20
Enzymatic degradation weight loss rate of biological valve materials

| | Enzymatic degradation |
|---|---|
| Tested sample | weight loss rate (%) |
| Control group (glutaraldehyde cross-linked | 6.35 ± 0.89 |
| porcine pericardium) | |
| Example 62 | 3.12 ± 0.30 |
| Example 64 | 2.65 ± 0.47 |
| Example 67 | 4.90 ± 0.45 |
| Example 69 | 1.15 ± 0.26 |

As shown in Table 20, the enzyme degradation experiment was conducted on sample 62, sample 64, sample 67, sample 69 and control group 10 (glutaraldehyde cross-linked porcine pericardium) to characterize the cross-linking efficiency of samples, and the enzymatic degradation weight loss rates of samples after sample 62, sample 64, sample 67, sample 69 and control group 10 (glutaraldehyde cross-linked porcine pericardium) were treated with collagenase I were calculated as shown in Table 20. The enzymatic degradation weight loss rates of sample 62, sample 64, sample 67, and sample 69 were all lower than that of the control group 10 (glutaraldehyde cross-linked porcine pericardium), which indicates that sample 62, sample 64, sample 67, and sample 69 have higher stability than the control group (glutaraldehyde cross-linked porcine pericardium). The results of the enzyme degradation experiment show that the double-bond post-functionalized copolymerization and cross-linking method for preparing biological valve materials of the present disclosure can improve the stability of biological valves.

### Anti-Calcification Test

The biological valve materials of sample 62, sample 63, sample 69 and control group 10 (glutaraldehyde cross-linked porcine pericardium) were cut into 1*1 cm² sheets, and the anti-calcification test was conducted using the same method as the aforementioned anti-calcification test.

TABLE 21
Calcium content of biological valve materials after
subcutaneous implantation in rats for 30 days

| Sample name | Calcium content (mg/g) |
|---|---|
| Control group 10 (glutaraldehyde cross- | 73.8 ± 11.3 |
| linked porcine pericardium) | |
| Sample 62 | 21.3 ± 2.7 |
| Sample 63 | 14.1 ± 5.4 |
| Sample 69 | 5.9 ± 0.87 |

The calcium content of sample 62, sample 63, sample 69 and control group 10 (glutaraldehyde cross-linked porcine pericardium) after being implanted subcutaneously in rats for 30 days was measured to characterize the degree of calcification of samples. As shown in Table 21, the calcium contents of sample 62, sample 63, and sample 69 after being implanted subcutaneously in rats for 30 days were all lower than that of the control group (glutaraldehyde cross-linked porcine pericardium). This result indicates that the double-bond post-functionalized copolymerization and cross-linking method for preparing biological valve materials of the present disclosure can improve the anti-calcification properties of biological valves.

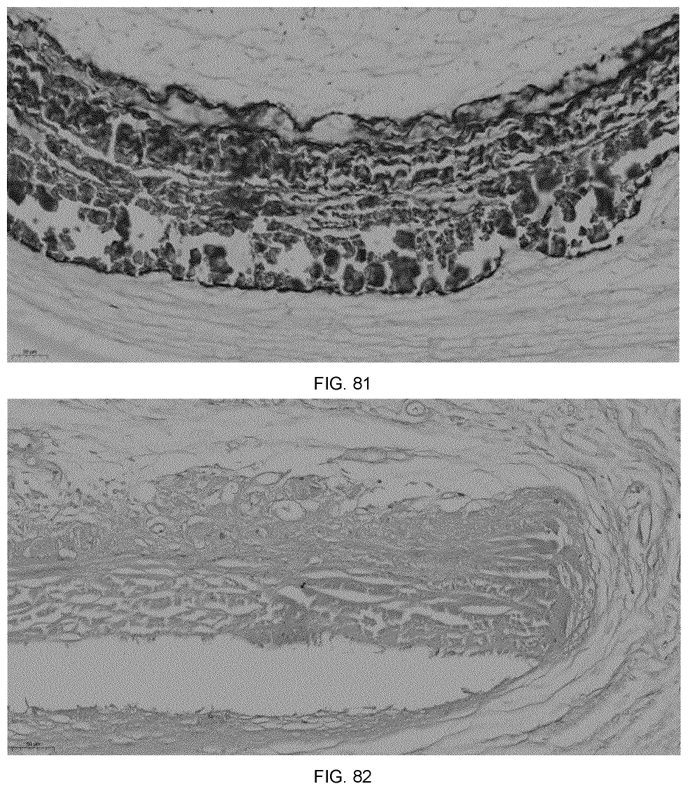

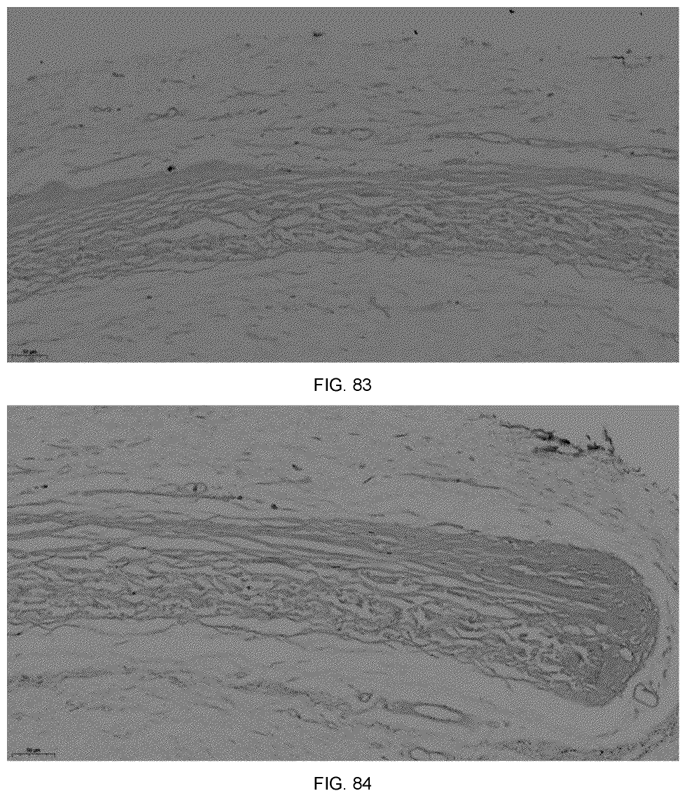

The alizarin red staining experiment was conducted on control group 10 (glutaraldehyde cross-linked porcine pericardium), sample 62, sample 63, and sample 69 after being implanted subcutaneously in rats for 30 days to directly observe the degree of calcification of samples. Images of the alizarin red staining results of sample slices after being implanted subcutaneously in rats for 30 days are shown in FIGS. 81 to 8, wherein the darker the color of the sample after alizarin red staining, the higher the degree of calcification. Compared with the alizarin red staining result of slice of control sample 10 (glutaraldehyde cross-linked porcine pericardium) (FIG. 81), the color of the alizarin red staining results of slices of Example 62 (FIG. 82), Example 63 (FIG. 83), and Example 69 (FIG. 84) is obviously lighter, which directly indicates that the degree of calcification of sample 62, sample 63, and sample 69 is lower than that of the control group 10, that is, sample 62, sample 63, and sample 69 have stronger anti-calcification effect. The alizarin red staining results of biological valve materials after being implanted subcutaneously in rats for 30 days show that the double-bond post-functionalized copolymerization and cross-linking method for preparing biological valve materials of the present disclosure can improve the anti-calcification performance of biological valves.

### Example 72

Freshly collected porcine pericardium was washed with distilled water for 2 hours at 4° C. with shaking at 100 RPM, and then soaked in 0.30% (w/w) glutaraldehyde solution at room temperature for 48 hours, wherein the biological valve was cross-linked with glutaraldehyde to obtain glutaraldehyde cross-linked porcine pericardium.

The porcine pericardium was further washed with deionized water, and then soaked in an ethanol aqueous solution of 4% (v/v) glycidyl methacrylate at room temperature for double bonding modification of the glutaraldehyde cross-linked porcine pericardium for 72 hours, wherein the solvent of the double bonding solution was 20% (v/v) ethanol aqueous solution.

After the double bonding modification, an initiator of ammonium persulfate and sodium bisulfite was added to initiate polymerization of double bonds on the double-bonded glutaraldehyde cross-linked porcine pericardium for 8 hours at 37° C., thereby obtaining double-bond post-crosslinked porcine pericardium, wherein the concentration of ammonium persulfate was 20 mM and the concentration of sodium bisulfite was 5 mM.

The double bond copolymerized and post-crosslinked porcine pericardium material was soaked in 70% ethanol aqueous solution for 20 minutes, and then soaked in a drying solution (80% glycerol, 2% water, 18% ethanol) at room temperature for 1.5 hours. The excess glycerol was removed from the surface of the porcine pericardium material, which was sterilized with ethylene oxide, and recorded as sample 72.

### Example 73

Freshly connected porcine pericardium was soaked in physiological saline, shaken and washed for 2 hours. Then, the porcine pericardium was soaked in 0.625% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 5% (v/v) glycidyl methacrylate at room temperature for double bonding modification of the glutaraldehyde cross-linked porcine pericardium for 72 hours, wherein the solvent of the double bonding solution was 18% (v/v) isopropanol aqueous solution.

After the double bonding modification, the double-bonded glutaraldehyde cross-linked porcine pericardium was washed with deionized water, and then soaked in 2% (v/v) polyethylene glycol diacrylate aqueous solution for 2 hours.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the polyethylene glycol diacrylate for 8 hours at 37° C., thereby obtaining double bond copolymerized and post-crosslinked porcine pericardium.

The double bond copolymerized and post-crosslinked porcine pericardium material was soaked in 60% isopropanol aqueous solution for 45 minutes, and then soaked in a solution consisting of 10% glycerol, 3% polyethylene glycol (Mn=200), and 87% ethanol at room temperature for 3 hours. The excess glycerol was removed from the surface of the porcine pericardium material, which was sterilized with ethylene oxide, and recorded as sample 73.

### Example 74

Freshly connected porcine pericardium was soaked in physiological saline, shaken and washed for 2 hours. Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an ethylene glycol aqueous solution of 5% (v/v) glycidyl methacrylate at room temperature for double bonding modification of the glutaraldehyde cross-linked porcine pericardium for 72 hours, wherein the solvent of the double bonding solution was 25% (v/v) ethylene glycol aqueous solution.

After the double bonding modification, the double-bonded glutaraldehyde cross-linked porcine pericardium was washed with deionized water, and then soaked in 5% (v/v) N-(hydroxymethyl)acrylamide solution for 5 hours.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the N-(hydroxymethyl)acrylamide for 12 hours at 37° C., thereby obtaining double bond copolymerized and post-crosslinked porcine pericardium. The double bond copolymerized and post-crosslinked porcine pericardium material was soaked in 60% isopropanol aqueous solution for 45 minutes, and then soaked in a solution consisting of 10% glycerol, 3% polyethylene glycol (Mn=200), and 87% ethanol at room temperature for 3 hours. The excess glycerol was removed from the surface of the porcine pericardium material, which was sterilized with ethylene oxide, and recorded as sample 74.

### Example 75

Fresh porcine pericardium was placed in PBS solution of sodium deoxycholate (surfactant) with a mass fraction of 0.5%, shaken at room temperature for 4 hours, and then washed with sodium chloride aqueous solution (i.e. physiological saline) with a mass fraction of 0.9% three times.

Then, the porcine pericardium was soaked in 0.25% (w/w) glutaraldehyde solution at room temperature, and shaken for 72 hours to treat the porcine pericardium by cross-linking with glutaraldehyde to prepare glutaraldehyde cross-linked porcine pericardium.

The porcine pericardium was further washed with deionized water, and then soaked in an isopropanol aqueous solution of 5% (v/v) glycidyl methacrylate at room temperature for double bonding modification of the glutaraldehyde cross-linked porcine pericardium for 48 hours, wherein the solvent of the double bonding solution was 20% (v/v) isopropanol aqueous solution.

After the double bonding modification, the double-bonded glutaraldehyde cross-linked porcine pericardium was washed with deionized water, and then soaked in 3% (v/v) 3-[N,N-dimethyl-[2-(2-methylprop-2-enoyloxy)ethyl]ammonium]propane-1-sulfonate solution for 2 hours.

An initiator was added to the above solution, which includes 20 mM ammonium persulfate and 10 mM sodium bisulfite, to further initiate polymerization between double bonds on the double-bonded glutaraldehyde cross-linked biological valve material and double bonds of the 3-[N,N-dimethyl-[2-(2-methylprop-2-enoyloxy)ethyl]ammonium]propane-1-sulfonate for 8 hours at 37° C., thereby obtaining double bond copolymerized and post-crosslinked porcine pericardium, which was recorded as sample 75.

### Example 76

Freshly collected porcine pericardium was washed with distilled water for 2 hours at 4° C. with shaking at 100 RPM until there is no visible adherent non-pericardium or non-collagenous tissue. Then the cleaned biological material was soaked in a 10 mM DL-2-amino-4-pentenoic acid aqueous solution at 37° C. for 12 hours to ensure sufficient physical permeation of the DL-2-amino-4-pentenoic acid aqueous solution. Then, glutaraldehyde was added to a concentration of 10 mM, wherein the porcine pericardium was soaked for 24 hours at 37° C. with shaking at 100 RPM. Then, the porcine pericardium was soaked and washed with distilled water to remove unreacted DL-2-amino-4-pentenoic acid and glutaraldehyde. The cleaned biological materials was soaked in 1% polyethylene glycol diacrylate aqueous solution at 37° C. for 12 hours to ensure sufficient physical permeation of polyethylene glycol diacrylate. An initiator of ammonium persulfate and sodium bisulfite was added to initiate the reaction for 24 hours at 37° C., wherein the molar concentrations of ammonium persulfate and sodium bisulfite are both 10 mM, thereby obtaining a biological valve material, which was recorded as sample 76.

### Example 77

Freshly collected porcine pericardium was washed with distilled water for 2 hours at 4° C. with shaking at 100 RPM until there is no visible adherent non-pericardium or non-collagenous tissue. Then the cleaned biological material was soaked in a 100 mM DL-2-amino-4-pentenoic acid aqueous solution at 37° C. for 12 hours to ensure sufficient physical permeation of the DL-2-amino-4-pentenoic acid aqueous solution. Then, glutaraldehyde was added to a concentration of 500 mM, wherein the porcine pericardium was soaked for 24 hours at 37° C. with shaking at 100 RPM. Then, the porcine pericardium was soaked and washed with distilled water to remove unreacted DL-2-amino-4-pentenoic acid and glutaraldehyde. The cleaned biological materials was soaked in 10% polyethylene glycol diacrylate aqueous solution at 37° C. for 12 hours to ensure sufficient physical permeation of polyethylene glycol diacrylate. An initiator of ammonium persulfate and sodium bisulfite was added to initiate the reaction for 24 hours at 37° C., wherein the molar concentrations of ammonium persulfate and sodium bisulfite are both 100 mM, thereby obtaining a biological valve material, which was recorded as sample 77.

### Example 78

Freshly collected porcine pericardium was washed with distilled water for 2 hours at 4° C. with shaking at 100 RPM until there is no visible adherent non-pericardium or non-collagenous tissue. Then the cleaned biological material was soaked in a 50 mM DL-2-amino-4-pentenoic acid aqueous solution at 37° C. for 12 hours to ensure sufficient physical permeation of the DL-2-amino-4-pentenoic acid aqueous solution. Then, glutaraldehyde was added to a concentration of 220 mM, wherein the porcine pericardium was soaked for 24 hours at 37° C. with shaking at 100 RPM. Then, the porcine pericardium was soaked and washed with distilled water to remove unreacted DL-2-amino-4-pentenoic acid and glutaraldehyde. The cleaned biological materials was soaked in 6% polyethylene glycol diacrylate aqueous solution at 37° C. for 12 hours to ensure sufficient physical permeation of polyethylene glycol diacrylate. An initiator of ammonium persulfate and sodium bisulfite was added to initiate the reaction for 24 hours at 37° C., wherein the molar concentrations of ammonium persulfate and sodium bisulfite are both 60 mM, thereby obtaining a biological valve material, which was recorded as sample 78.

The biological valve materials from the above Examples 76 to 78 and Control example 1 were subjected to lactate dehydrogenase relative activity measurement. The test method was the same as that in Example 1, and the test results are shown in Table 22.

TABLE 22

| | Lactate dehydrogenase relative activity |
|---|---|
| Example 76 | 0.236 ± 0.031 |
| Example 77 | 0.131 ± 0.033 |
| Example 78 | 0.123 ± 0.030 |
| Control example 1 | 0.410 ± 0.072 |

### Example 79

Modified Hyaluronic Acid and Modified Polylysine were Prepared Respectively:

2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

Freshly collected porcine pericardium was washed with distilled water for 2 hours at 4° C. with shaking at 100 RPM until there is no visible adherent non-pericardium or non-collagenous tissue. Then, the porcine pericardium was soaked in 180 mM modified polylysine aqueous solution at room temperature for 24 hours to ensure near-saturated physical permeation, thereby introducing as much partially double-bonded polylysine as possible. Then, glutaraldehyde solution was added to a mass concentration of 2.5% for reaction for 24 hours at 37° C. on a shaker. The pericardium material was then taken out and cleaned and soaked in a 10 mg/ml modified hyaluronic acid aqueous solution at room temperature for 12 hours to ensure near-saturated physical permeation, thereby introducing as much double-bonded hyaluronic acid as possible. The pericardium material was then soaked in an initiator of 2.5% ammonium persulfate and 0.25% N,N,N′,N′-tetramethylethylenediamine at 37° C. for 12 hours, and finally soaked and washed with distilled water to remove ungrafted double-bonded hyaluronic acid, thereby obtaining a biological valve material, which was recorded as sample 79.

### Example 80

Modified Hyaluronic Acid and Modified Polylysine were Prepared Respectively:

2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

Freshly collected porcine pericardium was washed with distilled water for 2 hours at 4° C. with shaking at 100 RPM until there is no visible adherent non-pericardium or non-collagenous tissue. Then, the porcine pericardium was soaked in 180 mM modified polylysine aqueous solution at room temperature for 24 hours to ensure near-saturated physical permeation, thereby introducing as much partially double-bonded polylysine as possible. Then, glutaraldehyde solution was added to a mass concentration of 2.5% for reaction for 24 hours at 37° C. on a shaker. The pericardium material was then taken out and cleaned and soaked in a 30 mg/ml modified hyaluronic acid aqueous solution at room temperature for 12 hours to ensure near-saturated physical permeation, thereby introducing as much double-bonded hyaluronic acid as possible. The pericardium material was then soaked in an initiator of 2.5% ammonium persulfate and 0.25% N,N,N′,N′-tetramethylethylenediamine at 37° C. for 12 hours, and finally soaked and washed with distilled water to remove ungrafted double-bonded hyaluronic acid, thereby obtaining a biological valve material, which was recorded as sample 80.

### Example 81

Modified Hyaluronic Acid and Modified Polylysine were Prepared Respectively:

2 g of sodium hyaluronate with a molecular weight of 10,000 was weighed, and dissolved in 20 ml of PBS, and then 6.5 ml of glycidyl methacrylate and 4.5 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Polylysine was dissolved in deionized water, and then glycidyl methacrylate was added at a molar ratio of (1:1.5) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

Freshly collected porcine pericardium was washed with distilled water for 2 hours at 4° C. with shaking at 100 RPM until there is no visible adherent non-pericardium or non-collagenous tissue. Then, the porcine pericardium was soaked in 180 mM modified polylysine aqueous solution at room temperature for 24 hours to ensure near-saturated physical permeation, thereby introducing as much partially double-bonded polylysine as possible. Then, glutaraldehyde solution was added to a mass concentration of 2.5% for reaction for 24 hours at 37° C. on a shaker. The pericardium material was then taken out and cleaned and soaked in a 100 mg/ml modified hyaluronic acid aqueous solution at room temperature for 12 hours to ensure near-saturated physical permeation, thereby introducing as much double-bonded hyaluronic acid as possible. The pericardium material was then soaked in an initiator of 2.5% ammonium persulfate and 0.25% N,N,N′,N′-tetramethylethylenediamine at 37° C. for 12 hours, and finally soaked and washed with distilled water to remove ungrafted double-bonded hyaluronic acid, thereby obtaining a biological valve material, which was recorded as sample 81.

### Example 82

Modified Hyaluronic Acid and Modified Polylysine were Prepared Respectively:

2 g of sodium hyaluronate with a molecular weight of 10,000 was weighed, and dissolved in 20 ml of PBS, and then 12 ml of glycidyl methacrylate and 6 ml of triethylamine were added in sequence; the mixture was placed on a shaker at 37° C. for 5 days; finally, a dialysis bag with a molecular weight cutoff of 5000 was used for dialysis for 5 days, and then freeze-dried to obtain double-bonded hyaluronic acid.

Polylysine was dissolved in deionized water, and then glycidyl methacrylate was added at a molar ratio of (1:1) (glycidyl methacrylate:amino group); the mixture was placed on a shaker at 37° C. for 5 days; finally, a dialysis bag with a molecular weight cutoff of 1000 was used for dialysis for 5 days, and then freeze-dried to obtain partially double-bonded polylysine.

Freshly collected porcine pericardium was washed with distilled water at 4° C. with shaking at 100 RPM until there is no visible adherent non-pericardium or non-collagenous tissue. Then, the porcine pericardium was soaked in 500 mM modified polylysine aqueous solution at room temperature for 12 hours to ensure near-saturated physical permeation, thereby introducing as much partially double-bonded polylysine as possible. Then, glutaraldehyde solution was added to a mass concentration of 2.5% for reaction for 24 hours at 37° C. on a shaker. The pericardium material was then taken out and cleaned and soaked in a 20 mg/ml modified hyaluronic acid aqueous solution at room temperature for 24 hours to ensure near-saturated physical permeation, thereby introducing as much double-bonded hyaluronic acid as possible. The pericardium material was then soaked in an initiator of 2% ammonium persulfate and 0.2% N,N,N′,N′-tetramethylethylenediamine at 37° C. for 24 hours, and finally soaked and washed with distilled water to remove ungrafted double-bonded hyaluronic acid, thereby obtaining a biological valve material, which was recorded as sample 82.

### Example 83

Modified Hyaluronic Acid and Modified Polylysine were Prepared Respectively:

2 g of sodium hyaluronate with a molecular weight of 10,000 was weighed, and dissolved in 20 ml of PBS, and then 6 ml of glycidyl methacrylate and 4 ml of triethylamine were added in sequence; the mixture was placed on a shaker at 37° C. for 7 days; finally, a dialysis bag with a molecular weight cutoff of 5000 was used for dialysis for 7 days, and then freeze-dried to obtain double-bonded hyaluronic acid.

Polylysine was dissolved in deionized water, and then glycidyl methacrylate was added at a molar ratio of (1.5:1) (glycidyl methacrylate:amino group); the mixture was placed on a shaker at 37° C. for 7 days; finally, a dialysis bag with a molecular weight cutoff of 1000 was used for dialysis for 7 days, and then freeze-dried to obtain partially double-bonded polylysine.

Freshly collected porcine pericardium was washed with distilled water at 4° C. with shaking at 100 RPM until there is no visible adherent non-pericardium or non-collagenous tissue. Then, the porcine pericardium was soaked in 100 mM modified polylysine aqueous solution at room temperature for 48 hours to ensure near-saturated physical permeation, thereby introducing as much partially double-bonded polylysine as possible. Then, glutaraldehyde solution was added to a mass concentration of 2.5% for reaction for 24 hours at 37° C. on a shaker. The pericardium material was then taken out and cleaned and soaked in a 60 mg/ml modified hyaluronic acid aqueous solution at room temperature for 24 hours to ensure near-saturated physical permeation, thereby introducing as much double-bonded hyaluronic acid as possible. The pericardium material was then soaked in an initiator of 5% ammonium persulfate and 0.3% N,N,N′,N′-tetramethylethylenediamine at 37° C. for 24 hours, and finally soaked and washed with distilled water to remove ungrafted double-bonded hyaluronic acid, thereby obtaining a biological valve material, which was recorded as sample 83.

### Example 84

Modified Hyaluronic Acid and Modified Polylysine were Prepared Respectively:

2 g of sodium hyaluronate with a molecular weight of 10,000 was weighed, and dissolved in 20 ml of PBS, and then 10 ml of glycidyl methacrylate and 8 ml of triethylamine were added in sequence; the mixture was placed on a shaker at 37° C. for 6 days; finally, a dialysis bag with a molecular weight cutoff of 5000 was used for dialysis for 6 days, and then freeze-dried to obtain double-bonded hyaluronic acid.

Polylysine was dissolved in deionized water, and then glycidyl methacrylate was added at a molar ratio of (1:1.5) (glycidyl methacrylate:amino group); the mixture was placed on a shaker at 37° C. for 6 days; finally, a dialysis bag with a molecular weight cutoff of 1000 was used for dialysis for 6 days, and then freeze-dried to obtain partially double-bonded polylysine.

Freshly collected porcine pericardium was washed with distilled water at 4° C. with shaking at 100 RPM until there is no visible adherent non-pericardium or non-collagenous tissue. Then, the porcine pericardium was soaked in 200 mM modified polylysine aqueous solution at room temperature for 24 hours to ensure near-saturated physical permeation, thereby introducing as much partially double-bonded polylysine as possible. Then, glutaraldehyde solution was added to a mass concentration of 2.5% for reaction for 24 hours at 37° C. on a shaker. The pericardium material was then taken out and cleaned and soaked in a 35 mg/ml modified hyaluronic acid aqueous solution at room temperature for 24 hours to ensure near-saturated physical permeation, thereby introducing as much double-bonded hyaluronic acid as possible. The pericardium material was then soaked in an initiator of 3% ammonium persulfate and 0.4% N,N,N′,N′-tetramethylethylenediamine at 37° C. for 12 hours, and finally soaked and washed with distilled water to remove ungrafted double-bonded hyaluronic acid, thereby obtaining a biological valve material, which was recorded as sample 84.

The biological valve materials from the above Examples 79 to 81 and Control example 2 were subjected to lactate dehydrogenase relative activity measurement. The test method was the same as that in Example 2, and the test results are shown in Table 23.

TABLE 23

| | Lactate dehydrogenase activity |
|---|---|
| Example 79 | 0.29 |
| Example 80 | 0.26 |
| Example 81 | 0.24 |
| Control example 2 | 0.41 |

The above-described embodiments only illustrate several embodiments of the present disclosure, and the description thereof is specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure. Therefore, the protection scope of the present disclosure should be determined by the appended claims.

1. A preparation method for a biological valve material, comprising:
step S100: treating a biological material with a first treatment solution and a second treatment solution in sequence to obtain a pretreated biological material chemically grafted with first carbon-carbon double bonds; wherein the first treatment solution contains a reagent A, and the second treatment solution contains a reagent B; the reagent A is a first functional monomer with a first carbon-carbon double bond, the first functional monomer further comprises an active group for participating in a chemical reaction, and the active group is an active group for reacting with an aldehyde group; and the reagent B is an aldehyde-based cross-linking agent; and
step S200: performing polymerization of carbon-carbon double bonds under an action of an initiator to obtain the biological valve material.

2. The preparation method of claim 1, wherein the first functional monomer further comprises a functional group A; and the functional group A is at least one of hydroxyl group, carboxyl group, amide group, sulfonic acid group, zwitterion, polyethylene glycol, urea group, carbamate group, carboxylate ion, sulfonate, sulfoxide, and pyrrolidone.

3. The preparation method of claim 1, wherein the first functional monomer is at least one of DL-2-amino-4-pentenoic acid, 2-methylallylamine, 3-butene-1-amine, 4-penten-1-amine, 2-aminoethyl methacrylate, methacryloyl hydrazide, acryloyl hydrazide, double bonded polylysine, 2-amino-7-en-octanoic acid, 6-en-aminoheptanoic acid, 2-aminopent-4-enoic acid, 4-(1-amino-2-methyl-propyl)-hepta-1,6-diene-4-ol, and 4-(1-amino-ethyl)-hepta-1,6-diene-4-ol.

4. The preparation method of claim 1, wherein step S100 comprises:
AS110: contacting the biological material with the first treatment solution for physical permeation, wherein the first treatment solution is a solution containing the first functional monomer; the active group is an amino group or a hydrazide; and
AS120: contacting the biological material treated in AS110 with the second treatment solution for co-crosslinking to introduce the first carbon-carbon double bonds, wherein the second treatment solution is an aldehyde-based cross-linking agent solution.

5. The preparation method of claim 4, wherein step S100 further comprises:
AS130: soaking the biological material treated in step AS120 in a solution containing a second functional monomer for physical permeation to introduce second carbon-carbon double bonds; wherein the second functional monomer has a second carbon-carbon double bond.

6. The preparation method of claim 5, wherein the second functional monomer further comprises a functional group B; and the functional group B is at least one of hydroxyl group, carboxyl group, choline carboxylate, choline sulfonate, choline phosphate, pyrrolidone, sulfonic acid group, carboxylate ion, sulfonate, sulfoxide, amide group and methoxy group.

7. The preparation method of claim 6, wherein the second functional monomer is at least one of polyethylene glycol diacrylate, 1,4-butanediol diacrylate, ethyl acrylate, N-methyl-2-acrylamide, N-2,2-propenyl-2-acrylamide, N-ethylacrylamide, N,N′-ethylenebisacrylamide, (ethane-1,2-diylbis(oxy))bis(ethane-2,1-diyl)diacrylate, double-bonded hyaluronic acid, acrylamide, 2-(prop-2-enoylamino)acetic acid, 2-acrylamido-2-methylpropanesulfonic acid, hydroxyethyl methacrylate, 3-[[2-(methacryloyloxy)ethyl]dimethylammonio]propionate, N-isopropylacrylamide, N-(hydroxymethyl)acrylamide, N-(2-hydroxyethyl)methacrylamide, N,N-dimethylmethacrylamide, 3-[N,N-dimethyl-[2-(2-methylprop-2-enoyloxy)ethyl]ammonium]propane-1-sulfonate, 2-methacryloyloxyethyl phosphorylcholine and double-bonded polylysine.

8. The preparation method of claim 4, wherein step S100 further comprises step AS120 (M) after step AS120:
soaking the biological material treated in step AS120 in a solution containing a third functional monomer to eliminate residual aldehyde groups; wherein the third functional monomer has an amino group or a hydrazide.

9. The preparation method of claim 8, wherein the third functional group further comprises a functional group C; and the functional group C is at least one of hydroxyl group, carboxyl group, amide group, sulfonic acid group, zwitterion, polyethylene glycol, urea group, carbamate group, carboxylate ion, sulfonate, sulfoxide, and pyrrolidone.

10. The preparation method of claim 9, wherein the third functional monomer is at least one of DL-2-amino-4-pentenoic acid, 2-methylallylamine, 3-butene-1-amine, 4-penten-1-amine, 2-aminoethyl methacrylate, methacryloyl hydrazide, acryloyl hydrazide, double bonded polylysine, 2-amino-7-en-octanoic acid, 6-en-aminoheptanoic acid, 2-aminopent-4-enoic acid, 4-(1-amino-2-methyl-propyl)-hepta-1,6-diene-4-ol, and 4-(1-amino-ethyl)-hepta-1,6-diene-4-ol.

11. The preparation method of claim 4, wherein,
in step S100:
the biological material is animal tissue, comprising one or more of pericardium, valve, intestinal membrane, meninges, pleura, blood vessel, skin or ligament; the animal tissue is a fresh animal tissue or a decellularized biological tissue;
the aldehyde-based cross-linking agent is glutaraldehyde or formaldehyde;
performing a non-condensation chemical reaction to graft the first carbon-carbon double bonds;
the biological material has not been subjected to any other chemical reaction involving any reagent before being treated with the aldehyde-based cross-linking agent;
in a reaction system of step S100, the first carbon-carbon double bond is provided by the first functional monomer with the active group, and reaction raw materials in step S100 consist of the biological material, the first functional monomer and the aldehyde-based cross-linking agent;
in step AS110, a solvent in the solution containing the first functional monomer is water, physiological saline, isopropanol, pH neutral buffer or ethanol aqueous solution; a concentration of the first functional monomer in the solution containing the first functional monomer is in a range of 10 to 100 mM; and a soaking time is in a range of 2 to 20 hours;
in step AS120, a final concentration of the aldehyde-based cross-linking agent in a reaction system of step AS120 is in a range of 10 to 800 mM; and a co-crosslinking time is in a range of 10 to 30 h;
in step S200:
the initiator is added to a system treated in a previous step; or the biological material treated in the previous step is taken out and directly soaked in a solution containing the initiator, or the biological material treated in the previous step is taken out and soaked in the solution containing the initiator after cleaning;
the initiator is a single initiator or a mixed initiator, and the mixed initiator is:
a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, wherein a concentration of each component in the mixture is in a range of 1 to 100 mM;
or the mixed initiator is:
a mixture of ammonium persulfate and N,N,N′,N′-tetramethylethylenediamine, or a mixture of potassium persulfate and N,N,N′,N′-tetramethylethylenediamine, or a mixture of ammonium persulfate and N,N,N′,N′-tetramethylethylenediamine, or a mixture of sodium persulfate and N,N,N′,N′-tetramethylethylenediamine, wherein mass percentage concentrations of ammonium persulfate, potassium persulfate or sodium persulfate are in a range of 2% to 5% respectively, and a mass percentage of tetramethylethylenediamine is in a range of 0.2% to 0.5%;
the single initiator is any component of the mixed initiator.

12. A preparation method for a biological valve material, comprising:
step S100: treating a biological material with a first treatment solution and a second treatment solution in sequence to obtain a pretreated biological material chemically grafted with first carbon-carbon double bonds; wherein the first treatment solution contains a reagent B, and the second treatment solution contains a reagent A; the reagent B is an aldehyde-based cross-linking agent; the reagent A is a first functional monomer with a first carbon-carbon double bond, the first functional monomer further comprises an active group for participating in a chemical reaction, and the active group is an active group for reacting with an aldehyde group;
step S200: performing polymerization of carbon-carbon double bonds under an action of an initiator to obtain the biological valve material.

13. The preparation method of claim 12, wherein step S100 comprises:
BS110: contacting the biological material with the first treatment solution for cross-linking, wherein the first treatment solution is an aldehyde-based cross-linking agent solution;
BS120: contacting the biological material treated in step BS110 with the second treatment solution for chemical reaction to introduce the first carbon-carbon double bonds, wherein the second treatment solution is a solution containing the first functional monomer; and the active group is an oxirane group.

14. The preparation method of claim 12, wherein the first functional monomer is at least one of allyl glycidyl ether, glycidyl methacrylate and glycidyl acrylate.

15. The preparation method of claim 12, wherein step S100 further comprises:
Step BS130: soaking the biological material treated in step BS120 in a solution containing a second functional monomer, wherein the second functional monomer has a second carbon-carbon double bond.

16. The preparation method of claim 15, wherein the second functional monomer is one or more of polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-ethylenebisacrylamide, (ethane-1,2-diylbis(oxy))bis(ethane-2,1-diyl)diacrylate, N,N′-dimethylacrylamide, N,N-dimethylmethacrylamide, and double bonded polylysine.

17. The preparation method of claim 15, wherein the second functional monomer further comprises a functional group B; the functional group B is at least one of hydroxyl group, carboxyl group, choline carboxylate, choline sulfonate, choline phosphate, pyrrolidone, sulfonic acid group, carboxylate ion, sulfonate, sulfoxide, amide group and methoxy group.

18. The preparation method of claim 17, wherein the second functional monomer is one or more of acrylamide, acrylic acid, sodium acrylate, methacrylic acid, sodium methacrylate, 2-(prop-2-enoylamino)acetic acid, 2-acrylamido-2-methylpropanesulfonic acid, hydroxyethyl methacrylate, 3-[[2-(methacryloyloxy)ethyl]dimethylammonio]propionate, N-methyl-2-acrylamide, N-isopropylacrylamide, N-(hydroxymethyl)acrylamide, N-(2-hydroxyethyl)methacrylamide, 3-[N,N-dimethyl-[2-(2-methylprop-2-enoyloxy)ethyl]ammonium]propane-1-sulfonate, 2-methacryloyloxyethyl phosphorylcholine, N-(2-hydroxyethyl)acrylamide, N-(methoxymethyl)methacrylamide, 2-acrylamido-2-methylpropanesulfonic acid, and double-bonded hyaluronic acid.

19. The preparation method of claim 15, wherein,
in step BS110:
a w/w concentration of the aldehyde-based cross-linking agent solution is in a range of 0.1% to 5%; and a crosslinking time is in a range of 0.5 h to 120 h;
in step BS120:
a solution containing the first functional monomer consists of the first functional monomer and a solvent that does not participate in chemical reaction;
a w/w concentration of the first functional monomer in the solution containing the first functional monomer is in a range of 1% to 10%; and a reaction time is in a range of 2 to 120 hours;
the solvent in the solution containing the first functional monomer is one or more of an aqueous solution of any one of methanol, ethanol, ethylene glycol, propanol, 1,2-propanediol, 1,3-propanediol, isopropanol, butanol, isobutanol, 1,2-butanediol, 1,3-butanediol, 1,4-butanediol, and glycerol, water, physiological saline, and a neutral pH buffer;
in step BS130:
the second functional monomer enters the biological material by physical permeation; the solution containing the second functional monomer consists of the second functional monomer and a solvent that does not participate in reaction;
a v/v concentration of the second functional monomer in the solution containing the second functional monomer is in a range of 0.1% to 20%; and a soaking time is in a range of 0.5 h to 120 h.

20. A heart valve, comprising a stent and a leaflet, wherein the leaflet is prepared by:
step S100: providing a biological material, and treating the biological material with a first treatment solution and a second treatment solution in sequence to obtain a pre-treated biological material chemically grafted with first carbon-carbon double bonds; wherein the first treatment solution and the second treatment solution respectively contain one of a reagent A and a reagent B, wherein the reagent A is a first functional monomer with a first carbon-carbon double bond, and the reagent B is an aldehyde-based cross-linking agent; and
step S200: performing polymerization of carbon-carbon double bonds under an action of an initiator to obtain the leaflet.
